SEQUENCE LISTING (652 sequences)

SEQ ID NO: 1 - DNA, 20 bp, synthetic construct
  source 1..20: mol_type = other DNA
atggcaacccttggacagta

SEQ ID NO: 2 - DNA, 22 bp, synthetic construct
  source 1..22: mol_type = other DNA
tccataccagtggaaggagaga

SEQ ID NO: 3 - DNA, 24 bp, synthetic construct
  source 1..24: mol_type = other DNA
cgtcatagagcccttcttttacct

SEQ ID NO: 4 - DNA, 24 bp, synthetic construct
  source 1..24: mol_type = other DNA
tgatgctgaatgaattcttgaacc

SEQ ID NO: 5 - DNA, 20 bp, synthetic construct
  source 1..20: mol_type = other DNA
ttatcctgagcggtgctgtt

SEQ ID NO: 6 - DNA, 25 bp, synthetic construct
  source 1..25: mol_type = other DNA
ggatccatacaacagcatattccaa

SEQ ID NO: 7 - DNA, 25 bp, synthetic construct
  source 1..25: mol_type = other DNA
gcttccaagaaaaacaagtttgtca

SEQ ID NO: 8 - DNA, 21 bp, synthetic construct
  source 1..21: mol_type = other DNA
agctccgttgaagagaagcaa

SEQ ID NO: 9 - DNA, 22 bp, synthetic construct
  source 1..22: mol_type = other DNA
actctctcaaagacccttcagc

SEQ ID NO: 10 - DNA, 25 bp, synthetic construct
  source 1..25: mol_type = other DNA
acaaatatgccagttccaccaattt

SEQ ID NO: 11 - DNA, 23 bp, synthetic construct
  source 1..23: mol_type = other DNA
acaaatacaccaagcaagatggg

SEQ ID NO: 12 - DNA, 23 bp, synthetic construct
  source 1..23: mol_type = other DNA
ccagcatatgcatgatgacatcc

SEQ ID NO: 13 - DNA, 25 bp, synthetic construct
  source 1..25: mol_type = other DNA
atgataactgagcagttgaagaaga

SEQ ID NO: 14 - DNA, 20 bp, synthetic construct
  source 1..20: mol_type = other DNA
cgtccatcaaagcctggtac

SEQ ID NO: 15 - DNA, 26 bp, synthetic construct
  source 1..26: mol_type = other DNA
tgaagttgaagctgttgatagtgaat

SEQ ID NO: 16 - DNA, 21 bp, synthetic construct
  source 1..21: mol_type = other DNA
cttgagcgtctcagcatttcc

SEQ ID NO: 17 - DNA, 27 bp, synthetic construct
  source 1..27: mol_type = other DNA
cggtgtttcaaaaatcaaaaatttggt

SEQ ID NO: 18 - DNA, 26 bp, synthetic construct
  source 1..26: mol_type = other DNA
tggttgtttggattcaatcgtttaca

SEQ ID NO: 19 - DNA, 19 bp, synthetic construct
  source 1..19: mol_type = other DNA
acctgcccgaaaaccactg

SEQ ID NO: 20 - DNA, 20 bp, synthetic construct
  source 1..20: mol_type = other DNA
atgtcccttctgcctccttc

SEQ ID NO: 21 - DNA, 22 bp, synthetic construct
  source 1..22: mol_type = other DNA
ccaactgcagcatctatgaacc

SEQ ID NO: 22 - DNA, 18 bp, synthetic construct
  source 1..18: mol_type = other DNA
acctcgtctggtccccaa

SEQ ID NO: 23 - DNA, 18 bp, synthetic construct
  source 1..18: mol_type = other DNA
gtctgtggcagctgcacc

SEQ ID NO: 24 - DNA, 18 bp, synthetic construct
  source 1..18: mol_type = other DNA
ccacatgagcggcaggtg

SEQ ID NO: 25 - DNA, 20 bp, synthetic construct
  source 1..20: mol_type = other DNA
gtggaagttgtggtggctgt

SEQ ID NO: 26 - DNA, 18 bp, synthetic construct
  source 1..18: mol_type = other DNA
gtagcacctgcaggtggt

SEQ ID NO: 27 - DNA, 18 bp, synthetic construct
  source 1..18: mol_type = other DNA
agcacgcccgtgatctgt

SEQ ID NO: 28 - DNA, 21 bp, synthetic construct
  source 1..21: mol_type = other DNA
ctagcagcagcattgcttctt

SEQ ID NO: 29 - DNA, 21 bp, synthetic construct
  source 1..21: mol_type = other DNA
cacgcagagggatttggaatt

SEQ ID NO: 30 - DNA, 23 bp, synthetic construct
  source 1..23: mol_type = other DNA
tggcagtcaaactcttcattacc

SEQ ID NO: 31 - DNA, 21 bp, synthetic construct
  source 1..21: mol_type = other DNA
gctgctgcctctacgttcaat

SEQ ID NO: 32 - DNA, 20 bp, synthetic construct
  source 1..20: mol_type = other DNA
gctggcatcctgttcctcta

SEQ ID NO: 33 - DNA, 27 bp, synthetic construct
  source 1..27: mol_type = other DNA
gctaccatagatgtgacatttttgacc

SEQ ID NO: 34 - DNA, 26 bp, synthetic construct
  source 1..26: mol_type = other DNA
tgcactgtgctgaaaatgaaatatct

SEQ ID NO: 35 - DNA, 22 bp, synthetic construct
  source 1..22: mol_type = other DNA
tgatgctctattgtgcagtagc

SEQ ID NO: 36 - DNA, 20 bp, synthetic construct
  source 1..20: mol_type = other DNA
agcatgccaaaaggtgttcc

SEQ ID NO: 37 - DNA, 22 bp, synthetic construct
  source 1..22: mol_type = other DNA
ctcagccagcctattcttttgg

SEQ ID NO: 38 - DNA, 26 bp, synthetic construct
  source 1..26: mol_type = other DNA
tcctaagaaaagatagttggcttctg

SEQ ID NO: 39 - DNA, 24 bp, synthetic construct
  source 1..24: mol_type = other DNA
aaattgggacaacatactttacgc

SEQ ID NO: 40 - DNA, 31 bp, synthetic construct
  source 1..31: mol_type = other DNA
ccaatcacaattgtatctaagtttcactttt

SEQ ID NO: 41 - DNA, 21 bp, synthetic construct
  source 1..21: mol_type = other DNA
acatttggctccccagtcatc

SEQ ID NO: 42 - DNA, 25 bp, synthetic construct
  source 1..25: mol_type = other DNA
tcctcttctttaagaccagctttca

SEQ ID NO: 43 - DNA, 26 bp, synthetic construct
  source 1..26: mol_type = other DNA
tctgatgatgatcggactaaagaaga

SEQ ID NO: 44 - DNA, 20 bp, synthetic construct
  source 1..20: mol_type = other DNA
acatgcccaagtacgcagat

SEQ ID NO: 45 - DNA, 21 bp, synthetic construct
  source 1..21: mol_type = other DNA
ctaccttgtatgccagcacct

SEQ ID NO: 46 - DNA, 28 bp, synthetic construct
  source 1..28: mol_type = other DNA
aggacaaaacagatcaagattaaaacag

SEQ ID NO: 47 - DNA, 19 bp, synthetic construct
  source 1..19: mol_type = other DNA
ggcatgtggacacagggaa

SEQ ID NO: 48 - DNA, 28 bp, synthetic construct
  source 1..28: mol_type = other DNA
gtctgttcattaatagcttcaagaggaa

SEQ ID NO: 49 - DNA, 22 bp, synthetic construct
  source 1..22: mol_type = other DNA
acaagccaggtcctaacataga

SEQ ID NO: 50 - DNA, 23 bp, synthetic construct
  source 1..23: mol_type = other DNA
cgcagtttcgctttttcttcatc

SEQ ID NO: 51 - DNA, 24 bp, synthetic construct
  source 1..24: mol_type = other DNA
tcctgatatttgggcttagtgctt

SEQ ID NO: 52 - DNA, 25 bp, synthetic construct
  source 1..25: mol_type = other DNA
tggaactttttcctcaaacaaacac

SEQ ID NO: 53 - DNA, 22 bp, synthetic construct
  source 1..22: mol_type = other DNA
aggatgctggattggatgattg

SEQ ID NO: 54 - DNA, 23 bp, synthetic construct
  source 1..23: mol_type = other DNA
ttcttcttcctcttcttcctcct

SEQ ID NO: 55 - DNA, 22 bp, synthetic construct
  source 1..22: mol_type = other DNA
tcaaagtttctcaatggccagt

SEQ ID NO: 56 - DNA, 27 bp, synthetic construct
  source 1..27: mol_type = other DNA
catgcggagaaatctacatattttgaa

SEQ ID NO: 57 - DNA, 20 bp, synthetic construct
  source 1..20: mol_type = other DNA
ctgagggactggctgagaac

SEQ ID NO: 58 - DNA, 22 bp, synthetic construct
  source 1..22: mol_type = other DNA
tcatcatccctcaccagagttg

SEQ ID NO: 59 - DNA, 21 bp, synthetic construct
  source 1..21: mol_type = other DNA
gcgagatccgtgttctccatt

SEQ ID NO: 60 - DNA, 23 bp, synthetic construct
  source 1..23: mol_type = other DNA
acatgaggcaaagattaagcact

SEQ ID NO: 61 - DNA, 26 bp, synthetic construct
  source 1..26: mol_type = other DNA
tggcattttatctggaatagcatctg

SEQ ID NO: 62 - DNA, 22 bp, synthetic construct
  source 1..22: mol_type = other DNA
tgcatatgtgcaagtctgcatg

SEQ ID NO: 63 - DNA, 22 bp, synthetic construct
  source 1..22: mol_type = other DNA
caaaagccagagctcagcttta

SEQ ID NO: 64 - DNA, 20 bp, synthetic construct
  source 1..20: mol_type = other DNA
cacagaaagctgcagagtcc

SEQ ID NO: 65 - DNA, 23 bp, synthetic construct
  source 1..23: mol_type = other DNA
tgattctgacattagcagtgtgg

SEQ ID NO: 66 - DNA, 21 bp, synthetic construct
  source 1..21: mol_type = other DNA
gaccagattcctgacagagct

SEQ ID NO: 67 - DNA, 19 bp, synthetic construct
  source 1..19: mol_type = other DNA
gaacggctgcggacacctg

SEQ ID NO: 68 - DNA, 18 bp, synthetic construct
  source 1..18: mol_type = other DNA
gctcgcacagctgcccgt

SEQ ID NO: 69 - DNA, 22 bp, synthetic construct
  source 1..22: mol_type = other DNA
ccagaccttaaccaaagatgct

SEQ ID NO: 70 - DNA, 23 bp, synthetic construct
  source 1..23: mol_type = other DNA
agagagacaaaaagcatcctcct

SEQ ID NO: 71 - DNA, 24 bp, synthetic construct
  source 1..24: mol_type = other DNA
aggcaacgtttataacagtcacat

SEQ ID NO: 72 - DNA, 21 bp, synthetic construct
  source 1..21: mol_type = other DNA
ggtttagcatagtcccatggc

SEQ ID NO: 73 - DNA, 21 bp, synthetic construct
  source 1..21: mol_type = other DNA
ctatgtggatggctactccgt

SEQ ID NO: 74 - DNA, 22 bp, synthetic construct
  source 1..22: mol_type = other DNA
ggattccaacagtgaagcagaa

SEQ ID NO: 75 - DNA, 22 bp, synthetic construct
  source 1..22: mol_type = other DNA
acattagccctctggttttcca

SEQ ID NO: 76 - DNA, 27 bp, synthetic construct
  source 1..27: mol_type = other DNA
ctagtgatgaaactaaccaaactatgc

SEQ ID NO: 77 - DNA, 23 bp, synthetic construct
  source 1..23: mol_type = other DNA
cccattctttcaacccccttaac

SEQ ID NO: 78 - DNA, 32 bp, synthetic construct
  source 1..32: mol_type = other DNA
cccccatataaatatgaagagaaaaatagaca

SEQ ID NO: 79 - DNA, 20 bp, synthetic construct
  source 1..20: mol_type = other DNA
aatcctccctgcactgcatt

SEQ ID NO: 80 - DNA, 25 bp, synthetic construct
  source 1..25: mol_type = other DNA
cctgggctattgataaagcatgaaa

SEQ ID NO: 81 - DNA, 21 bp, synthetic construct
  source 1..21: mol_type = other DNA
ggagaatgctgcaatcttggg

SEQ ID NO: 82 - DNA, 26 bp, synthetic construct
  source 1..26: mol_type = other DNA
agtggtgtggatcattttcttgttac

SEQ ID NO: 83 - DNA, 20 bp, synthetic construct
  source 1..20: mol_type = other DNA
tatctcagcctcgccaacaa

SEQ ID NO: 84 - DNA, 19 bp, synthetic construct
  source 1..19: mol_type = other DNA
ggttgctgcactgggagaa

SEQ ID NO: 85 - DNA, 19 bp, synthetic construct
  source 1..19: mol_type = other DNA
gctgccctagcgagtgtac

SEQ ID NO: 86 - DNA, 22 bp, synthetic construct
  source 1..22: mol_type = other DNA
cattgagttcagtgatgtgcgt

SEQ ID NO: 87 - DNA, 24 bp, synthetic construct
  source 1..24: mol_type = other DNA
gcctttcatttttggtagctgctt

SEQ ID NO: 88 - DNA, 22 bp, synthetic construct
  source 1..22: mol_type = other DNA
gttccagttctctcctgtcact

SEQ ID NO: 89 - DNA, 22 bp, synthetic construct
  source 1..22: mol_type = other DNA
tgggactataggcacacatcac

SEQ ID NO: 90 - DNA, 20 bp, synthetic construct
  source 1..20: mol_type = other DNA
cgaggcgagtgaattgcttg

SEQ ID NO: 91 - DNA, 26 bp, synthetic construct
  source 1..26: mol_type = other DNA
ccagctttaaattatctcttcatgcc

SEQ ID NO: 92 - DNA, 21 bp, synthetic construct
  source 1..21: mol_type = other DNA
tgatccaccatgtcagtctcc

SEQ ID NO: 93 - DNA, 28 bp, synthetic construct
  source 1..28: mol_type = other DNA
ggaaagccttaggtattcacacatttaa

SEQ ID NO: 94 - DNA, 28 bp, synthetic construct
  source 1..28: mol_type = other DNA
actcaaatcctactaacaaatgttacca

SEQ ID NO: 95 - DNA, 21 bp, synthetic construct
  source 1..21: mol_type = other DNA
tcttgtcctcctatccacctg

SEQ ID NO: 96 - DNA, 20 bp, synthetic construct
  source 1..20: mol_type = other DNA
agtggctatagtgcccccaa

SEQ ID NO: 97 - DNA, 20 bp, synthetic construct
  source 1..20: mol_type = other DNA
cctcggcggaaaagaaagtg

SEQ ID NO: 98 - DNA, 19 bp, synthetic construct
  source 1..19: mol_type = other DNA
ctgtttgctccggtccgac

SEQ ID NO: 99 - DNA, 24 bp, synthetic construct
  source 1..24: mol_type = other DNA
agagtcagctagcagattagaaac

SEQ ID NO: 100 - DNA, 22 bp, synthetic construct
  source 1..22: mol_type = other DNA
actcatcctcgtttccactcag

SEQ ID NO: 101 - DNA, 20 bp, synthetic construct
  source 1..20: mol_type = other DNA
ccctaacggagcccccagtg

SEQ ID NO: 102 - DNA, 18 bp, synthetic construct
  source 1..18: mol_type = other DNA
cgcggagctggccgtgtg

SEQ ID NO: 103 - DNA, 20 bp, synthetic construct
  source 1..20: mol_type = other DNA
cctgggccttctctttcact

SEQ ID NO: 104 - DNA, 24 bp, synthetic construct
  source 1..24: mol_type = other DNA
cacaggcaaaacacatcctttatg

SEQ ID NO: 105 - DNA, 22 bp, synthetic construct
  source 1..22: mol_type = other DNA
tctctggagtctatggtaggca

SEQ ID NO: 106 - DNA, 23 bp, synthetic construct
  source 1..23: mol_type = other DNA
cacaaaaggcctaaaatcagagc

SEQ ID NO: 107 - DNA, 21 bp, synthetic construct
  source 1..21: mol_type = other DNA
ggcactctattcactgcagga

SEQ ID NO: 108 - DNA, 21 bp, synthetic construct
  source 1..21: mol_type = other DNA
tgctcctctcactctctctct

SEQ ID NO: 109 - DNA, 24 bp, synthetic construct
  source 1..24: mol_type = other DNA
caagcaatcttggaatcctcaact

SEQ ID NO: 110 - DNA, 26 bp, synthetic construct
  source 1..26: mol_type = other DNA
aaatgtttagcacttttgttcaaagc

SEQ ID NO: 111 - DNA, 24 bp, synthetic construct
  source 1..24: mol_type = other DNA
ggtatttacacctggaaaatcaga

SEQ ID NO: 112 - DNA, 26 bp, synthetic construct
  source 1..26: mol_type = other DNA
gcttggtttattcctgttagacactc

SEQ ID NO: 113 - DNA, 27 bp, synthetic construct
  source 1..27: mol_type = other DNA
agacaaaggcttaagtttgaaaggtag

SEQ ID NO: 114 - DNA, 21 bp, synthetic construct
  source 1..21: mol_type = other DNA
ggctttcccattgtgctatgc

SEQ ID NO: 115 - DNA, 23 bp, synthetic construct
  source 1..23: mol_type = other DNA
aaatcctgacttcacaggagaac

SEQ ID NO: 116 - DNA, 20 bp, synthetic construct
  source 1..20: mol_type = other DNA
tctccttgggccagtgaaag

SEQ ID NO: 117 - DNA, 23 bp, synthetic construct
  source 1..23: mol_type = other DNA
agctagtaaaacagaaggtgcca

SEQ ID NO: 118 - DNA, 23 bp, synthetic construct
  source 1..23: mol_type = other DNA
cacaccattttggccagtaattg

SEQ ID NO: 119 - DNA, 20 bp, synthetic construct
  source 1..20: mol_type = other DNA
gcaacactgccaccaaaatc

SEQ ID NO: 120 - DNA, 20 bp, synthetic construct
  source 1..20: mol_type = other DNA
aattcagctttgccctccca

SEQ ID NO: 121 - DNA, 19 bp, synthetic construct
  source 1..19: mol_type = other DNA
gagtcgcgcaggctggaag

SEQ ID NO: 122 - DNA, 20 bp, synthetic construct
  source 1..20: mol_type = other DNA
cttcacgaacgcgagcttct

SEQ ID NO: 123 - DNA, 23 bp, synthetic construct
  source 1..23: mol_type = other DNA
gggttttccctgttttcagttgt

SEQ ID NO: 124 - DNA, 28 bp, synthetic construct
  source 1..28: mol_type = other DNA
gtcaaacattttatttggtgtttcatgc

SEQ ID NO: 125 - DNA, 26 bp, synthetic construct
  source 1..26: mol_type = other DNA
gccatgaataaggaactcatcgaaaa

SEQ ID NO: 126 - DNA, 26 bp, synthetic construct
  source 1..26: mol_type = other DNA
cattgaaatagcccctcttatatgct

SEQ ID NO: 127 - DNA, 20 bp, synthetic construct
  source 1..20: mol_type = other DNA
ttcgcaggcagatagagcac

SEQ ID NO: 128 - DNA, 25 bp, synthetic construct
  source 1..25: mol_type = other DNA
tcagcaattgcttgtgaatcattct

SEQ ID NO: 129 - DNA, 21 bp, synthetic construct
  source 1..21: mol_type = other DNA
tcacagagaacctgaccaagg

SEQ ID NO: 130 - DNA, 22 bp, synthetic construct
  source 1..22: mol_type = other DNA
tggctccttagttccaagatgg

SEQ ID NO: 131 - DNA, 24 bp, synthetic construct
  source 1..24: mol_type = other DNA
tgatcgacaaagaaacaaatgacc

SEQ ID NO: 132 - DNA, 21 bp, synthetic construct
  source 1..21: mol_type = other DNA
tcttgctcctgaaccttcagt

SEQ ID NO: 133 - DNA, 22 bp, synthetic construct
  source 1..22: mol_type = other DNA
agtaaaggctgttctggctttt

SEQ ID NO: 134 - DNA, 28 bp, synthetic construct
  source 1..28: mol_type = other DNA
agaagagaagaaacaaatcctaagttcg

SEQ ID NO: 135 - DNA, 25 bp, synthetic construct
  source 1..25: mol_type = other DNA
aagaacttgcacagtgaaatatctg

SEQ ID NO: 136 - DNA, 21 bp, synthetic construct
  source 1..21: mol_type = other DNA
agcagagtttccagtcctgag

SEQ ID NO: 137 - DNA, 20 bp, synthetic construct
  source 1..20: mol_type = other DNA
gcttacccagcccttacaac

SEQ ID NO: 138 - DNA, 20 bp, synthetic construct
  source 1..20: mol_type = other DNA
aagaattccctgtggcgtca

SEQ ID NO: 139 - DNA, 20 bp, synthetic construct
  source 1..20: mol_type = other DNA
ttctgttcccaggcatctcc

SEQ ID NO: 140 - DNA, 21 bp, synthetic construct
  source 1..21: mol_type = other DNA
gagaacaccaccttacgggtt

SEQ ID NO: 141 - DNA, 20 bp, synthetic construct
  source 1..20: mol_type = other DNA
tactctcctacctcgcctcc

SEQ ID NO: 142 - DNA, 22 bp, synthetic construct
  source 1..22: mol_type = other DNA
tgtctactgatagttcctgccg

SEQ ID NO: 143 - DNA, 23 bp, synthetic construct
  source 1..23: mol_type = other DNA
agccacagaatctaactggtgag

SEQ ID NO: 144 - DNA, 22 bp, synthetic construct
  source 1..22: mol_type = other DNA
tcccttccctcttaaggtcaaa

SEQ ID NO: 145 - DNA, 25 bp, synthetic construct
  source 1..25: mol_type = other DNA
atgctaaagtaaggattctgtggat

SEQ ID NO: 146 - DNA, 22 bp, synthetic construct
  source 1..22: mol_type = other DNA
tcccaaatcagccagattttcc

SEQ ID NO: 147 - DNA, 23 bp, synthetic construct
  source 1..23: mol_type = other DNA
cagcgtttcaccatgttagctag

SEQ ID NO: 148 - DNA, 28 bp, synthetic construct
  source 1..28: mol_type = other DNA
aatacttcatatagaactgaaacttggc

SEQ ID NO: 149 - DNA, 20 bp, synthetic construct
  source 1..20: mol_type = other DNA
tctggctccgagaagaacac

SEQ ID NO: 150 - DNA, 19 bp, synthetic construct
  source 1..19: mol_type = other DNA
gggagttctgccggtccat

SEQ ID NO: 151 - DNA, 26 bp, synthetic construct
  source 1..26: mol_type = other DNA
tctttcagtttggatgatttggtttg

SEQ ID NO: 152 - DNA, 23 bp, synthetic construct
  source 1..23: mol_type = other DNA
gttgcatcaaacactgtcagttc

SEQ ID NO: 153 - DNA, 27 bp, synthetic construct
  source 1..27: mol_type = other DNA
gggtgaattatcccattctagttacag

SEQ ID NO: 154 - DNA, 27 bp, synthetic construct
  source 1..27: mol_type = other DNA
ttttagatgagttaattttcccccaac

SEQ ID NO: 155 - DNA, 19 bp, synthetic construct
  source 1..19: mol_type = other DNA
ccagggagtctgtcacctg

SEQ ID NO: 156 - DNA, 20 bp, synthetic construct
  source 1..20: mol_type = other DNA
agcacaggctttctcccttc

SEQ ID NO: 157 - DNA, 20 bp, synthetic construct
  source 1..20: mol_type = other DNA
ctcaggtcaggaggagtgag

SEQ ID NO: 158 - DNA, 21 bp, synthetic construct
  source 1..21: mol_type = other DNA
ctgtgagaagcaacactgagg

SEQ ID NO: 159 - DNA, 21 bp, synthetic construct
  source 1..21: mol_type = other DNA
tctgactcagagcgataagcc

SEQ ID NO: 160 - DNA, 22 bp, synthetic construct
  source 1..22: mol_type = other DNA
aggacgagactgaaagtcaagg

SEQ ID NO: 161 - DNA, 24 bp, synthetic construct
  source 1..24: mol_type = other DNA
cagagtcctctaccattcactcag

SEQ ID NO: 162 - DNA, 20 bp, synthetic construct
  source 1..20: mol_type = other DNA
aggctggctaaagtgcagaa

SEQ ID NO: 163 - DNA, 20 bp, synthetic construct
  source 1..20: mol_type = other DNA
cctttgtgggtgacttcagg

SEQ ID NO: 164 - DNA, 21 bp, synthetic construct
  source 1..21: mol_type = other DNA
ggaagccaacctgggtctaaa

SEQ ID NO: 165 - DNA, 21 bp, synthetic construct
  source 1..21: mol_type = other DNA
caggctggagttggtgttaga

SEQ ID NO: 166 - DNA, 22 bp, synthetic construct
  source 1..22: mol_type = other DNA
tcgatcatagagtcagaccaca

SEQ ID NO: 167 - DNA, 21 bp, synthetic construct
  source 1..21: mol_type = other DNA
cctagttcccaccacacatga

SEQ ID NO: 168 - DNA, 20 bp, synthetic construct
  source 1..20: mol_type = other DNA
tggccaccaaattcccaaag

SEQ ID NO: 169 - DNA, 23 bp, synthetic construct
  source 1..23: mol_type = other DNA
ctggaaggtctgttttggtgtta

SEQ ID NO: 170 - DNA, 21 bp, synthetic construct
  source 1..21: mol_type = other DNA
gcctgcatgactttgccaatc

SEQ ID NO: 171 - DNA, 22 bp, synthetic construct
  source 1..22: mol_type = other DNA
gccatgttgcggtagtttgttg

SEQ ID NO: 172 - DNA, 18 bp, synthetic construct
  source 1..18: mol_type = other DNA
ccgggtacgggtgacgga

SEQ ID NO: 173 - DNA, 19 bp, synthetic construct
  source 1..19: mol_type = other DNA
gggctctcagcctcctaga

SEQ ID NO: 174 - DNA, 20 bp, synthetic construct
  source 1..20: mol_type = other DNA
agacctgcttcagctcttca

SEQ ID NO: 175 - DNA, 20 bp, synthetic construct
  source 1..20: mol_type = other DNA
tgcttcctacacagcctcct

SEQ ID NO: 176 - DNA, 18 bp, synthetic construct
  source 1..18: mol_type = other DNA
ggtgctgcaccggaagca

SEQ ID NO: 177 - DNA, 20 bp, synthetic construct
  source 1..20: mol_type = other DNA
ggcccttcattcctcctaga

SEQ ID NO: 178 - DNA, 20 bp, synthetic construct
  source 1..20: mol_type = other DNA
ctgaggttgctgaggctgag

SEQ ID NO: 179 - DNA, 22 bp, synthetic construct
  source 1..22: mol_type = other DNA
tgtgctcatagaccatcagacg

SEQ ID NO: 180 - DNA, 21 bp, synthetic construct
  source 1..21: mol_type = other DNA
ccgcagggactagagagtttc

SEQ ID NO: 181 - DNA, 20 bp, synthetic construct
  source 1..20: mol_type = other DNA
aagtgtacgagctgttccca

SEQ ID NO: 182 - DNA, 24 bp, synthetic construct
  source 1..24: mol_type = other DNA
ggtcttagttgagcaatttggcta

SEQ ID NO: 183 - DNA, 23 bp, synthetic construct
  source 1..23: mol_type = other DNA
cagagaatgtttgggaagactgg

SEQ ID NO: 184 - DNA, 26 bp, synthetic construct
  source 1..26: mol_type = other DNA
gggaaatgaggttaacaaagattcgt

SEQ ID NO: 185 - DNA, 22 bp, synthetic construct
  source 1..22: mol_type = other DNA
actttctgaagatcgagctcgt

SEQ ID NO: 186 - DNA, 20 bp, synthetic construct
  source 1..20: mol_type = other DNA
ttcaggtgcacaagctccat

SEQ ID NO: 187 - DNA, 20 bp, synthetic construct
  source 1..20: mol_type = other DNA
tctacctgccctggatctca

SEQ ID NO: 188 - DNA, 25 bp, synthetic construct
  source 1..25: mol_type = other DNA
gtccccaaaggaactttaaatctgt

SEQ ID NO: 189 - DNA, 21 bp, synthetic construct
  source 1..21: mol_type = other DNA
cgtttgaccaggccaataacc

SEQ ID NO: 190 - DNA, 20 bp, synthetic construct
  source 1..20: mol_type = other DNA
tcccccttatagggtgtgga

SEQ ID NO: 191 - DNA, 20 bp, synthetic construct
  source 1..20: mol_type = other DNA
gaaggtgctcacctgctact

SEQ ID NO: 192 - DNA, 20 bp, synthetic construct
  source 1..20: mol_type = other DNA
aggaactggatgacgctgac

SEQ ID NO: 193 - DNA, 22 bp, synthetic construct
  source 1..22: mol_type = other DNA
ttcatgcatctacactccatcg

SEQ ID NO: 194 - DNA, 22 bp, synthetic construct
  source 1..22: mol_type = other DNA
cgtacgctcaggagtaataggg

SEQ ID NO: 195 - DNA, 20 bp, synthetic construct
  source 1..20: mol_type = other DNA
gcgtctcttctctgcactcc

SEQ ID NO: 196 - DNA, 20 bp, synthetic construct
  source 1..20: mol_type = other DNA
gtgctcggtcctaaggtcag

SEQ ID NO: 197 - DNA, 19 bp, synthetic construct
  source 1..19: mol_type = other DNA
ctgactctgcggccctcca

SEQ ID NO: 198 - DNA, 20 bp, synthetic construct
  source 1..20: mol_type = other DNA
cttccagtccaggagcctgc

SEQ ID NO: 199 - DNA, 18 bp, synthetic construct
  source 1..18: mol_type = other DNA
gagatccgcgaggccttc

SEQ ID NO: 200 - DNA, 20 bp, synthetic construct
  source 1..20: mol_type = other DNA
gatcatctcgtccacctcct

SEQ ID NO: 201 - DNA, 19 bp, synthetic construct
  source 1..19: mol_type = other DNA
tggactggaagctccctcc

SEQ ID NO: 202 - DNA, 20 bp, synthetic construct
  source 1..20: mol_type = other DNA
ggaaggcagtgctgttttca

SEQ ID NO: 203 - DNA, 25 bp, synthetic construct
  source 1..25: mol_type = other DNA
gcattagtagcacacagagatcaga

SEQ ID NO: 204 - DNA, 22 bp, synthetic construct
  source 1..22: mol_type = other DNA
tgtcgaaggcctttactttgga

SEQ ID NO: 205 - DNA, 20 bp, synthetic construct
  source 1..20: mol_type = other DNA
gcgtcatccccatcaactcc

SEQ ID NO: 206 - DNA, 24 bp, synthetic construct
  source 1..24: mol_type = other DNA
tctctagaaaagtccatggaagcc

SEQ ID NO: 207 - DNA, 20 bp, synthetic construct
  source 1..20: mol_type = other DNA
cgcaaacagagtccctcaag

SEQ ID NO: 208 - DNA, 21 bp, synthetic construct
  source 1..21: mol_type = other DNA
gtgaaggcccaggtccttaaa

SEQ ID NO: 209 - DNA, 18 bp, synthetic construct
  source 1..18: mol_type = other DNA
gctgggcgtggtcacgaa

SEQ ID NO: 210 - DNA, 20 bp, synthetic construct
  source 1..20: mol_type = other DNA
acagcttgccgtaagtgagg

SEQ ID NO: 211 - DNA, 26 bp, synthetic construct
  source 1..26: mol_type = other DNA
ggaaagattgggaatcatggtattct

SEQ ID NO: 212 - DNA, 20 bp, synthetic construct
  source 1..20: mol_type = other DNA
aaatgtgcgtctgctccaag

SEQ ID NO: 213 - DNA, 20 bp, synthetic construct
  source 1..20: mol_type = other DNA
tccagcatgggcacttagaa

SEQ ID NO: 214 - DNA, 25 bp, synthetic construct
  source 1..25: mol_type = other DNA
cgtttccagatctgagacttagatg

SEQ ID NO: 215 - DNA, 26 bp, synthetic construct
  source 1..26: mol_type = other DNA
tgtcctgataagcactttctagacta

SEQ ID NO: 216 - DNA, 22 bp, synthetic construct
  source 1..22: mol_type = other DNA
acagcctttttcatcacaggga

SEQ ID NO: 217 - DNA, 24 bp, synthetic construct
  source 1..24: mol_type = other DNA
aaacgttttgtaccgattatccac

SEQ ID NO: 218 - DNA, 26 bp, synthetic construct
  source 1..26: mol_type = other DNA
cattcttagcttttcttgcagtttgt

SEQ ID NO: 219 - DNA, 23 bp, synthetic construct
  source 1..23: mol_type = other DNA
gcatgttggttaattgtggccat

SEQ ID NO: 220 - DNA, 26 bp, synthetic construct
  source 1..26: mol_type = other DNA
agataggtctgaaatttttacccagt

SEQ ID NO: 221 - DNA, 23 bp, synthetic construct
  source 1..23: mol_type = other DNA
accctgaccacatagtgtgatag

SEQ ID NO: 222 - DNA, 21 bp, synthetic construct
  source 1..21: mol_type = other DNA
ttttggcctttaacagccagt

SEQ ID NO: 223 - DNA, 21 bp, synthetic construct
  source 1..21: mol_type = other DNA
tccaggtcattttgctttggg

SEQ ID NO: 224 - DNA, 24 bp, synthetic construct
  source 1..24: mol_type = other DNA
acacaagtccatcattatcacgtc

SEQ ID NO: 225 - DNA, 20 bp, synthetic construct
  source 1..20: mol_type = other DNA
ctgagcaacgacgtcaagaa

SEQ ID NO: 226 - DNA, 18 bp, synthetic construct
  source 1..18: mol_type = other DNA
ctccaagctgcgctgagt

SEQ ID NO: 227 - DNA, 21 bp, synthetic construct
  source 1..21: mol_type = other DNA
gcttccagtactagcctctct

SEQ ID NO: 228 - DNA, 21 bp, synthetic construct
  source 1..21: mol_type = other DNA
agtaaggtgaccagcttggac

SEQ ID NO: 229 - DNA, 20 bp, synthetic construct
  source 1..20: mol_type = other DNA
acatcctcaagggcagctag

SEQ ID NO: 230 - DNA, 20 bp, synthetic construct
  source 1..20: mol_type = other DNA
gtgagccaagatcgcaccat

SEQ ID NO: 231 - DNA, 20 bp, synthetic construct
  source 1..20: mol_type = other DNA
ggacgtgagcctgagtttgc

SEQ ID NO: 232 - DNA, 18 bp, synthetic construct
  source 1..18: mol_type = other DNA
catcacgcgggacgtcac

SEQ ID NO: 233 - DNA, 19 bp, synthetic construct
  source 1..19: mol_type = other DNA
ccttccgatgggtctgctg

SEQ ID NO: 234 - DNA, 23 bp, synthetic construct
  source 1..23: mol_type = other DNA
cgaggaacaagggctgtttatct

SEQ ID NO: 235 - DNA, 20 bp, synthetic construct
  source 1..20: mol_type = other DNA
aagaatctcgcccaggtgaa

SEQ ID NO: 236 - DNA, 21 bp, synthetic construct
  source 1..21: mol_type = other DNA
tgcttgtctcttggtctcacc

SEQ ID NO: 237 - DNA, 21 bp, synthetic construct
  source 1..21: mol_type = other DNA
tggaactgctggagaacaagg

SEQ ID NO: 238 - DNA, 23 bp, synthetic construct
  source 1..23: mol_type = other DNA
agaccctgaaattaccgatttgg

SEQ ID NO: 239 - DNA, 20 bp, synthetic construct
  source 1..20: mol_type = other DNA
tacttcccctgatgccactg

SEQ ID NO: 240 - DNA, 24 bp, synthetic construct
  source 1..24: mol_type = other DNA
atgtcaacaattttagagcagagc

SEQ ID NO: 241 - DNA, 23 bp, synthetic construct
  source 1..23: mol_type = other DNA
ggcccaaagtggatattaatgcc

SEQ ID NO: 242 - DNA, 19 bp, synthetic construct
  source 1..19: mol_type = other DNA
ttgggcagcttcacatccc

SEQ ID NO: 243 - DNA, 20 bp, synthetic construct
  source 1..20: mol_type = other DNA
ctgtgtgctaccagtgcaag

SEQ ID NO: 244 - DNA, 19 bp, synthetic construct
  source 1..19: mol_type = other DNA
cctgagccaagggctcttt

SEQ ID NO: 245 - DNA, 19 bp, synthetic construct
  source 1..19: mol_type = other DNA
cagctgtggaggctgtgac

SEQ ID NO: 246 - DNA, 20 bp, synthetic construct
  source 1..20: mol_type = other DNA
tagagcaggaacaggctgga

SEQ ID NO: 247 - DNA, 20 bp, synthetic construct
  source 1..20: mol_type = other DNA
tggctcagggcgtctttttc

SEQ ID NO: 248 - DNA, 22 bp, synthetic construct
  source 1..22: mol_type = other DNA
agtgcagtttggctcagacata

SEQ ID NO: 249 - DNA, 26 bp, synthetic construct
  source 1..26: mol_type = other DNA
agagtgtttgctgtataattggactt

SEQ ID NO: 250 - DNA, 20 bp, synthetic construct
  source 1..20: mol_type = other DNA
tactgcatggtttgcccaag

SEQ ID NO: 251 - DNA, 24 bp, synthetic construct
  source 1..24: mol_type = other DNA
tgcaaggtgtaggaagatttgttt

SEQ ID NO: 252 - DNA, 29 bp, synthetic construct
  source 1..29: mol_type = other DNA
ataggagtaaaagcaaaaacaaataaacc

SEQ ID NO: 253 - DNA, 24 bp, synthetic construct
  source 1..24: mol_type = other DNA
ccaaggttgtcaccaggaattaac

SEQ ID NO: 254 - DNA, 20 bp, synthetic construct
  source 1..20: mol_type = other DNA
ggtcttgtggccaggagtta

SEQ ID NO: 255 - DNA, 21 bp, synthetic construct
  source 1..21: mol_type = other DNA
cctttttcttcccggatgagc

SEQ ID NO: 256 - DNA, 20 bp, synthetic construct
  source 1..20: mol_type = other DNA
cacaggcataggaggtcctg

SEQ ID NO: 257 - DNA, 24 bp, synthetic construct
  source 1..24: mol_type = other DNA
agatggcagtgaagataatctgct

SEQ ID NO: 258 - DNA, 25 bp, synthetic construct
  source 1..25: mol_type = other DNA
tggaaaattagattaggtcgcgaag

SEQ ID NO: 259 - DNA, 21 bp, synthetic construct
  source 1..21: mol_type = other DNA
cactgttccctgagcctactc

SEQ ID NO: 260 - DNA, 22 bp, synthetic construct
  source 1..22: mol_type = other DNA
acgtgtgtagaaggttctctgt

SEQ ID NO: 261 - DNA, 23 bp, synthetic construct
  source 1..23: mol_type = other DNA
cccacagtcactttgcagaaaat

SEQ ID NO: 262 - DNA, 20 bp, synthetic construct
  source 1..20: mol_type = other DNA
accaagatattgggctggct

SEQ ID NO: 263 - DNA, 24 bp, synthetic construct
  source 1..24: mol_type = other DNA
tctgtaaatgttcttagcctgcct

SEQ ID NO: 264 - DNA, 22 bp, synthetic construct
  source 1..22: mol_type = other DNA
gtgcctgggatattagggttgt

SEQ ID NO: 265 - DNA, 27 bp, synthetic construct
  source 1..27: mol_type = other DNA
cacacacagtaaagaaaatgacgaaag

SEQ ID NO: 266 - DNA, 20 bp, synthetic construct
  source 1..20: mol_type = other DNA
tgctgatcctgtccttctcg

SEQ ID NO: 267 - DNA, 20 bp, synthetic construct
  source 1..20: mol_type = other DNA
gcgagcagatcaagaccctc

SEQ ID NO: 268 - DNA, 20 bp, synthetic construct
  source 1..20: mol_type = other DNA
ccaggttctgcctcacagtc

SEQ ID NO: 269 - DNA, 19 bp, synthetic construct
  source 1..19: mol_type = other DNA
gcagctggacagcatcgtg

SEQ ID NO: 270 - DNA, 24 bp, synthetic construct
  source 1..24: mol_type = other DNA
catcacaaactcattctcagcagt

SEQ ID NO: 271 - DNA, 20 bp, synthetic construct
  source 1..20: mol_type = other DNA
tgaggtcaaggcccagtatg

SEQ ID NO: 272 - DNA, 19 bp, synthetic construct
  source 1..19: mol_type = other DNA
ctcatgcttggtgttgcgg

SEQ ID NO: 273 - DNA, 18 bp, synthetic construct
  source 1..18: mol_type = other DNA
cctcggtggaggtcttgg

SEQ ID NO: 274 - DNA, 20 bp, synthetic construct
  source 1..20: mol_type = other DNA
ctgcttgcactgaagccaga

SEQ ID NO: 275 - DNA, 21 bp, synthetic construct
  source 1..21: mol_type = other DNA
gcttcaaatcagccttcaggt

SEQ ID NO: 276 - DNA, 25 bp, synthetic construct
  source 1..25: mol_type = other DNA
tctggaggtagttagaaccaaaaca

SEQ ID NO: 277 - DNA, 21 bp, synthetic construct
  source 1..21: mol_type = other DNA
caaacgtccaggaggatcatg

SEQ ID NO: 278 - DNA, 18 bp, synthetic construct
  source 1..18: mol_type = other DNA
cggtagcaggagatgcca

SEQ ID NO: 279 - DNA, 18 bp, synthetic construct
  source 1..18: mol_type = other DNA
tgctaccgcggcctcacc

SEQ ID NO: 280 - DNA, 20 bp, synthetic construct
  source 1..20: mol_type = other DNA
tgaccgacacggtggtgatg

SEQ ID NO: 281 - DNA, 21 bp, synthetic construct
  source 1..21: mol_type = other DNA
gccaagtgccagaactccaag

SEQ ID NO: 282 - DNA, 18 bp, synthetic construct
  source 1..18: mol_type = other DNA
ggcaggccatgtcctgct

SEQ ID NO: 283 - DNA, 19 bp, synthetic construct
  source 1..19: mol_type = other DNA
cctggtaccgcagcaagtg

SEQ ID NO: 284 - DNA, 20 bp, synthetic construct
  source 1..20: mol_type = other DNA
ggcacttggcattctccacc

SEQ ID NO: 285 - DNA, 20 bp, synthetic construct
  source 1..20: mol_type = other DNA
agcagctgccggaaatgtta

SEQ ID NO: 286 - DNA, 21 bp, synthetic construct
  source 1..21: mol_type = other DNA
gggtctttcaaagtgcaggag

SEQ ID NO: 287 - DNA, 20 bp, synthetic construct
  source 1..20: mol_type = other DNA
tcaagtccctcaacagcaga

SEQ ID NO: 288 - DNA, 20 bp, synthetic construct
  source 1..20: mol_type = other DNA
tccaggttactctggcagca

SEQ ID NO: 289 - DNA, 21 bp, synthetic construct
  source 1..21: mol_type = other DNA
gagatccgcattctccaatcc

SEQ ID NO: 290 - DNA, 20 bp, synthetic construct
  source 1..20: mol_type = other DNA
ctacgggtggcaatgtcatc

SEQ ID NO: 291 - DNA, 22 bp, synthetic construct
  source 1..22: mol_type = other DNA
aggtgttgaagctgtgaatgtc

SEQ ID NO: 292 - DNA, 20 bp, synthetic construct
  source 1..20: mol_type = other DNA
cagtgctcaccaccaggttc

SEQ ID NO: 293 - DNA, 19 bp, synthetic construct
  source 1..19: mol_type = other DNA
gccgagtcctggtatcgca

SEQ ID NO: 294 - DNA, 19 bp, synthetic construct
  source 1..19: mol_type = other DNA
cattctccacctcggctgt

SEQ ID NO: 295 - DNA, 21 bp, synthetic construct
  source 1..21: mol_type = other DNA
ctggcttttccccttgtgttc

SEQ ID NO: 296 - DNA, 19 bp, synthetic construct
  source 1..19: mol_type = other DNA
gggctgaaggctgagacag

SEQ ID NO: 297 - DNA, 21 bp, synthetic construct
  source 1..21: mol_type = other DNA
gctggctacatcgagactctg

SEQ ID NO: 298 - DNA, 26 bp, synthetic construct
  source 1..26: mol_type = other DNA
tcgaagtgctacttcttcttcatact

SEQ ID NO: 299 - DNA, 20 bp, synthetic construct
  source 1..20: mol_type = other DNA
ccaagtgccagaactccaag

SEQ ID NO: 300 - DNA, 20 bp, synthetic construct
  source 1..20: mol_type = other DNA
atcaggcaggccatgtcttg

SEQ ID NO: 301 - DNA, 19 bp, synthetic construct
  source 1..19: mol_type = other DNA
ccagctgaacaccacctgt

SEQ ID NO: 302 - DNA, 20 bp, synthetic construct
  source 1..20: mol_type = other DNA
agcgatgtgctgctgttttc

SEQ ID NO: 303 - DNA, 22 bp, synthetic construct
  source 1..22: mol_type = other DNA
cacagcagagaacgagtttgtg

SEQ ID NO: 304 - DNA, 20 bp, synthetic construct
  source 1..20: mol_type = other DNA
cggatctcctcctcgtacag

SEQ ID NO: 305 - DNA, 22 bp, synthetic construct
  source 1..22: mol_type = other DNA
tgcactgaatggcatgtgaatg

SEQ ID NO: 306 - DNA, 21 bp, synthetic construct
  source 1..21: mol_type = other DNA
aggatgcatctccctagcatg

SEQ ID NO: 307 - DNA, 20 bp, synthetic construct
  source 1..20: mol_type = other DNA
agaagactccatggctgctc

SEQ ID NO: 308 - DNA, 21 bp, synthetic construct
  source 1..21: mol_type = other DNA
ccagaaggcaatgcacccata

SEQ ID NO: 309 - DNA, 25 bp, synthetic construct
  source 1..25: mol_type = other DNA
tcctacttagctccaaaatacctgt

SEQ ID NO: 310 - DNA, 20 bp, synthetic construct
  source 1..20: mol_type = other DNA
tggctggatggttaggatgg

SEQ ID NO: 311 - DNA, 20 bp, synthetic construct
  source 1..20: mol_type = other DNA
tcagctgatgccaccagatc

SEQ ID NO: 312 - DNA, 20 bp, synthetic construct
  source 1..20: mol_type = other DNA
aacggtgactcagggtcact

SEQ ID NO: 313 - DNA, 20 bp, synthetic construct
  source 1..20: mol_type = other DNA
ttggtgagctcccgaaagaa

SEQ ID NO: 314 - DNA, 21 bp, synthetic construct
  source 1..21: mol_type = other DNA
ggcaaagaggctaaaattggc

SEQ ID NO: 315 - DNA, 20 bp, synthetic construct
  source 1..20: mol_type = other DNA
ttgccccattgcataagcca

SEQ ID NO: 316 - DNA, 19 bp, synthetic construct
  source 1..19: mol_type = other DNA
cttggcatggagctcacct

SEQ ID NO: 317 - DNA, 25 bp, synthetic construct
  source 1..25: mol_type = other DNA
cactaatctcatggcagaatatcgg

SEQ ID NO: 318 - DNA, 19 bp, synthetic construct
  source 1..19: mol_type = other DNA
ctgaccagggagcttggtg

SEQ ID NO: 319 - DNA, 21 bp, synthetic construct
  source 1..21: mol_type = other DNA
ggtccatgacccagaaccttc

SEQ ID NO: 320 - DNA, 20 bp, synthetic construct
  source 1..20: mol_type = other DNA
catgattccaaccccaggca

SEQ ID NO: 321 - DNA, 20 bp, synthetic construct
  source 1..20: mol_type = other DNA
cgtgaagacactcactggca

SEQ ID NO: 322 - DNA, 19 bp, synthetic construct
  source 1..19: mol_type = other DNA
ttccagctgctttccggca

SEQ ID NO: 323 - DNA, 21 bp, synthetic construct
  source 1..21: mol_type = other DNA
gtacagtccaccatctccagt

SEQ ID NO: 324 - DNA, 20 bp, synthetic construct
  source 1..20: mol_type = other DNA
tggaactgaagccacctcca

SEQ ID NO: 325 - DNA, 19 bp, synthetic construct
  source 1..19: mol_type = other DNA
caggctgccattgctgatg

SEQ ID NO: 326 - DNA, 21 bp, synthetic construct
  source 1..21: mol_type = other DNA
tcagctcctggtactccttca

SEQ ID NO: 327 - DNA, 20 bp, synthetic construct
  source 1..20: mol_type = other DNA
taaagtgctgcctccagctc

SEQ ID NO: 328 - DNA, 20 bp, synthetic construct
  source 1..20: mol_type = other DNA
aagcatccatacccagctct

SEQ ID NO: 329 - DNA, 20 bp, synthetic construct
  source 1..20: mol_type = other DNA
atacctgccccatctgagca

SEQ ID NO: 330 - DNA, 24 bp, synthetic construct
  source 1..24: mol_type = other DNA
gcctcagagagaacaattttggag

SEQ ID NO: 331 - DNA, 26 bp, synthetic construct
  source 1..26: mol_type = other DNA
tgaggctgagcattataagaaaatga

SEQ ID NO: 332 - DNA, 27 bp, synthetic construct
  source 1..27: mol_type = other DNA
gggttcacaatacttttaacttaggga

SEQ ID NO: 333 - DNA, 22 bp, synthetic construct
  source 1..22: mol_type = other DNA
ggagctgatgaatgtcaagctg

SEQ ID NO: 334 - DNA, 24 bp, synthetic construct
  source 1..24: mol_type = other DNA
actgtactacagagacgttgactt

SEQ ID NO: 335 - DNA, 18 bp, synthetic construct
  source 1..18: mol_type = other DNA
tgcaaattgaccccgcca

SEQ ID NO: 336 - DNA, 21 bp, synthetic construct
  source 1..21: mol_type = other DNA
agaaccttgttctgctgctct

SEQ ID NO: 337 - DNA, 19 bp, synthetic construct
  source 1..19: mol_type = other DNA
ggtgcatgtggaggagctg

SEQ ID NO: 338 - DNA, 19 bp, synthetic construct
  source 1..19: mol_type = other DNA
gccaccaaagccatagctg

SEQ ID NO: 339 - DNA, 27 bp, synthetic construct
  source 1..27: mol_type = other DNA
tgcaatttggaaacctactgtattgaa

SEQ ID NO: 340 - DNA, 21 bp, synthetic construct
  source 1..21: mol_type = other DNA
tctttggccttagtggccttt

SEQ ID NO: 341 - DNA, 23 bp, synthetic construct
  source 1..23: mol_type = other DNA
gggttgacagagaaagcaaagtg

SEQ ID NO: 342 - DNA, 22 bp, synthetic construct
  source 1..22: mol_type = other DNA
actcgtttacaagcaactaccc

SEQ ID NO: 343 - DNA, 20 bp, synthetic construct
  source 1..20: mol_type = other DNA
cagctgcataaggactgccc

SEQ ID NO: 344 - DNA, 19 bp, synthetic construct
  source 1..19: mol_type = other DNA
ctgctgtagccgctgctca

SEQ ID NO: 345 - DNA, 21 bp, synthetic construct
  source 1..21: mol_type = other DNA
gccgaggacttcttcttcacc

SEQ ID NO: 346 - DNA, 24 bp, synthetic construct
  source 1..24: mol_type = other DNA
tttctctttgatctcctctttgct

SEQ ID NO: 347 - DNA, 19 bp, synthetic construct
  source 1..19: mol_type = other DNA
ccgcagagcctgctcactc

SEQ ID NO: 348 - DNA, 18 bp, synthetic construct
  source 1..18: mol_type = other DNA
cctccaggcccggcttct

SEQ ID NO: 349 - DNA, 21 bp, synthetic construct
  source 1..21: mol_type = other DNA
agacttttcctgccacgactt

SEQ ID NO: 350 - DNA, 20 bp, synthetic construct
  source 1..20: mol_type = other DNA
ttggcatccagggaagtgtc

SEQ ID NO: 351 - DNA, 18 bp, synthetic construct
  source 1..18: mol_type = other DNA
gccggcctcgctttccca

SEQ ID NO: 352 - DNA, 18 bp, synthetic construct
  source 1..18: mol_type = other DNA
ggctgtcgctggcgaaca

SEQ ID NO: 353 - DNA, 20 bp, synthetic construct
  source 1..20: mol_type = other DNA
gattggacaggaacgagctg

SEQ ID NO: 354 - DNA, 27 bp, synthetic construct
  source 1..27: mol_type = other DNA
gctcctcagttcttttgatttctttct

SEQ ID NO: 355 - DNA, 20 bp, synthetic construct
  source 1..20: mol_type = other DNA
atccggaccctggaacaaga

SEQ ID NO: 356 - DNA, 23 bp, synthetic construct
  source 1..23: mol_type = other DNA
tcttggctttgttgttagggttg

SEQ ID NO: 357 - DNA, 21 bp, synthetic construct
  source 1..21: mol_type = other DNA
cctggtgaggcgagagttatg

SEQ ID NO: 358 - DNA, 19 bp, synthetic construct
  source 1..19: mol_type = other DNA
acagccggaaccgcttatc

SEQ ID NO: 359 - DNA, 20 bp, synthetic construct
  source 1..20: mol_type = other DNA
gagaggtgatcctggtgctc

SEQ ID NO: 360 - DNA, 19 bp, synthetic construct
  source 1..19: mol_type = other DNA
gatatgctgcgccttgtgc

SEQ ID NO: 361 - DNA, 23 bp, synthetic construct
  source 1..23: mol_type = other DNA
gcctaataaacaacagcctcctg

SEQ ID NO: 362 - DNA, 20 bp, synthetic construct
  source 1..20: mol_type = other DNA
tggcccaagctgtactgaaa

SEQ ID NO: 363 - DNA, 22 bp, synthetic construct
  source 1..22: mol_type = other DNA
cctgaggcataggtactgttgt

SEQ ID NO: 364 - DNA, 21 bp, synthetic construct
  source 1..21: mol_type = other DNA
ctggtaggtcaagctgtagca

SEQ ID NO: 365 - DNA, 24 bp, synthetic construct
  source 1..24: mol_type = other DNA
ggttgctttcttgccaaaataagt

SEQ ID NO: 366 - DNA, 26 bp, synthetic construct
  source 1..26: mol_type = other DNA
ccaaaatagaggctctgacttagaag

SEQ ID NO: 367 - DNA, 27 bp, synthetic construct
  source 1..27: mol_type = other DNA
ggtctagcaatacactgtttacaagag

SEQ ID NO: 368 - DNA, 27 bp, synthetic construct
  source 1..27: mol_type = other DNA
tctgagattcaaagagctacatttttg

SEQ ID NO: 369 - DNA, 18 bp, synthetic construct
  source 1..18: mol_type = other DNA
gcgctgacgggtgtggac

SEQ ID NO: 370 - DNA, 22 bp, synthetic construct
  source 1..22: mol_type = other DNA
aagtgaggcgcgatataaaccc

SEQ ID NO: 371 - DNA, 20 bp, synthetic construct
  source 1..20: mol_type = other DNA
tgcattgcacgttggatgag

SEQ ID NO: 372 - DNA, 27 bp, synthetic construct
  source 1..27: mol_type = other DNA
cttccttgtttctcccaaataaaatca

SEQ ID NO: 373 - DNA, 29 bp, synthetic construct
  source 1..29: mol_type = other DNA
gcttaatagggaacatacatacagctatg

SEQ ID NO: 374 - DNA, 29 bp, synthetic construct
  source 1..29: mol_type = other DNA
ggacccacactaaaaatttttaagtcaat

SEQ ID NO: 375 - DNA, 24 bp, synthetic construct
  source 1..24: mol_type = other DNA
tcccaggccttactgtaaaactag

SEQ ID NO: 376 - DNA, 20 bp, synthetic construct
  source 1..20: mol_type = other DNA
atttcccctactgccccagc

SEQ ID NO: 377 - DNA, 21 bp, synthetic construct
  source 1..21: mol_type = other DNA
ctctggtggcagactttgtag

SEQ ID NO: 378 - DNA, 20 bp, synthetic construct
  source 1..20: mol_type = other DNA
aagcgggtctcgaactcatc

SEQ ID NO: 379 - DNA, 20 bp, synthetic construct
  source 1..20: mol_type = other DNA
ttaacagtgggctgcttgga

SEQ ID NO: 380 - DNA, 20 bp, synthetic construct
  source 1..20: mol_type = other DNA
tgcccctagttcttgccatt

SEQ ID NO: 381 - DNA, 20 bp, synthetic construct
  source 1..20: mol_type = other DNA
gtgcttccttccccaggtat

SEQ ID NO: 382 - DNA, 21 bp, synthetic construct
  source 1..21: mol_type = other DNA
agcagggcattttggtaacct

SEQ ID NO: 383 - DNA, 22 bp, synthetic construct
  source 1..22: mol_type = other DNA
cagagcccttctatttgtccca

SEQ ID NO: 384 - DNA, 25 bp, synthetic construct
  source 1..25: mol_type = other DNA
cagatattcagggattagctcctca

SEQ ID NO: 385 - DNA, 20 bp, synthetic construct
  source 1..20: mol_type = other DNA
ctagctgctccaagggttga

SEQ ID NO: 386 - DNA, 27 bp, synthetic construct
  source 1..27: mol_type = other DNA
cactaagagaacacatgtaactcatgg

SEQ ID NO: 387 - DNA, 20 bp, synthetic construct
  source 1..20: mol_type = other DNA
ttggcaacgcatgcggtata

SEQ ID NO: 388 - DNA, 21 bp, synthetic construct
  source 1..21: mol_type = other DNA
tgttccaggtaatccatggct

SEQ ID NO: 389 - DNA, 23 bp, synthetic construct
  source 1..23: mol_type = other DNA
tggaacaaagtgcagattagtgc

SEQ ID NO: 390 - DNA, 26 bp, synthetic construct
  source 1..26: mol_type = other DNA
agctaaagaaaatactcttgatcggg

SEQ ID NO: 391 - DNA, 22 bp, synthetic construct
  source 1..22: mol_type = other DNA
ggtaaacaagtcactggcacag

SEQ ID NO: 392 - DNA, 20 bp, synthetic construct
  source 1..20: mol_type = other DNA
tcagtgactgggaacaggtc

SEQ ID NO: 393 - DNA, 26 bp, synthetic construct
  source 1..26: mol_type = other DNA
agaagagagaaacaactcagatttgc

SEQ ID NO: 394 - DNA, 22 bp, synthetic construct
  source 1..22: mol_type = other DNA
cctctccagggaaatcttgtcg

SEQ ID NO: 395 - DNA, 21 bp, synthetic construct
  source 1..21: mol_type = other DNA
ggcagctcaactccatgaaga

SEQ ID NO: 396 - DNA, 22 bp, synthetic construct
  source 1..22: mol_type = other DNA
tggagaatgtgtagctcacagg

SEQ ID NO: 397 - DNA, 19 bp, synthetic construct
  source 1..19: mol_type = other DNA
ggagcatctgtctgccact

SEQ ID NO: 398 - DNA, 24 bp, synthetic construct
  source 1..24: mol_type = other DNA
tcaggcatcccaacattttaaact

SEQ ID NO: 399 - DNA, 21 bp, synthetic construct
  source 1..21: mol_type = other DNA
tctcacctctgcctgcttatg

SEQ ID NO: 400 - DNA, 26 bp, synthetic construct
  source 1..26: mol_type = other DNA
gcctgtttaattcactgctgtattct

SEQ ID NO: 401 - DNA, 18 bp, synthetic construct
  source 1..18: mol_type = other DNA
cttgttgccgaggctgga

SEQ ID NO: 402 - DNA, 22 bp, synthetic construct
  source 1..22: mol_type = other DNA
gagtggtggtggtcatctgtaa

SEQ ID NO: 403 - DNA, 22 bp, synthetic construct
  source 1..22: mol_type = other DNA
agttctttccagtagccatggg

SEQ ID NO: 404 - DNA, 24 bp, synthetic construct
  source 1..24: mol_type = other DNA
tgtaggatagcgcaattgtcaaaa

SEQ ID NO: 405 - DNA, 23 bp, synthetic construct
  source 1..23: mol_type = other DNA
ggtgcacagagtaaaaaggagga

SEQ ID NO: 406 - DNA, 22 bp, synthetic construct
  source 1..22: mol_type = other DNA
tgagtatttgaggccgaaggaa

SEQ ID NO: 407 - DNA, 20 bp, synthetic construct
  source 1..20: mol_type = other DNA
cctttgcaaagctgcaggga

SEQ ID NO: 408 - DNA, 20 bp, synthetic construct
  source 1..20: mol_type = other DNA
gcgcacggatcactgtactt

SEQ ID NO: 409 - DNA, 22 bp, synthetic construct
  source 1..22: mol_type = other DNA
acttaatagcactgtgcaggga

SEQ ID NO: 410 - DNA, 23 bp, synthetic construct
  source 1..23: mol_type = other DNA
gcatgaactttacaaaccagcca

SEQ ID NO: 411 - DNA, 21 bp, synthetic construct
  source 1..21: mol_type = other DNA
tgtccccaaccatcccaatag

SEQ ID NO: 412 - DNA, 21 bp, synthetic construct
  source 1..21: mol_type = other DNA
agactcccacccttaaactgg

SEQ ID NO: 413 - DNA, 25 bp, synthetic construct
  source 1..25: mol_type = other DNA
acaatgcaaaactgttagaccagta

SEQ ID NO: 414 - DNA, 29 bp, synthetic construct
  source 1..29: mol_type = other DNA
agacaacacttaaatacacattacctgtt

SEQ ID NO: 415 - DNA, 18 bp, synthetic construct
  source 1..18: mol_type = other DNA
gctggaggcgtccgagcc

SEQ ID NO: 416 - DNA, 24 bp, synthetic construct
  source 1..24: mol_type = other DNA
ggtactccacctccgagaagacag

SEQ ID NO: 417 - DNA, 27 bp, synthetic construct
  source 1..27: mol_type = other DNA
tgaacgtatttcagaaatcactgtgtt

SEQ ID NO: 418 - DNA, 22 bp, synthetic construct
  source 1..22: mol_type = other DNA
tctattgccttcagtcttgacg

SEQ ID NO: 419 - DNA, 25 bp, synthetic construct
  source 1..25: mol_type = other DNA
tggtcatatgaggtcctaatgaagt

SEQ ID NO: 420 - DNA, 22 bp, synthetic construct
  source 1..22: mol_type = other DNA
cctctttgaaccgacaagcttt

SEQ ID NO: 421 - DNA, 26 bp, synthetic construct
  source 1..26: mol_type = other DNA
ccatttgatcagggaattttctacac

SEQ ID NO: 422 - DNA, 23 bp, synthetic construct
  source 1..23: mol_type = other DNA
tgtgggaatttaaatagcggctt

SEQ ID NO: 423 - DNA, 18 bp, synthetic construct
  source 1..18: mol_type = other DNA
cctctgccgacctcaggg

SEQ ID NO: 424 - DNA, 19 bp, synthetic construct
  source 1..19: mol_type = other DNA
gaatagcagcccttgcgct

SEQ ID NO: 425 - DNA, 24 bp, synthetic construct
  source 1..24: mol_type = other DNA
tcatgaagtgtccctgtgaaattg

SEQ ID NO: 426 - DNA, 24 bp, synthetic construct
  source 1..24: mol_type = other DNA
atcttcaacctactgcttacagtc

SEQ ID NO: 427 - DNA, 25 bp, synthetic construct
  source 1..25: mol_type = other DNA
ttgaaacagaaaatacctgggaatg

SEQ ID NO: 428 - DNA, 25 bp, synthetic construct
  source 1..25: mol_type = other DNA
tgcatcaaaatgaaaattccaaggc

SEQ ID NO: 429 - DNA, 23 bp, synthetic construct
  source 1..23: mol_type = other DNA
ggatatgagctggttgacactga

SEQ ID NO: 430 - DNA, 20 bp, synthetic construct
  source 1..20: mol_type = other DNA
gcacacttctccctcacagt

SEQ ID NO: 431 - DNA, 24 bp, synthetic construct
  source 1..24: mol_type = other DNA
cccctttaagtgctttaacaggat

SEQ ID NO: 432 - DNA, 28 bp, synthetic construct
  source 1..28: mol_type = other DNA
gccttttatttgggactgtgtagttatc

SEQ ID NO: 433 - DNA, 22 bp, synthetic construct
  source 1..22: mol_type = other DNA
cggagctggaattccataactc

SEQ ID NO: 434 - DNA, 20 bp, synthetic construct
  source 1..20: mol_type = other DNA
tcgctgccatgttgatttcc

SEQ ID NO: 435 - DNA, 22 bp, synthetic construct
  source 1..22: mol_type = other DNA
gaaagatatgggccttggagga

SEQ ID NO: 436 - DNA, 20 bp, synthetic construct
  source 1..20: mol_type = other DNA
tactttcgctcctgctccac

SEQ ID NO: 437 - DNA, 20 bp, synthetic construct
  source 1..20: mol_type = other DNA
ggaaagaggaggagctctcg

SEQ ID NO: 438 - DNA, 20 bp, synthetic construct
  source 1..20: mol_type = other DNA
agctccacatcgatgctctc

SEQ ID NO: 439 - DNA, 21 bp, synthetic construct
  source 1..21: mol_type = other DNA
caggagtacgtggtcaaggag

SEQ ID NO: 440 - DNA, 18 bp, synthetic construct
  source 1..18: mol_type = other DNA
caggaggagcacctctgc

SEQ ID NO: 441 - DNA, 20 bp, synthetic construct
  source 1..20: mol_type = other DNA
acaccagacagtgacactgc

SEQ ID NO: 442 - DNA, 25 bp, synthetic construct
  source 1..25: mol_type = other DNA
gtctcatgagcagagtaactactga

SEQ ID NO: 443 - DNA, 21 bp, synthetic construct
  source 1..21: mol_type = other DNA
tgcggattgaacctgagaaga

SEQ ID NO: 444 - DNA, 20 bp, synthetic construct
  source 1..20: mol_type = other DNA
tgcaggaggtgctcgtattt

SEQ ID NO: 445 - DNA, 20 bp, synthetic construct
  source 1..20: mol_type = other DNA
acagctacaccctgcagaag

SEQ ID NO: 446 - DNA, 18 bp, synthetic construct
  source 1..18: mol_type = other DNA
ccaggctgtcagccagag

SEQ ID NO: 447 - DNA, 25 bp, synthetic construct
  source 1..25: mol_type = other DNA
tgattattcaccacgccaatatctg

SEQ ID NO: 448 - DNA, 20 bp, synthetic construct
  source 1..20: mol_type = other DNA
accgtacaagcagactccag

SEQ ID NO: 449 - DNA, 20 bp, synthetic construct
  source 1..20: mol_type = other DNA
gcctcagacaccaccatgac

SEQ ID NO: 450 - DNA, 18 bp, synthetic construct
  source 1..18: mol_type = other DNA
cggcacacggtggtctgg

SEQ ID NO: 451 - DNA, 26 bp, synthetic construct
  source 1..26: mol_type = other DNA
cacactcccatttctctactttaaca

SEQ ID NO: 452 - DNA, 27 bp, synthetic construct
  source 1..27: mol_type = other DNA
gaaattgcttcttttacagacttcaga

SEQ ID NO: 453 - DNA, 20 bp, synthetic construct
  source 1..20: mol_type = other DNA
ttgctgtcagcctacctgct

SEQ ID NO: 454 - DNA, 19 bp, synthetic construct
  source 1..19: mol_type = other DNA
caagttgggtggcagcaag

SEQ ID NO: 455 - DNA, 20 bp, synthetic construct
  source 1..20: mol_type = other DNA
gggtcgagcctgtttgtttc

SEQ ID NO: 456 - DNA, 22 bp, synthetic construct
  source 1..22: mol_type = other DNA
tcctccttgcattccagagaat

SEQ ID NO: 457 - DNA, 20 bp, synthetic construct
  source 1..20: mol_type = other DNA
tgaaccaggtcctgaacgag

SEQ ID NO: 458 - DNA, 19 bp, synthetic construct
  source 1..19: mol_type = other DNA
aggactgcagctgctctga

SEQ ID NO: 459 - DNA, 19 bp, synthetic construct
  source 1..19: mol_type = other DNA
caacgccctggagatcgag

SEQ ID NO: 460 - DNA, 19 bp, synthetic construct
  source 1..19: mol_type = other DNA
tctgccagctgagactcca

SEQ ID NO: 461 - DNA, 20 bp, synthetic construct
  source 1..20: mol_type = other DNA
aatgctgacctggaggtgaa

SEQ ID NO: 462 - DNA, 20 bp, synthetic construct
  source 1..20: mol_type = other DNA
atggtggtggccatgatctt

SEQ ID NO: 463 - DNA, 21 bp, synthetic construct
  source 1..21: mol_type = other DNA
gacataaagacacggctggag

SEQ ID NO: 464 - DNA, 20 bp, synthetic construct
  source 1..20: mol_type = other DNA
tgtggaaattgctgctgcta

SEQ ID NO: 465 - DNA, 21 bp, synthetic construct
  source 1..21: mol_type = other DNA
acatcatttgcatggctggag

SEQ ID NO: 466 - DNA, 21 bp, synthetic construct
  source 1..21: mol_type = other DNA
tctgggtgagcataggaagga

SEQ ID NO: 467 - DNA, 19 bp, synthetic construct
  source 1..19: mol_type = other DNA
gtgggccctgcaattcctt

SEQ ID NO: 468 - DNA, 22 bp, synthetic construct
  source 1..22: mol_type = other DNA
tgatgttttcaggccccttttg

SEQ ID NO: 469 - DNA, 20 bp, synthetic construct
  source 1..20: mol_type = other DNA
ccttaacatcgaggtggacg

SEQ ID NO: 470 - DNA, 23 bp, synthetic construct
  source 1..23: mol_type = other DNA
agctcctccatctgcatattgaa

SEQ ID NO: 471 - DNA, 21 bp, synthetic construct
  source 1..21: mol_type = other DNA
cagctgcctctccaaaaccta

SEQ ID NO: 472 - DNA, 22 bp, synthetic construct
  source 1..22: mol_type = other DNA
gtcgttaaggaactgcatggtt

SEQ ID NO: 473 - DNA, 21 bp, synthetic construct
  source 1..21: mol_type = other DNA
aggagcttaaccaacaggtgg

SEQ ID NO: 474 - DNA, 18 bp, synthetic construct
  source 1..18: mol_type = other DNA
agtccctcaggctgtgct

SEQ ID NO: 475 - DNA, 18 bp, synthetic construct
  source 1..18: mol_type = other DNA
caccacctgtgtgccctc

SEQ ID NO: 476 - DNA, 19 bp, synthetic construct
  source 1..19: mol_type = other DNA
gccctccaggatccactgg

SEQ ID NO: 477 - DNA, 19 bp, synthetic construct
  source 1..19: mol_type = other DNA
gtaccaggtgctgctggac

SEQ ID NO: 478 - DNA, 19 bp, synthetic construct
  source 1..19: mol_type = other DNA
caggtggtgcaggaaggag

SEQ ID NO: 479 - DNA, 21 bp, synthetic construct
  source 1..21: mol_type = other DNA
cgcaggatcctggatgatctc

SEQ ID NO: 480 - DNA, 20 bp, synthetic construct
  source 1..20: mol_type = other DNA
gcatcgaagggaaccgactt

SEQ ID NO: 481 - DNA, 20 bp, synthetic construct
  source 1..20: mol_type = other DNA
gggactccctggaaaacacg

SEQ ID NO: 482 - DNA, 19 bp, synthetic construct
  source 1..19: mol_type = other DNA
ttctgccgctccaggtcag

SEQ ID NO: 483 - DNA, 19 bp, synthetic construct
  source 1..19: mol_type = other DNA
gtatccagctcggagcagc

SEQ ID NO: 484 - DNA, 18 bp, synthetic construct
  source 1..18: mol_type = other DNA
cgggcctcgctctctgtc

SEQ ID NO: 485 - DNA, 21 bp, synthetic construct
  source 1..21: mol_type = other DNA
gcttctcttctgggtctctca

SEQ ID NO: 486 - DNA, 20 bp, synthetic construct
  source 1..20: mol_type = other DNA
gcaggcagagaaacttctgg

SEQ ID NO: 487 - DNA, 20 bp, synthetic construct
  source 1..20: mol_type = other DNA
ctgaaccagcaggtggtgtc

SEQ ID NO: 488 - DNA, 21 bp, synthetic construct
  source 1..21: mol_type = other DNA
cagggtggattccaaagcatc

SEQ ID NO: 489 - DNA, 20 bp, synthetic construct
  source 1..20: mol_type = other DNA
tcatgccttccctgtcttcc

SEQ ID NO: 490 - DNA, 21 bp, synthetic construct
  source 1..21: mol_type = other DNA
agacaatagccatggccatct

SEQ ID NO: 491 - DNA, 19 bp, synthetic construct
  source 1..19: mol_type = other DNA
gcaggtggtatccagctcg

SEQ ID NO: 492 - DNA, 21 bp, synthetic construct
  source 1..21: mol_type = other DNA
tctctgtcagcgtgttttcca

SEQ ID NO: 493 - DNA, 21 bp, synthetic construct
  source 1..21: mol_type = other DNA
gccacaaccaatgcatgtgac

SEQ ID NO: 494 - DNA, 20 bp, synthetic construct
  source 1..20: mol_type = other DNA
tcttctcctcctggttgtgg

SEQ ID NO: 495 - DNA, 20 bp, synthetic construct
  source 1..20: mol_type = other DNA
gagggctccacaggtaagac

SEQ ID NO: 496 - DNA, 20 bp, synthetic construct
  source 1..20: mol_type = other DNA
tgtgcactgggcatttacca

SEQ ID NO: 497 - DNA, 23 bp, synthetic construct
  source 1..23: mol_type = other DNA
ggagatggagaatggctattggt

SEQ ID NO: 498 - DNA, 20 bp, synthetic construct
  source 1..20: mol_type = other DNA
tcccatgaagcccagttcac

SEQ ID NO: 499 - DNA, 20 bp, synthetic construct
  source 1..20: mol_type = other DNA
cctgtcctgacaggggagag

SEQ ID NO: 500 - DNA, 18 bp, synthetic construct
  source 1..18: mol_type = other DNA
cggaacctccctccccag

SEQ ID NO: 501 - DNA, 21 bp, synthetic construct
  source 1..21: mol_type = other DNA
tccggtcatccacttatgtgc

SEQ ID NO: 502 - DNA, 20 bp, synthetic construct
  source 1..20: mol_type = other DNA
acgatatggcaagaggcagt

SEQ ID NO: 503 - DNA, 21 bp, synthetic construct
  source 1..21: mol_type = other DNA
tgctgctgaaaccagaatgtg

SEQ ID NO: 504 - DNA, 23 bp, synthetic construct
  source 1..23: mol_type = other DNA
agaataccggacaagactttctg

SEQ ID NO: 505 - DNA, 18 bp, synthetic construct
  source 1..18: mol_type = other DNA
gagctgcacacccccatc

SEQ ID NO: 506 - DNA, 24 bp, synthetic construct
  source 1..24: mol_type = other DNA
gcagaaggaggttttcaacaagtg

SEQ ID NO: 507 - DNA, 20 bp, synthetic construct
  source 1..20: mol_type = other DNA
tccaagcaacccaaccttca

SEQ ID NO: 508 - DNA, 20 bp, synthetic construct
  source 1..20: mol_type = other DNA
gagctggtctcacagcagct

SEQ ID NO: 509 - DNA, 20 bp, synthetic construct
  source 1..20: mol_type = other DNA
agctaaccagacgcccattg

SEQ ID NO: 510 - DNA, 19 bp, synthetic construct
  source 1..19: mol_type = other DNA
ctgtgtgtgggcaggtgct

SEQ ID NO: 511 - DNA, 22 bp, synthetic construct
  source 1..22: mol_type = other DNA
cacacacagtttggttactgga

SEQ ID NO: 512 - DNA, 20 bp, synthetic construct
  source 1..20: mol_type = other DNA
tggtgaggttgagggtctcc

SEQ ID NO: 513 - DNA, 20 bp, synthetic construct
  source 1..20: mol_type = other DNA
tactttgctcagtgcccgac

SEQ ID NO: 514 - DNA, 24 bp, synthetic construct
  source 1..24: mol_type = other DNA
tcccccattaaaaccaaaggtaag

SEQ ID NO: 515 - DNA, 18 bp, synthetic construct
  source 1..18: mol_type = other DNA
ctgctgcagaccccagtg

SEQ ID NO: 516 - DNA, 18 bp, synthetic construct
  source 1..18: mol_type = other DNA
gcagcagctagggtggca

SEQ ID NO: 517 - DNA, 18 bp, synthetic construct
  source 1..18: mol_type = other DNA
ccctgctgctgcctgcgt

SEQ ID NO: 518 - DNA, 18 bp, synthetic construct
  source 1..18: mol_type = other DNA
gccagggcagtgggctca

SEQ ID NO: 519 - DNA, 20 bp, synthetic construct
  source 1..20: mol_type = other DNA
cctggctcatctctcccttc

SEQ ID NO: 520 - DNA, 20 bp, synthetic construct
  source 1..20: mol_type = other DNA
gggatggtgatgggctcatt

SEQ ID NO: 521 - DNA, 18 bp, synthetic construct
  source 1..18: mol_type = other DNA
gccgccccagctgctgtg

SEQ ID NO: 522 - DNA, 19 bp, synthetic construct
  source 1..19: mol_type = other DNA
gggcggcagcagctggatt

SEQ ID NO: 523 - DNA, 22 bp, synthetic construct
  source 1..22: mol_type = other DNA
actccctcccttgctttctttt

SEQ ID NO: 524 - DNA, 27 bp, synthetic construct
  source 1..27: mol_type = other DNA
ggaaggatattctgtaggctcaaaatg

SEQ ID NO: 525 - DNA, 28 bp, synthetic construct
  source 1..28: mol_type = other DNA
gctgttcgattccttcatattaaagtgt

SEQ ID NO: 526 - DNA, 32 bp, synthetic construct
  source 1..32: mol_type = other DNA
gctatgccagagatgtaaaatttattaggata

SEQ ID NO: 527 - DNA, 26 bp, synthetic construct
  source 1..26: mol_type = other DNA
ccttatccttcttttcaatcaaaccc

SEQ ID NO: 528 - DNA, 18 bp, synthetic construct
  source 1..18: mol_type = other DNA
ctccacagcggcaggatt

SEQ ID NO: 529 - DNA, 22 bp, synthetic construct
  source 1..22: mol_type = other DNA
cacacacagtttggttactgga

SEQ ID NO: 530 - DNA, 20 bp, synthetic construct
  source 1..20: mol_type = other DNA
tggtgaggttgagggtctcc

SEQ ID NO: 531 - DNA, 21 bp, synthetic construct
  source 1..21: mol_type = other DNA
aatacttgcccaaggtcacac

SEQ ID NO: 532 - DNA, 22 bp, synthetic construct
  source 1..22: mol_type = other DNA
aggtaaggaaccgagaaggttg

SEQ ID NO: 533 - DNA, 21 bp, synthetic construct
  source 1..21: mol_type = other DNA
agtttgaggctgcagtgagtt

SEQ ID NO: 534 - DNA, 26 bp, synthetic construct
  source 1..26: mol_type = other DNA
cttccatctttccctttctttctttc

SEQ ID NO: 535 - DNA, 23 bp, synthetic construct
  source 1..23: mol_type = other DNA
gactttaatggaggaggtgaggt

SEQ ID NO: 536 - DNA, 23 bp, synthetic construct
  source 1..23: mol_type = other DNA
cttagttcgatgtcccttcctct

SEQ ID NO: 537 - DNA, 20 bp, synthetic construct
  source 1..20: mol_type = other DNA
tccactgggcaccaacataa

SEQ ID NO: 538 - DNA, 27 bp, synthetic construct
  source 1..27: mol_type = other DNA
gaacatgaaagagaatgagaaacagag

SEQ ID NO: 539 - DNA, 23 bp, synthetic construct
  source 1..23: mol_type = other DNA
gcaggaagaagttgtacctgact

SEQ ID NO: 540 - DNA, 22 bp, synthetic construct
  source 1..22: mol_type = other DNA
gtcctatgtctgtttcctgcct

SEQ ID NO: 541 - DNA, 21 bp, synthetic construct
  source 1..21: mol_type = other DNA
gggcgaaacagagactctgtc

SEQ ID NO: 542 - DNA, 19 bp, synthetic construct
  source 1..19: mol_type = other DNA
ctgggctcaggcaatcctc

SEQ ID NO: 543 - DNA, 21 bp, synthetic construct
  source 1..21: mol_type = other DNA
gcagtgagctgtgattgcatc

SEQ ID NO: 544 - DNA, 20 bp, synthetic construct
  source 1..20: mol_type = other DNA
gaatcgtgcctggcacatag

SEQ ID NO: 545 - DNA, 22 bp, synthetic construct
  source 1..22: mol_type = other DNA
ctggtcacagtctacctcttcc

SEQ ID NO: 546 - DNA, 20 bp, synthetic construct
  source 1..20: mol_type = other DNA
catccgcattgaaactggca

SEQ ID NO: 547 - DNA, 23 bp, synthetic construct
  source 1..23: mol_type = other DNA
ccatgacaccattgtaagagctg

SEQ ID NO: 548 - DNA, 25 bp, synthetic construct
  source 1..25: mol_type = other DNA
ggaaacaaaatcagagaggtagtgt

SEQ ID NO: 549 - DNA, 20 bp, synthetic construct
  source 1..20: mol_type = other DNA
atgaagagggagacgatggg

SEQ ID NO: 550 - DNA, 21 bp, synthetic construct
  source 1..21: mol_type = other DNA
cactctgtgggacattcccta

SEQ ID NO: 551 - DNA, 18 bp, synthetic construct
  source 1..18: mol_type = other DNA
cacttggtggccctgttg

SEQ ID NO: 552 - DNA, 21 bp, synthetic construct
  source 1..21: mol_type = other DNA
ccgatggtggacagagagata

SEQ ID NO: 553 - DNA, 24 bp, synthetic construct
  source 1..24: mol_type = other DNA
gaagaaaagcaagtcctttttggt

SEQ ID NO: 554 - DNA, 19 bp, synthetic construct
  source 1..19: mol_type = other DNA
catgtccaggctggtctcc

SEQ ID NO: 555 - DNA, 19 bp, synthetic construct
  source 1..19: mol_type = other DNA
gagcagcttgctcgggatc

SEQ ID NO: 556 - DNA, 20 bp, synthetic construct
  source 1..20: mol_type = other DNA
ggctaaaggtgggttcctga

SEQ ID NO: 557 - DNA, 24 bp, synthetic construct
  source 1..24: mol_type = other DNA
agaggagggagttaacaagttctc

SEQ ID NO: 558 - DNA, 20 bp, synthetic construct
  source 1..20: mol_type = other DNA
tggatcctggagacaaggca

SEQ ID NO: 559 - DNA, 19 bp, synthetic construct
  source 1..19: mol_type = other DNA
ccaggaagacctccgtgtc

SEQ ID NO: 560 - DNA, 20 bp, synthetic construct
  source 1..20: mol_type = other DNA
gcagcttctcctttcccagt

SEQ ID NO: 561 - DNA, 20 bp, synthetic construct
  source 1..20: mol_type = other DNA
tcttcctcctgtccccagtg

SEQ ID NO: 562 - DNA, 19 bp, synthetic construct
  source 1..19: mol_type = other DNA
cctccaggccagcttctac

SEQ ID NO: 563 - DNA, 19 bp, synthetic construct
  source 1..19: mol_type = other DNA
caagtcctggtggtgccaa

SEQ ID NO: 564 - DNA, 21 bp, synthetic construct
  source 1..21: mol_type = other DNA
ccaatacacccaggccttcag

SEQ ID NO: 565 - DNA, 19 bp, synthetic construct
  source 1..19: mol_type = other DNA
cctgtgatcgcttctggca

SEQ ID NO: 566 - DNA, 20 bp, synthetic construct
  source 1..20: mol_type = other DNA
aggaagaaagggtgggcatc

SEQ ID NO: 567 - DNA, 20 bp, synthetic construct
  source 1..20: mol_type = other DNA
ccttcctcctctgtcccttc

SEQ ID NO: 568 - DNA, 20 bp, synthetic construct
  source 1..20: mol_type = other DNA
gcaaaccagttggtctcagc

SEQ ID NO: 569 - DNA, 20 bp, synthetic construct
  source 1..20: mol_type = other DNA
aaaggaggatggcgagttct

SEQ ID NO: 570 - DNA, 20 bp, synthetic construct
  source 1..20: mol_type = other DNA
ccttggaaggtgtggacgtg

SEQ ID NO: 571 - DNA, 21 bp, synthetic construct
  source 1..21: mol_type = other DNA
ctgggtaactgctggttcctt

SEQ ID NO: 572 - DNA, 20 bp, synthetic construct
  source 1..20: mol_type = other DNA
aactgccagagctggaagtg

SEQ ID NO: 573 - DNA, 20 bp, synthetic construct
  source 1..20: mol_type = other DNA
ctgcgtactcctcactcaga

SEQ ID NO: 574 - DNA, 20 bp, synthetic construct
  source 1..20: mol_type = other DNA
tctcctgtgtctgtcctcca

SEQ ID NO: 575 - DNA, 20 bp, synthetic construct
  source 1..20: mol_type = other DNA
ttaacgagggaaggctcctc

SEQ ID NO: 576 - DNA, 20 bp, synthetic construct
  source 1..20: mol_type = other DNA
tggatgccatctggacactg

SEQ ID NO: 577 - DNA, 20 bp, synthetic construct
  source 1..20: mol_type = other DNA
tgttcctgggcttcaccaag

SEQ ID NO: 578 - DNA, 20 bp, synthetic construct
  source 1..20: mol_type = other DNA
ttttcaccagcagggcatcg

SEQ ID NO: 579 - DNA, 19 bp, synthetic construct
  source 1..19: mol_type = other DNA
cagctgctgcagtgtttcg

SEQ ID NO: 580 - DNA, 22 bp, synthetic construct
  source 1..22: mol_type = other DNA
agctcgtaggagttcatggttc

SEQ ID NO: 581 - DNA, 19 bp, synthetic construct
  source 1..19: mol_type = other DNA
cctctgtgacgagctcacg

SEQ ID NO: 582 - DNA, 22 bp, synthetic construct
  source 1..22: mol_type = other DNA
ctgagcagttacactcctcaca

SEQ ID NO: 583 - DNA, 20 bp, synthetic construct
  source 1..20: mol_type = other DNA
gctatgccctagtggtggag

SEQ ID NO: 584 - DNA, 19 bp, synthetic construct
  source 1..19: mol_type = other DNA
gcacagggtgctgtacagg

SEQ ID NO: 585 - DNA, 19 bp, synthetic construct
  source 1..19: mol_type = other DNA
ctaccagccctggcagttc

SEQ ID NO: 586 - DNA, 18 bp, synthetic construct
  source 1..18: mol_type = other DNA
gggcacgatgcgtgagta

SEQ ID NO: 587 - DNA, 19 bp, synthetic construct
  source 1..19: mol_type = other DNA
gctgcagcacagagattgc

SEQ ID NO: 588 - DNA, 20 bp, synthetic construct
  source 1..20: mol_type = other DNA
ttcaaagcctctcgcaggtc

SEQ ID NO: 589 - DNA, 22 bp, synthetic construct
  source 1..22: mol_type = other DNA
cggctactttcacttccctctc

SEQ ID NO: 590 - DNA, 18 bp, synthetic construct
  source 1..18: mol_type = other DNA
cggcaccggtcacaatga

SEQ ID NO: 591 - DNA, 19 bp, synthetic construct
  source 1..19: mol_type = other DNA
cactgctctgctgaaggct

SEQ ID NO: 592 - DNA, 19 bp, synthetic construct
  source 1..19: mol_type = other DNA
gagccagcttcgcagtagg

SEQ ID NO: 593 - DNA, 19 bp, synthetic construct
  source 1..19: mol_type = other DNA
ctgcgagggaccactacct

SEQ ID NO: 594 - DNA, 20 bp, synthetic construct
  source 1..20: mol_type = other DNA
ccctccagctgaagttctcg

SEQ ID NO: 595 - DNA, 19 bp, synthetic construct
  source 1..19: mol_type = other DNA
cctggagcagctgcagatc

SEQ ID NO: 596 - DNA, 20 bp, synthetic construct
  source 1..20: mol_type = other DNA
cacaggcacagctccacatt

SEQ ID NO: 597 - DNA, 20 bp, synthetic construct
  source 1..20: mol_type = other DNA
gcatcaatgccagctccatg

SEQ ID NO: 598 - DNA, 18 bp, synthetic construct
  source 1..18: mol_type = other DNA
tctgctgctccagcacct

SEQ ID NO: 599 - DNA, 20 bp, synthetic construct
  source 1..20: mol_type = other DNA
gggatacccaggaccacctg

SEQ ID NO: 600 - DNA, 20 bp, synthetic construct
  source 1..20: mol_type = other DNA
tccgggctgaactcctcaat

SEQ ID NO: 601 - DNA, 22 bp, synthetic construct
  source 1..22: mol_type = other DNA
ggagcaggacaatgatctggag

SEQ ID NO: 602 - DNA, 19 bp, synthetic construct
  source 1..19: mol_type = other DNA
ccagggccttgatgccttt

SEQ ID NO: 603 - DNA, 20 bp, synthetic construct
  source 1..20: mol_type = other DNA
tcgtcctccaatgacaccag

SEQ ID NO: 604 - DNA, 21 bp, synthetic construct
  source 1..21: mol_type = other DNA
gaagttgggtttgcacagaca

SEQ ID NO: 605 - DNA, 18 bp, synthetic construct
  source 1..18: mol_type = other DNA
caactgcacccggtgcga

SEQ ID NO: 606 - DNA, 22 bp, synthetic construct
  source 1..22: mol_type = other DNA
cgcagccatcgaaaccaaaatg

SEQ ID NO: 607 - DNA, 20 bp, synthetic construct
  source 1..20: mol_type = other DNA
atcatcctcagggactgcca

SEQ ID NO: 608 - DNA, 19 bp, synthetic construct
  source 1..19: mol_type = other DNA
gctctgcatcagggtccac

SEQ ID NO: 609 - DNA, 19 bp, synthetic construct
  source 1..19: mol_type = other DNA
ctggcgtctgtgtggactg

SEQ ID NO: 610 - DNA, 20 bp, synthetic construct
  source 1..20: mol_type = other DNA
tgttggaaggcactgagagg

SEQ ID NO: 611 - DNA, 20 bp, synthetic construct
  source 1..20: mol_type = other DNA
cattcctggagccggcatac

SEQ ID NO: 612 - DNA, 19 bp, synthetic construct
  source 1..19: mol_type = other DNA
tggccagcaccatcatgag

SEQ ID NO: 613 - DNA, 20 bp, synthetic construct
  source 1..20: mol_type = other DNA
agagcctggatggcacctat

SEQ ID NO: 614 - DNA, 19 bp, synthetic construct
  source 1..19: mol_type = other DNA
cgagtcgagagggtggttg

SEQ ID NO: 615 - DNA, 18 bp, synthetic construct
  source 1..18: mol_type = other DNA
gggtctctgtgcgagagg

SEQ ID NO: 616 - DNA, 20 bp, synthetic construct
  source 1..20: mol_type = other DNA
gcacaaagggctctccgaag

SEQ ID NO: 617 - DNA, 22 bp, synthetic construct
  source 1..22: mol_type = other DNA
ggacctttactgcaagctggta

SEQ ID NO: 618 - DNA, 19 bp, synthetic construct
  source 1..19: mol_type = other DNA
cgtgccatcgatggcattg

SEQ ID NO: 619 - DNA, 18 bp, synthetic construct
  source 1..18: mol_type = other DNA
gggctacgcgcagatggc

SEQ ID NO: 620 - DNA, 19 bp, synthetic construct
  source 1..19: mol_type = other DNA
gacccgcccgctcacactc

SEQ ID NO: 621 - DNA, 22 bp, synthetic construct
  source 1..22: mol_type = other DNA
cacctctgaggtatttgtgcag

SEQ ID NO: 622 - DNA, 26 bp, synthetic construct
  source 1..26: mol_type = other DNA
agattcagcagaaaattagacgttct

SEQ ID NO: 623 - DNA, 21 bp, synthetic construct
  source 1..21: mol_type = other DNA
aatacaaccatcaccggcttt

SEQ ID NO: 624 - DNA, 23 bp, synthetic construct
  source 1..23: mol_type = other DNA
ccatccgaatgaatttgcatgct

SEQ ID NO: 625 - DNA, 22 bp, synthetic construct
  source 1..22: mol_type = other DNA
ccaccatgactcgttacttctg

SEQ ID NO: 626 - DNA, 20 bp, synthetic construct
  source 1..20: mol_type = other DNA
tccacagccatagttcaggg

SEQ ID NO: 627 - DNA, 19 bp, synthetic construct
  source 1..19: mol_type = other DNA
ggctgtggatgcaatggct

SEQ ID NO: 628 - DNA, 20 bp, synthetic construct
  source 1..20: mol_type = other DNA
tcagtaggtgctgtagccaa

SEQ ID NO: 629 - DNA, 18 bp, synthetic construct
  source 1..18: mol_type = other DNA
cagctgctcctcgggctg

SEQ ID NO: 630 - DNA, 19 bp, synthetic construct
  source 1..19: mol_type = other DNA
cttgaagctcacgggcaca

SEQ ID NO: 631 - DNA, 18 bp, synthetic construct
  source 1..18: mol_type = other DNA
tgtgagctgcaggcctgt

SEQ ID NO: 632 - DNA, 20 bp, synthetic construct
  source 1..20: mol_type = other DNA
agcgggtctggagattcatg

SEQ ID NO: 633 - DNA, 18 bp, synthetic construct
  source 1..18: mol_type = other DNA
acacgccaccatgtgcca

SEQ ID NO: 634 - DNA, 18 bp, synthetic construct
  source 1..18: mol_type = other DNA
cacggctggcttgaagct

SEQ ID NO: 635 - DNA, 19 bp, synthetic construct
  source 1..19: mol_type = other DNA
gctgctcccagtgcagatg

SEQ ID NO: 636 - DNA, 30 bp, synthetic construct
  source 1..30: mol_type = other DNA
ccagatcattctattatattctcgatccag

SEQ ID NO: 637 - DNA, 21 bp, synthetic construct
  source 1..21: mol_type = other DNA
ggattcctcttcatgctgcca

SEQ ID NO: 638 - DNA, 18 bp, synthetic construct
  source 1..18: mol_type = other DNA
caagccggctggcagcta

SEQ ID NO: 639 - DNA, 19 bp, synthetic construct
  source 1..19: mol_type = other DNA
gctcctgcacaccctcatg

SEQ ID NO: 640 - DNA, 21 bp, synthetic construct
  source 1..21: mol_type = other DNA
gaggaatcctcagagcaggtg

SEQ ID NO: 641 - DNA, 18 bp, synthetic construct
  source 1..18: mol_type = other DNA
tgccagctggcttgctgt

SEQ ID NO: 642 - DNA, 20 bp, synthetic construct
  source 1..20: mol_type = other DNA
actgctggcagcatgaagaa

SEQ ID NO: 643 - DNA, 20 bp, synthetic construct
  source 1..20: mol_type = other DNA
tgactcttgctccgactcct

SEQ ID NO: 644 - DNA, 18 bp, synthetic construct
  source 1..18: mol_type = other DNA
caggtcactgggcagcag

SEQ ID NO: 645 - DNA, 26 bp, synthetic construct
  source 1..26: mol_type = other DNA
gctagtagcaactgatgtttcttagt

SEQ ID NO: 646 - DNA, 24 bp, synthetic construct
  source 1..24: mol_type = other DNA
ccagagcaacattgaatagcattg

SEQ ID NO: 647 - DNA, 26 bp, synthetic construct
  source 1..26: mol_type = other DNA
agcaccaccttctagggataaataaa

SEQ ID NO: 648 - DNA, 21 bp, synthetic construct
  source 1..21: mol_type = other DNA
agtttcccgagcagtagttgg

SEQ ID NO: 649 - DNA, 22 bp, synthetic construct
  source 1..22: mol_type = other DNA
gggaaactccaactcaggatct

SEQ ID NO: 650 - DNA, 20 bp, synthetic construct
  source 1..20: mol_type = other DNA
gtctgtgacccaggtatggt

SEQ ID NO: 651 - DNA, 26 bp, synthetic construct
  source 1..26: mol_type = other DNA
ctattaaaactcagtcgcattggttt

SEQ ID NO: 652 - DNA, 25 bp, synthetic construct
  source 1..25: mol_type = other DNA
tgtatgaagatgcatcagaactctt

(19) United States
(12) Patent Application Publication
(10) Pub. No.: US 2026/0117303 A1
(43) Pub. Date: Apr. 30, 2026

(54) PRIMER SET AND KIT FOR INDIVIDUAL IDENTIFICATION OF HAIR SHAFT, AND USE THEREOF

(71) Applicant: Shanxi Medical University, Taiyuan City (CN)

(72) Inventors: Gengqian ZHANG, Taiyuan City (CN); Jinding LIU, Taiyuan City (CN); Jiangwei YAN, Taiyuan City (CN); Keming YUN, Taiyuan City (CN); Yuxin ZHANG, Taiyuan City (CN)

(73) Assignee: Shanxi Medical University, Taiyuan City (CN)

(21) Appl. No.: 19/374,830

(22) Filed: Oct. 30, 2025

(30) Foreign Application Priority Data
Oct. 31, 2024 (CN) 202411533695.X

(51) Int. Cl. C12Q 1/6881

(52) U.S. Cl. CPC C12Q 1/6881; C12Q 2600/156

(57) ABSTRACT

A primer set and a kit for individual identification of a hair shaft, and use thereof, which belongs to the technical field of forensic science. The primer set enables the detection of coding single nucleotide polymorphism (cSNP) sites and/or microhaplotype (MH) sites on highly-expressed mRNA markers in hair shafts, addressing the challenge of individual identification using hair shaft samples with high accuracy. Additionally, instruments and reagents required are conventionally available in forensic laboratories, making the detection straightforward to conduct and easy to promote.

### CROSS REFERENCE TO RELATED APPLICATION

This patent application claims the benefit and priority of Chinese Patent Application No. 202411533695.X filed with the China National Intellectual Property Administration on Oct. 31, 2024, the disclosure of which is incorporated by reference herein in its entirety as part of the present application.

### REFERENCE TO SEQUENCE LISTING

A computer readable XML file entitled “GWP20250902157”, that was created on Oct. 17, 2025, with a file size of about 576,035 bytes, contains the sequence listing for this application, has been filed with this application, and is hereby incorporated by reference in its entirety.

### TECHNICAL FIELD

The present disclosure belongs to the technical field of forensic science, and specifically relates to a primer set and a kit for individual identification of a hair shaft, and use thereof.

### BACKGROUND

Hair samples are one of the common types of samples in forensic identification. Forensic investigators generally collect hair samples at crime scenes. However, since hair is mostly naturally shed, the hair follicles are always atrophied or absent, easily leading to detection failure or insufficient nuclear DNA extraction.

Although hair shafts contain a certain amount of DNA, the DNA is extremely low in content and mostly degraded. Nonetheless, some methods for individual identification using hair shafts exist in current research, such as autosomal short tandem repeat (STR) detection, mitochondrial DNA (mtDNA) detection, and single amino-acid polymorphism (SAP) detection. However, due to the micro-degradation nature of hair shaft DNA, autosomal STR detection generally results in the loss of long-fragment loci or allele dropout/insertion in short-fragment loci. Although detecting two hypervariable regions of mtDNA can reveal single nucleotide polymorphism (SNP) differences, mtDNA is maternally inherited and can only be used to exclude individuals of different maternal lineages. While the SAP detection can infer nonsynonymous SNPs in genomic DNA sequences based on changes in SNPs, synonymous mutations in DNA are not reflected in SNPs. This process provides limited information and may lead to incorrect inferences of nonsynonymous SNPs due to chemical or biological modifications of amino acids caused mass shifts during mass spectrometry analysis.

It is evident that existing methods for individual identification using hair shafts all have inherent limitations. There remains a lack of novel genetic markers and detection methods in the field to achieve precise individual identification of hair shaft samples.

### SUMMARY

A purpose of the present disclosure is to provide a primer set and a kit for individual identification of a hair shaft, and use thereof. The primer set and the kit containing the primer set enable the detection of coding single nucleotide polymorphism (cSNP) sites and/or microhaplotype (MH) sites on highly-expressed mRNA markers in hair shafts, addressing the challenge of individual identification using hair shaft samples with high accuracy.

The present disclosure provides use of a reagent for detecting a human gene-derived cSNP site in one or more of mRNA polymorphism detection for individual identification of a hair shaft, forensic identification of a hair shaft specimen, and forensic testing of the hair shaft specimen; where the human gene-derived cSNP site includes 404 cSNPs shown in the following table:

| cSNP | cSNP | cSNP | cSNP | cSNP | cSNP | cSNP | cSNP |
|---|---|---|---|---|---|---|---|
| rs11464 | rs9320182 | rs2257286 | rs931749 | rs611251 | rs3845 | rs2131730 | rs583678 |
| rs1063128 | rs9320183 | rs2248473 | rs73041302 | rs557806 | rs1046373 | rs2131731 | rs372790 |
| rs3820553 | rs9486408 | rs2852468 | rs61160177 | rs610308 | rs142478896 | rs1666573 | rs432498 |
| rs6696123 | rs9486409 | rs2857670 | rs77862444 | rs11541192 | rs143258620 | rs2934418 | rs3741748 |
| rs2273584 | rs2239710 | rs2857671 | rs8106900 | rs556052 | rs145108464 | rs3742966 | rs77813527 |
| rs641615 | rs2071599 | rs2852456 | rs6508786 | rs500079 | rs148576335 | rs937248 | rs1797683 |
| rs641621 | rs1051602 | rs2857672 | rs4386247 | rs524 | rs17152448 | rs3742963 | rs8648 |
| rs1132948 | rs6436712 | rs7968466 | rs118185142 | rs527 | rs3740535 | rs3742964 | rs11755593 |
| rs4761924 | rs112558913 | rs1059837 | rs12609247 | rs7317471 | rs1864253 | rs7166305 | rs2296744 |
| rs11549949 | rs113140754 | rs15667 | rs1142825 | rs558614 | rs150285041 | rs17820431 | rs10947436 |
| rs11549950 | rs113823535 | rs2302613 | rs4242797 | rs2770928 | rs6435927 | rs17820437 | rs2296743 |
| rs6603 | rs486903 | rs2302614 | rs4589188 | rs2304704 | rs1071862 | rs11856 | rs367875497 |
| rs3818831 | rs12896 | rs1049500 | rs4589189 | rs17337252 | rs76239019 | rs8784 | rs9982775 |
| rs2832753 | rs1883 | rs201017624 | rs1131482 | rs2305427 | rs10186663 | rs12439292 | rs9982675 |
| rs2832752 | rs8020483 | rs1876363 | rs4947 | rs1129660 | rs1193 | rs1989365 | rs9982755 |
| rs10437608 | rs7143775 | rs1049205 | rs1059623 | rs7327548 | rs13297 | rs2010201 | rs9982910 |
| rs1554998431 | rs12588499 | rs2075639 | rs10792769 | rs77685055 | rs1049457 | rs12452295 | rs13046903 |
| rs3824886 | rs10289 | rs1049206 | rs2663 | rs1062087 | rs1049458 | rs8072775 | rs7275281 |
| rs3781679 | rs4246 | rs1049207 | rs2664 | rs2297208 | rs11547281 | rs7786826 | , rs7275298 |
| rs16906210 | rs1050784 | rs1049208 | rs2665 | rs557337 | rs3738126 | rs7800753 | rs7276859 |
| rs11205896 | rs2305556 | rs2037912 | rs477859 | rs4881274 | rs818571 | rs2178428 | rs12481809 |
| rs1770791 | rs1047448 | rs1049212 | rs483876 | rs10904083 | rs3750823 | rs3735576 | rs12483730 |
| rs8375 | rs1047449 | rs7214 | rs624313 | rs2288681 | rs10887673 | rs2108192 | rs2838622 |
| rs6503578 | rs3744786 | rs1128416 | rs4925229 | rs3740304 | rs74506939 | rs10269841 | rs7283084 |
| rs9635728 | rs2071561 | rs11687800 | rs11204472 | rs2640738 | rs3826543 | rs3801904 | rs9984476 |
| rs6503577 | rs72830046 | rs7475 | rs8124907 | rs2640785 | rs112542604 | rs2007922 | rs2838621 |
| rs10788862 | rs12948056 | rs3190 | rs2427289 | rs2846412 | rs141856614 | rs3807890 | rs6558394 |
| rs4890107 | rs11078993 | rs2241726 | rs6062223 | rs10749920 | rs3815561 | rs11520636 | rs10866915 |
| rs78283750 | rs2857257 | rs10747559 | rs13038331 | rs2640779 | rs3740497 | rs923935 | rs4965046 |
| rs9863910 | rs2071563 | rs3782357 | rs13042941 | rs3741060 | rs807048 | rs923936 | rs112100339 |
| rs576 | rs2604954 | rs3741622 | rs13044266 | rs4643041 | rs3829598 | rs76443569 | rs12101554 |
| rs704246 | rs2604955 | rs11168830 | rs6142734 | rs12233086 | rs9890989 | rs936524 | rs2130882 |
| rs4946758 | rs2604953 | rs7908387 | rs2151513 | rs7558074 | rs9897046 | rs12983010 | rs3803385 |
| rs1340623 | rs71373411 | rs1681 | rs3810548 | rs11896520 | rs3813048 | rs73038948 | rs3169159 |
| rs4945755 | rs743686 | rs2076190 | rs7262393 | rs1376443 | rs3813050 | rs4801861 | rs3169160 |
| rs4946759 | rs7216017 | rs2231301 | rs2427285 | rs2290257 | rs3813047 | rs1060186 | rs9652498 |
| rs11152999 | rs12451652 | rs1950252 | rs2427284 | rs9308819 | rs375280428 | rs201833096 | rs1043372 |
| rs1159148 | rs2071601 | rs10149339 | rs11698080 | rs2290258 | rs147906218 | rs2885799 | rs1062702 |
| rs3747787 | rs2239711 | rs12937519 | rs2427283 | rs77272400 | rs76327324 | rs411107 | rs1043374 |
| rs2297970 | rs79897879 | rs16966703 | rs3737137 | rs1129235 | rs80168553 | rs419374 | rs1043382 |
| rs3747790 | rs2071588 | rs2076304 | rs6143021 | rs8121 | rs72625995 | rs556681459 | rs201732843 |
| rs11537704 | rs3187034 | rs2064217 | rs2297587 | rs14039 | rs77163010 | rs11608915 | rs233309 |
| rs1057429 | rs7300801 | rs2076299 | rs6089732 | rs3173082 | rs7212517 | rs382367 | rs13051603 |
| rs1135384 | rs4761784 | rs6929069 | rs944895 | rs3777668 | rs72625994 | rs3894847 | rs58757665 |
| rs6922741 | rs4761856 | rs2076300 | rs4755571 | rs7745062 | rs6503593 | rs412533 | rs9980630 |
| rs9486407 | rs3657 | rs2744380 | rs10600 | rs7745259 | rs8067655 | rs410562 | rs233303 |
| rs13319 | rs2857663 | rs12250 | rs2074478 | rs3777667 | rs7182648 | rs570463693 | rs78731676 |
| rs1057433 | rs3741715 | rs3745951 | rs1265099 | rs8476 | rs1280396 | rs1053796 | rs3804008 |
| rs8013 | rs2857667 | rs34989303 | rs3132558 | rs17152433 | rs1620402 | rs2567 | rs3804007 |
| rs11758586 | rs2852464 | rs3745952 | rs626974 | rs3906796 | rs16977594 | rs2568 | rs2236427 |
| rs6933677 | rs2857669 | rs931750 | rs564196 | — | — | — | — |

The present disclosure further provides a primer set for individual identification of a hair shaft, including 326 primer pairs, where the 326 primer pairs have nucleotide sequences set forth in SEQ ID NO: 1 to SEQ ID NO: 652.

The present disclosure further provides a kit for individual identification of a hair shaft, including the primer set.

The present disclosure further provides use of the primer set or the kit in one or more of mRNA polymorphism detection for individual identification of a hair shaft, forensic identification of a hair shaft specimen, and forensic testing of the hair shaft specimen.

The present disclosure further provides a method for detecting mRNA polymorphism for individual identification of a hair shaft, including the following steps:
extracting RNA from a hair shaft sample to be tested, and then subjecting the RNA from the hair shaft sample to be tested to reverse transcription, thereby obtaining a hair shaft sample-derived cDNA;
subjecting the hair shaft sample-derived cDNA to multiplex PCR amplification using the primer set to obtain a hair shaft sample-derived multiplex PCR amplification product;
subjecting the hair shaft sample-derived multiplex PCR amplification product to library construction, thereby obtaining a hair shaft sample-derived sequencing library;
subjecting the hair shaft sample-derived sequencing library to high-throughput sequencing, thereby obtaining a high-throughput sequencing result; and
subjecting the high-throughput sequencing result to genotyping, thereby obtaining a cSNP genotyping result and/or a microhaplotype (MH) genotyping result.

### Beneficial Effects

The present disclosure provides use of a reagent for detecting a human gene-derived cSNP site in one or more of mRNA polymorphism detection for individual identification of a hair shaft, forensic identification of a hair shaft specimen, and forensic testing of the hair shaft specimen; where the human gene-derived cSNP site includes 404 cSNPs. Furthermore, the present disclosure further provides a primer set capable of amplifying the 404 cSNPs, a kit, and use thereof using RNA as a template in mRNA polymorphism detection for individual identification of a hair shaft, forensic identification of a hair shaft specimen, and forensic testing of the hair shaft specimen.

Compared to methods that use hair shaft DNA as a template for individual detection in prior art, the present disclosure employs RNA as the template for detection. This approach ensures that even if the hair shaft is a micro-degraded sample, RNA, being present in multiple copies, exists in sufficient quantities within the hair shaft. This provides an adequate template for subsequent detection, thereby overcoming the challenge of insufficient nuclear DNA extraction from hair shafts. Additionally, RNA molecules contain polymorphic sites suitable for individual identification, namely the 404 cSNPs. As the polymorphism of the DNA sequence is retained in the RNA sequence, RNA polymorphism can reflect DNA polymorphism. Moreover, the primer set enables the detection of cSNP sites and/or MH sites on highly-expressed mRNA markers in hair shafts, addressing the challenge of individual identification using hair shaft samples with high accuracy. Additionally, instruments and reagents required are conventionally available in forensic laboratories, making the detection straightforward to conduct and easy to promote.

### DETAILED DESCRIPTION OF THE EMBODIMENTS

The present disclosure provides use of a reagent for detecting a human gene-derived cSNP site in one or more of mRNA polymorphism detection for individual identification of a hair shaft, forensic identification of a hair shaft specimen, and forensic testing of the hair shaft specimen; where the human gene-derived cSNP site includes 404 cSNPs. The table containing the 404 cSNPs has been provided in the “SUMMARY” section and will not be repeated here.

In one embodiment of the present disclosure, the reagent includes a primer set for amplifying the 404 cSNPs; in another embodiment, the primer set includes 326 primer pairs, and the 326 primer pairs have nucleotide sequences set forth in SEQ ID NO: 1 to SEQ ID NO: 652.

The present disclosure further provides a primer set for individual identification of a hair shaft, including 326 primer pairs, where the 326 primer pairs have nucleotide sequences set forth in SEQ ID NO: 1 to SEQ ID NO: 652.

The present disclosure further provides a kit for individual identification of a hair shaft, including the primer set. In one embodiment of the present disclosure, the kit may further include an RNA extraction-related reagent and/or an RNA reverse transcription-related reagent; in another embodiment, the kit may further include the RNA extraction-related reagent and the RNA reverse transcription-related reagent; in another embodiment, the kit may further include a high-throughput library construction-related reagent and/or a high-throughput sequencing-related reagent; in another embodiment, the kit may further include the high-throughput library construction-related reagent and the high-throughput sequencing-related reagent. There are no specific limitations regarding the source and specifications of the RNA extraction-related reagent, RNA reverse transcription-related reagent, and high-throughput library construction and high-throughput sequencing-related reagents, and commercially available reagents in the field may be used.

The present disclosure further provides use of the primer set or the kit in one or more of mRNA polymorphism detection for individual identification of a hair shaft, forensic identification of a hair shaft specimen, and forensic testing of the hair shaft specimen.

Specifically, the present disclosure further provides a method for detecting mRNA polymorphism for individual identification of a hair shaft, including the following steps:
extracting RNA from a hair shaft sample to be tested, and then subjecting the RNA from the hair shaft sample to be tested to reverse transcription, thereby obtaining a hair shaft sample-derived cDNA;
subjecting the hair shaft sample-derived cDNA to multiplex PCR amplification using the primer set to obtain a hair shaft sample-derived multiplex PCR amplification product;
subjecting the hair shaft sample-derived multiplex PCR amplification product to library construction, thereby obtaining a hair shaft sample-derived sequencing library;
subjecting the hair shaft sample-derived sequencing library to high-throughput sequencing, thereby obtaining a high-throughput sequencing result; and
subjecting the high-throughput sequencing result to genotyping, thereby obtaining a cSNP genotyping result and/or a microhaplotype (MH) genotyping result.

In one embodiment of the present disclosure, the genotyping may refer to data analysis processes documented in literature (Liu Z, Wang J, Li L, Yang H, Yu H, Fan J, Zhang M, Zhang Y, Liu J, Li Z, Zhang G. Identification of the body fluid donor in mixtures through target mRNA cSNP sequencing. Forensic Sci Int Genet. 2024 July; 71:103066. doi: 10.1016/j.fsigen. 2024.103066. Epub 2024 May 31. PMID: 38833776), using the transcript data of genes where the 404 cSNPs are located as a reference sequence. Information related to these genes and their transcripts is detailed in Table 1. In another embodiment, a “reads” count threshold is set to 30 for amplicons during the genotyping, instead of the conventional threshold of 200 to 500, to accommodate the nature of hair shaft samples as micro-degraded samples.

In the present disclosure, there are no specific limitations regarding the processes of RNA extraction, RNA reverse transcription, multiplex PCR amplification, and library construction, and they may be conducted according to procedures and methods well-known to those skilled in the art. There are no specific requirements for a high-throughput sequencing platform; conventional high-throughput sequencing platforms in the field may be selected based on the adapters used for library construction. For example, in specific examples, the BGI DNBSEQ™ platform is adopted.

In order to further illustrate the present disclosure, the technical solutions provided by the present disclosure are described in detail below in connection with examples, but these examples should not be understood as limiting the claimed scope of the present disclosure.

### Example 1

An mRNA polymorphism detection site for individual identification of hair shaft samples and its primer sequences consisted of the following sites and primers: the sites were shown in Table 1 (the rs numbers of the cSNPs in Table 1 were the accession numbers in the Ensembl database and NCBI database), and the primers were shown in Table 2.

TABLE 1
Information on screened genes and cSNPs

| Gene | Transcript number | NM number | Chromosome | Position | cSNP | Alleles |
|---|---|---|---|---|---|---|
| LYST-201 | ENST00000389793.7 | NM_000081.4 | 1 | 235830265 | rs11464 | T/G |
| | | | 1 | 235809567 | rs1063128 | T/C |
| | | | 1 | 235809135 | rs3820553 | T/C |
| | | | 1 | 235781994 | rs6696123 | T/C |
| | | | 1 | 235777150 | rs2273584 | C/T |
| KRT5A-201 | ENST00000252242.9 | NM_000424.4 | 12 | 52519125 | rs641615 | G/C |
| | | | 12 | 52519122 | rs641621 | G/A |
| | | | 12 | 52518984 | rs1132948 | C/G |
| | | | 12 | 52517617 | rs4761924 | T/G |
| | | | 12 | 52515088 | rs11549949 | C/A |
| | | | 12 | 52515133 | rs11549950 | T/C |
| | | | 12 | 52514689 | rs6603 | G/A |
| FLG2-201 | ENST00000388718.5 | NM_001014342.3 | 1 | 152358764 | rs3818831 | G/A |
| KRTAP24-1-201 | ENST00000340345.6 | NM_001085455.3 | 21 | 30282130 | rs2832753 | T/C |
| | | | 21 | 30281816 | rs2832752 | G/C |
| HTATIP2-204 | ENST00000451739.7 | NM_001098522.2 | 11 | 20364060 | rs10437608 | G/A |
| | | | 11 | 20364075 | rs1554998431 | G/C |
| | | | 11 | 20383067 | rs3824886 | T/G |
| | | | 11 | 20383211 | rs3781679 | T/A |
| | | | 11 | 20383589 | rs16906210 | C/A |
| NRDC-201 | ENST00000352171.12 | NM_001101662.2 | 1 | 51825312 | rs11205896 | T/A |
| | | | 1 | 51798392 | rs1770791 | A/G |
| | | | 1 | 51800570 | rs8375 | G/A |
| KRTAP2-1-201 | ENST00000391419.3 | NM_001123387.1 | 17 | 41047238 | rs6503578 | G/A |
| | | | 17 | 41047203 | rs9635728 | G/A |
| | | | 17 | 41046675 | rs6503577 | T/A |
| PRR9-201 | ENST00000368744.4 | NM_001195571.2 | 1 | 153219036 | rs10788862 | T/A |
| KRTAP9-7-201 | ENST00000391354.2 | NM_001277332.1 | 17 | 41275765 | rs4890107 | C/G |
| | | | 17 | 41275755 | rs78283750 | A/C |
| MITF-203 | ENST00000352241.9 | NM_001354604.2 | 3 | 69965296 | rs9863910 | T/C |
| | | | 3 | 69967316 | rs576 | C/G |
| | | | 3 | 69967753 | rs704246 | C/A |
| CRYBG1-203 | ENST00000633556.3 | NM_001371242.2 | 6 | 106511549 | rs4946758 | A/G |
| | | | 6 | 106512124 | rs1340623 | C/A |
| | | | 6 | 106512164 | rs4945755 | G/A |
| | | | 6 | 106512086 | rs4946759 | A/C |
| | | | 6 | 106512572 | rs11152999 | G/A |
| | | | 6 | 106369254 | rs1159148 | A/C |
| | | | 6 | 106519903 | rs3747787 | T/C |
| | | | 6 | 106551947 | rs2297970 | G/A |
| | | | 6 | 106568856 | rs3747790 | T/A |
| | | | 6 | 106569067 | rs11537704 | C/T |
| | | | 6 | 106523879 | rs1057429 | G/A |
| | | | 6 | 106569395 | rs1135384 | T/C |
| | | | 6 | 106569424 | rs6922741 | C/T |
| | | | 6 | 106569443 | rs9486407 | G/A |
| | | | 6 | 106569779 | rs13319 | G/A |
| | | | 6 | 106570051 | rs1057433 | C/A |
| | | | 6 | 106570279 | rs8013 | A/C |
| | | | 6 | 106570571 | rs11758586 | T/C |
| | | | 6 | 106570799 | rs6933677 | A/G |
| | | | 6 | 106571412 | rs9320182 | C/T |
| | | | 6 | 106571760 | rs9320183 | A/G |
| | | | 6 | 106571786 | rs9486408 | T/C |
| | | | 6 | 106571791 | rs9486409 | A/G |
| KRT34-201 | ENST00000394001.3 | NM_001386014.1 | 17 | 41379607 | rs2239710 | A/C |
| | | | 17 | 41379136 | rs2071599 | T/C |
| | | | 17 | 41377784 | rs1051602 | T/A |
| SCYGR3-201 | ENST00000642029.1 | NM_001395404.1 | 2 | 227614658 | rs6436712 | T/A |
| SCYGR4-201 | ENST00000641801.2 | NM_001395405.1 | 2 | 227617594 | rs112558913 | G/A |
| | | | 2 | 227617605 | rs113140754 | T/C |
| SCYGR6-201 | ENST00000641918.1 | NM_001395407.1 | 2 | 227724523 | rs113823535 | C/T |
| AHNAK-202 | ENST00000378024.9 | NM_001620.3 | 11 | 62526476 | rs486903 | G/A |
| EIF5-201 | ENST00000216554.8 | NM_001969.5 | 14 | 103334467 | rs12896 | C/T |
| | | | 14 | 103341816 | rs1883 | A/G |
| | | | 14 | 103342135 | rs8020483 | C/T |
| | | | 14 | 103343504 | rs7143775 | A/C |
| | | | 14 | 103343840 | rs12588499 | T/A |
| | | | 14 | 103344576 | rs10289 | A/G |
| | | | 14 | 103344912 | rs4246 | A/C |
| KRT15-201 | ENST00000254043.8 | NM_002275.4 | 17 | 41518389 | rs1050784 | T/A |
| | | | 17 | 41515472 | rs2305556 | T/C |
| KRT31-201 | ENST00000251645.3 | NM_002277.3 | 17 | 41394000 | rs1047448 | T/C |
| | | | 17 | 41393899 | rs1047449 | G/A |
| KRT32-201 | ENST00000225899.4 | NM_002278.3 | 17 | 41467111 | rs3744786 | T/C |
| | | | 17 | 41465816 | rs2071561 | G/T |
| | | | 17 | 41464313 | rs72830046 | C/T |
| | | | 17 | 41464147 | rs12948056 | C/A |
| | | | 17 | 41462941 | rs11078993 | C/T |
| | | | 17 | 41462967 | rs2857257 | G/A |
| | | | 17 | 41462863 | rs2071563 | G/A |
| | | | 17 | 41462841 | rs2604954 | G/A |
| | | | 17 | 41462842 | rs2604955 | T/C |
| | | | 17 | 41460178 | rs2604953 | G/A |
| KRT33B-201 | ENST00000251646.8 | NM_002279.5 | 17 | 41364996 | rs71373411 | A/C |
| KRT35-201 | ENST00000246639.7 | NM_002280.6 | 17 | 41480992 | rs743686 | A/G |
| | | | 17 | 41478373 | rs7216017 | A/G |
| | | | 17 | 41477102 | rs12451652 | C/T |
| | | | 17 | 41477097 | rs2071601 | G/C |
| | | | 17 | 41477065 | rs2239711 | A/C |
| KRT81-201 | ENST00000327741.9 | NM_002281.4 | 12 | 52291429 | rs79897879 | T/G |
| | | | 12 | 52291312 | rs2071588 | C/A |
| | | | 12 | 52287653 | rs3187034 | A/C |
| | | | 12 | 52287632 | rs7300801 | T/C |
| | | | 12 | 52287296 | rs4761784 | G/A |
| | | | 12 | 52287272 | rs4761856 | C/A |
| | | | 12 | 52286224 | rs3657 | T/A |
| KRT83-201 | ENST00000293670.3 | NM_002282.3 | 12 | 52319304 | rs2857663 | G/A |
| | | | 12 | 52319191 | rs3741715 | G/A |
| | | | 12 | 52317765 | rs2857667 | G/A |
| | | | 12 | 52316937 | rs2852464 | G/A |
| | | | 12 | 52316931 | rs2857669 | G/A |
| | | | 12 | 52316525 | rs2257286 | G/A |
| | | | 12 | 52316099 | rs2248473 | T/C |
| | | | 12 | 52316096 | rs2852468 | A/G |
| | | | 12 | 52314781 | rs2857670 | A/C |
| | | | 12 | 52314636 | rs2857671 | G/A |
| | | | 12 | 52314457 | rs2852456 | G/A |
| | | | 12 | 52314493 | rs2857672 | G/A |
| KRT85-201 | ENST00000257901.7 | NM_002283.4 | 12 | 52360731 | rs7968466 | T/A |
| | | | 12 | 52360551 | rs1059837 | G/A |
| | | | 12 | 52360077 | rs15667 | C/T |
| ODC1-201 | ENST00000234111.9 | NM_002539.3 | 2 | 10445176 | rs2302613 | T/C |
| | | | 2 | 10445185 | rs2302614 | A/C |
| | | | 2 | 10440841 | rs1049500 | G/A |
| PABPC1-201 | ENST00000318607.10 | NM_002568.4 | 8 | 100709477 | rs201017624 | G/T |
| PPL-201 | ENST00000345988.7 | NM_002705.5 | 16 | 4894517 | rs1876363 | C/G |
| | | | 16 | 4892098 | rs1049205 | C/T |
| | | | 16 | 4885635 | rs2075639 | G/A |
| | | | 16 | 4885145 | rs1049206 | C/A |
| | | | 16 | 4884563 | rs1049207 | C/G |
| | | | 16 | 4884548 | rs1049208 | G/A |
| | | | 16 | 4883938 | rs2037912 | G/A |
| | | | 16 | 4882928 | rs1049212 | A/C |
| | | | 16 | 4882559 | rs7214 | T/C |
| PPP1CB-202 | ENST00000395366.3 | NM_002709.3 | 2 | 28778825 | rs1128416 | A/C |
| | | | 2 | 28800093 | rs11687800 | T/C |
| | | | 2 | 28800883 | rs7475 | A/G |
| | | | 2 | 28802613 | rs3190 | A/C |
| KMT2D-201 | ENST00000301067.12 | NM_003482.4 | 12 | 49050762 | rs2241726 | G/A |
| | | | 12 | 49040291 | rs10747559 | C/A |
| | | | 12 | 49033869 | rs3782357 | C/A |
| | | | 12 | 49032195 | rs3741622 | T/A |
| | | | 12 | 49030751 | rs11168830 | G/A |
| EIF3A-201 | ENST00000369144.8 | NM_003750.4 | 10 | 119069457 | rs7908387 | A/C |
| MAP7-201 | ENST00000354570.8 | NM_003980.6 | 6 | 136342810 | rs1681 | A/C |
| | | | 6 | 136361034 | rs2076190 | G/A |
| BCL2L2-201 | ENST00000250405.10 | NM_004050.5 | 14 | 23307890 | rs2231301 | G/A |
| | | | 14 | 23309489 | rs1950252 | A/C |
| | | | 14 | 23311013 | rs10149339 | A/G |
| KRT33A-201 | ENST00000007735.4 | NM_004138.4 | 17 | 41346911 | rs12937519 | G/A |
| | | | 17 | 41346146 | rs16966703 | A/G |
| DSP-201 | ENST00000379802.8 | NM_004415.4 | 6 | 7572029 | rs2076304 | A/C |
| | | | 6 | 7577027 | rs2064217 | C/T |
| | | | 6 | 7580725 | rs2076299 | A/G |
| | | | 6 | 7581403 | rs6929069 | G/A |
| | | | 6 | 7584384 | rs2076300 | C/T |
| | | | 6 | 7585734 | rs2744380 | G/A |
| | | | 6 | 7586075 | rs12250 | C/T |
| KCNK6-201 | ENST00000263372.5 | NM_004823.3 | 19 | 38326846 | rs3745951 | T/C |
| | | | 19 | 38327236 | rs34989303 | G/A |
| | | | 19 | 38327659 | rs3745952 | C/T |
| | | | 19 | 38328111 | rs931750 | T/C |
| | | | 19 | 38328285 | rs931749 | C/T |
| | | | 19 | 38328458 | rs73041302 | A/C |
| | | | 19 | 38328823 | rs61160177 | C/A |
| | | | 19 | 38329888 | rs77862444 | C/A |
| | | | 19 | 38330401 | rs8106900 | G/T |
| | | | 19 | 38330642 | rs6508786 | G/A |
| | | | 19 | 38330834 | rs4386247 | T/C |
| | | | 19 | 38331069 | rs118185142 | G/A |
| | | | 19 | 38331057 | rs12609247 | C/T |
| CALML3-201 | ENST00000315238.3 | NM_005185.4 | 10 | 5525403 | rs1142825 | G/A |
| | | | 10 | 5525732 | rs4242797 | G/A |
| | | | 10 | 5525951 | rs4589188 | G/A |
| | | | 10 | 5525901 | rs4589189 | G/C |
| | | | 10 | 5526004 | rs1131482 | G/A |
| HSP90AA1-201 | ENST00000216281.13 | NM_005348.4 | 14 | 102084466 | rs4947 | G/A |
| | | | 14 | 102080881 | rs1059623 | G/A |
| KRTAP5-9-201 | ENST00000528743.4 | NM_005553.4 | 11 | 71548776 | rs10792769 | A/G |
| | | | 11 | 71549250 | rs2663 | G/A |
| | | | 11 | 71549197 | rs2664 | T/A |
| | | | 11 | 71549302 | rs2665 | T/A |
| LAMA5-201 | ENST00000252999.7 | NM_005560.6 | 20 | 62362541 | rs477859 | A/G |
| | | | 20 | 62352293 | rs483876 | C/T |
| | | | 20 | 62352356 | rs624313 | G/A |
| | | | 20 | 62346587 | rs4925229 | T/C |
| | | | 20 | 62345831 | rs11204472 | G/A |
| | | | 20 | 62338071 | rs8124907 | A/G |
| | | | 20 | 62337627 | rs2427289 | T/A |
| | | | 20 | 62334260 | rs6062223 | C/T |
| | | | 20 | 62334004 | rs13038331 | A/C |
| | | | 20 | 62333913 | rs13042941 | T/C |
| | | | 20 | 62333908 | rs13044266 | G/A |
| | | | 20 | 62332619 | rs6142734 | G/A |
| | | | 20 | 62332390 | rs2151513 | T/A |
| | | | 20 | 62330822 | rs3810548 | A/C |
| | | | 20 | 62328363 | rs7262393 | C/G |
| | | | 20 | 62325523 | rs2427285 | C/A |
| | | | 20 | 62325425 | rs2427284 | A/C |
| | | | 20 | 62324126 | rs11698080 | C/G |
| | | | 20 | 62324150 | rs2427283 | C/G |
| | | | 20 | 62322665 | rs3737137 | C/T |
| | | | 20 | 62322716 | rs6143021 | T/C |
| | | | 20 | 62320641 | rs2297587 | C/T |
| | | | 20 | 62318583 | rs6089732 | G/A |
| | | | 20 | 62312525 | rs944895 | G/A |
| HIPK3-201 | ENST00000303296.9 | NM_005734.5 | 11 | 33354549 | rs4755571 | A/G |
| | | | 11 | 33356663 | rs10600 | A/C |
| PSORS1C2-201 | ENST00000259845.5 | NM_014069.3 | 6 | 31137856 | rs2074478 | C/G |
| | | | 6 | 31137636 | rs1265099 | A/C |
| | | | 6 | 31137689 | rs3132558 | C/A |
| PPP1R15A-201 | ENST00000200453.6 | NM_014330.5 | 19 | 48872643 | rs626974 | T/A |
| | | | 19 | 48873325 | rs564196 | G/A |
| | | | 19 | 48873829 | rs611251 | T/C |
| | | | 19 | 48873985 | rs557806 | G/A |
| | | | 19 | 48874062 | rs610308 | A/G |
| | | | 19 | 48874167 | rs11541192 | G/A |
| | | | 19 | 48874179 | rs556052 | G/C |
| | | | 19 | 48875737 | rs500079 | A/C |
| | | | 19 | 48875910 | rs524 | C/T |
| | | | 19 | 48875925 | rs527 | G/A |
| LATS2-201 | ENST00000382592.5 | NM_014572.3 | 13 | 21045946 | rs7317471 | T/C |
| | | | 13 | 20988809 | rs558614 | G/A |
| | | | 13 | 20988693 | rs2770928 | C/A |
| SLC40A1-201 | ENST00000261024.7 | NM_014585.6 | 2 | 189565451 | rs2304704 | A/G |
| RB1CC1-201 | ENST00000025008.10 | NM_014781.5 | 8 | 52674146 | rs17337252 | A/G |
| | | | 8 | 52642509 | rs2305427 | A/G |
| | | | 8 | 52624756 | rs1129660 | A/C |
| TBC1D4-202 | ENST00000377636.8 | NM_014832.5 | 13 | 75481684 | rs7327548 | G/C |
| | | | 13 | 75481466 | rs77685055 | G/A |
| | | | 13 | 75310080 | rs1062087 | C/T |
| | | | 13 | 75302253 | rs2297208 | G/A |
| | | | 13 | 75286865 | rs557337 | A/G |
| DIP2C-201 | ENST00000280886.12 | NM_014974.3 | 10 | 414000 | rs4881274 | G/A |
| | | | 10 | 384037 | rs10904083 | G/A |
| | | | 10 | 327108 | rs2288681 | T/C |
| | | | 10 | 277343 | rs3740304 | A/G |
| EXPH5-201 | ENST00000265843.9 | NM_015065.3 | 11 | 108593482 | rs2640738 | T/C |
| | | | 11 | 108539057 | rs2640785 | T/A |
| | | | 11 | 108513480 | rs2846412 | C/G |
| | | | 11 | 108512949 | rs10749920 | A/G |
| | | | 11 | 108510520 | rs2640779 | C/G |
| | | | 11 | 108509254 | rs3741060 | A/G |
| | | | 11 | 108507480 | rs4643041 | T/G |
| EIF5B-201 | ENST00000289371.11 | NM_015904.4 | 2 | 99361585 | rs12233086 | G/A |
| | | | 2 | 99376359 | rs7558074 | A/C |
| | | | 2 | 99379054 | rs11896520 | C/G |
| | | | 2 | 99379341 | rs1376443 | T/A |
| | | | 2 | 99400250 | rs2290257 | G/C |
| | | | 2 | 99400386 | rs9308819 | G/A |
| | | | 2 | 99400611 | rs2290258 | A/C |
| TRPV2-201 | ENST00000338560.12 | NM_016113.5 | 17 | 16415658 | rs77272400 | C/A |
| | | | 17 | 16422691 | rs1129235 | A/C |
| | | | 17 | 16422654 | rs8121 | A/G |
| | | | 17 | 16421619 | rs14039 | C/G |
| HECA-201 | ENST00000367658.3 | NM_016217.3 | 6 | 139166699 | rs3173082 | T/A |
| | | | 6 | 139178038 | rs3777668 | A/G |
| | | | 6 | 139178865 | rs7745062 | T/C |
| | | | 6 | 139178998 | rs7745259 | T/C |
| | | | 6 | 139179605 | rs3777667 | G/A |
| | | | 6 | 139180204 | rs8476 | G/T |
| ZRANB1-201 | ENST00000359653.4 | NM_017580.3 | 10 | 124981787 | rs17152433 | G/A |
| | | | 10 | 124985429 | rs3906796 | A/C |
| | | | 10 | 124985524 | rs3845 | T/A |
| | | | 10 | 124986120 | rs1046373 | A/C |
| | | | 10 | 124986291 | rs142478896 | G/A |
| | | | 10 | 124986287 | rs143258620 | G/A |
| | | | 10 | 124986289 | rs145108464 | G/A |
| | | | 10 | 124986285 | rs148576335 | G/A |
| | | | 10 | 124987076 | rs17152448 | C/G |
| | | | 10 | 124988117 | rs3740535 | T/A |
| MREG-201 | ENST00000263268.11 | NM_018000.3 | 2 | 216013285 | rs1864253 | C/A |
| | | | 2 | 215943631 | rs150285041 | G/A |
| | | | 2 | 215943429 | rs6435927 | C/A |
| UBC-201 | ENST00000339647.6 | NM_021009.7 | 12 | 124913493 | rs1071862 | G/A |
| RMND5A-201 | ENST00000283632.5 | NM_022780.4 | 2 | 86720660 | rs76239019 | C/T |
| | | | 2 | 86751970 | rs10186663 | G/A |
| | | | 2 | 86775106 | rs1193 | C/T |
| | | | 2 | 86775583 | rs13297 | G/A |
| | | | 2 | 86775905 | rs1049457 | C/A |
| | | | 2 | 86776074 | rs1049458 | C/A |
| | | | 2 | 86776723 | rs11547281 | C/A |
| PLA2G2F-201 | ENST00000375102.4 | NM_022819.4 | 1 | 20148414 | rs3738126 | T/C |
| | | | 1 | 20149898 | rs818571 | C/A |
| MMRN2-201 | ENST00000372027.10 | NM_024756.3 | 10 | 86957397 | rs3750823 | C/A |
| | | | 10 | 86942633 | rs10887673 | G/A |
| CTC1-214 | ENST00000651323.1 | NM_025099.6 | 17 | 8236145 | rs74506939 | G/A |
| | | | 17 | 8229445 | rs3826543 | T/C |
| KRTAP1-1-201 | ENST00000306271.5 | NM_030967.3 | 17 | 41041386 | rs112542604 | A/G |
| | | | 17 | 41041362 | rs141856614 | G/A |
| | | | 17 | 41040924 | rs3815561 | G/A |
| SFXN3-201 | ENST00000393459.6 | NM_030971.6 | 10 | 101032337 | rs3740497 | C/G |
| | | | 10 | 101040603 | rs807048 | A/G |
| KRTAP3-2-201 | ENST00000391587.3 | NM_031959.3 | 17 | 40999775 | rs3829598 | G/A |
| | | | 17 | 40999815 | rs9890989 | A/G |
| | | | 17 | 40999832 | rs9897046 | T/C |
| | | | 17 | 40999629 | rs3813048 | C/T |
| | | | 17 | 40999717 | rs3813050 | A/G |
| | | | 17 | 40999506 | rs3813047 | G/A |
| KRTAP4-8-201 | ENST00000333822.5 | NM_031960.3 | 17 | 41097890 | rs375280428 | A/G |
| | | | 17 | 41097717 | rs147906218 | T/C |
| | | | 17 | 41097704 | rs76327324 | G/A |
| | | | 17 | 41097708 | rs80168553 | C/A |
| | | | 17 | 41097583 | rs72625995 | C/A |
| | | | 17 | 41097563 | rs77163010 | G/A |
| | | | 17 | 41097433 | rs7212517 | A/C |
| | | | 17 | 41097497 | rs72625994 | T/C |
| | | | 17 | 41097269 | rs6503593 | A/C |
| | | | 17 | 41097329 | rs8067655 | G/T |
| CGNL1-201 | ENST00000281282.6 | NM_032866.5 | 15 | 57439375 | rs7182648 | C/A |
| | | | 15 | 57439530 | rs1280396 | A/C |
| | | | 15 | 57543705 | rs1620402 | T/A |
| | | | 15 | 57547389 | rs16977594 | A/C |
| | | | 15 | 57547590 | rs2131730 | A/T |
| | | | 15 | 57547633 | rs2131731 | A/G |
| | | | 15 | 57547929 | rs1666573 | T/C |
| | | | 15 | 57548130 | rs2934418 | A/C |
| | | | 15 | 57548538 | rs3742966 | C/A |
| | | | 15 | 57548571 | rs937248 | G/A |
| | | | 15 | 57549412 | rs3742963 | T/C |
| | | | 15 | 57549393 | rs3742964 | A/T |
| | | | 15 | 57549610 | rs7166305 | C/A |
| | | | 15 | 57550074 | rs17820431 | A/G |
| | | | 15 | 57550116 | rs17820437 | T/C |
| | | | 15 | 57419643 | rs11856 | A/C |
| | | | 15 | 57550264 | rs8784 | T/A |
| | | | 15 | 57550343 | rs12439292 | T/G |
| KRTAP3-3-201 | ENST00000391586.3 | NM_033185.3 | 17 | 40994105 | rs1989365 | A/G |
| | | | 17 | 40994032 | rs2010201 | A/G |
| | | | 17 | 40993915 | rs12452295 | G/A |
| | | | 17 | 40993489 | rs8072775 | T/C |
| RBM33-207 | ENST00000401878.8 | NM_053043.3 | 7 | 155644781 | rs7786826 | T/C |
| | | | 7 | 155711226 | rs7800753 | A/C |
| | | | 7 | 155738260 | rs2178428 | T/C |
| | | | 7 | 155738386 | rs3735576 | A/G |
| | | | 7 | 155775262 | rs2108192 | C/G |
| | | | 7 | 155775856 | rs10269841 | A/T |
| | | | 7 | 155776473 | rs3801904 | C/T |
| | | | 7 | 155777289 | rs2007922 | G/A |
| | | | 7 | 155778909 | rs3807890 | A/G |
| | | | 7 | 155779115 | rs11520636 | C/T |
| LRRC15-201 | ENST00000347624.4 | NM_130830.5 | 3 | 194360906 | rs923935 | T/C |
| | | | 3 | 194360663 | rs923936 | G/A |
| CAPN12-201 | ENST00000328867.9 | NM_144691.4 | 19 | 38744239 | rs76443569 | T/C |
| | | | 19 | 38742506 | rs936524 | A/G |
| | | | 19 | 38738449 | rs12983010 | A/G |
| | | | 19 | 38737604 | rs73038948 | T/C |
| | | | 19 | 38733773 | rs4801861 | A/G |
| | | | 19 | 38730655 | rs1060186 | A/C |
| KRT6C-201 | ENST00000252250.7 | NM_173086.5 | 12 | 52473557 | rs201833096 | G/A |
| | | | 12 | 52473555 | rs2885799 | C/A |
| | | | 12 | 52473476 | rs411107 | C/T |
| | | | 12 | 52473540 | rs419374 | G/T |
| | | | 12 | 52473558 | rs556681459 | A/G |
| | | | 12 | 52472276 | rs11608915 | C/G |
| | | | 12 | 52469837 | rs382367 | T/C |
| | | | 12 | 52469780 | rs3894847 | C/T |
| | | | 12 | 52469429 | rs412533 | C/T |
| | | | 12 | 52469254 | rs410562 | G/A |
| | | | 12 | 52469260 | rs570463693 | A/C |
| | | | 12 | 52468986 | rs1053796 | T/C |
| | | | 12 | 52468983 | rs2567 | G/C |
| | | | 12 | 52468999 | rs2568 | G/A |
| | | | 12 | 52468830 | rs583678 | T/A |
| | | | 12 | 52468741 | rs372790 | A/G |
| | | | 12 | 52468719 | rs432498 | C/A |
| RASSF3-203 | ENST00000542104.6 | NM_178169.4 | 12 | 64610607 | rs3741748 | C/G |
| | | | 12 | 64610740 | rs77813527 | A/C |
| | | | 12 | 64695505 | rs1797683 | T/A |
| | | | 12 | 64697251 | rs8648 | G/A |
| LEMD2-201 | ENST00000293760.10 | NM_181336.4 | 6 | 33772548 | rs11755593 | G/A |
| | | | 6 | 33772605 | rs2296744 | G/A |
| | | | 6 | 33772183 | rs10947436 | A/G |
| | | | 6 | 33772248 | rs2296743 | C/A |
| | | | 6 | 33771954 | rs367875497 | T/C |
| KRTAP7-1-201 | ENST00000621162.1 | NM_181606.3 | 21 | 30829633 | rs9982775 | A/G |
| | | | 21 | 30829517 | rs9982675 | A/C |
| | | | 21 | 30829548 | rs9982755 | A/G |
| | | | 21 | 30829504 | rs9982910 | G/A |
| KRTAP12-2-201 | ENST00000360770.3 | NM_181684.3 | 21 | 44666841 | rs13046903 | C/G |
| | | | 21 | 44666801 | rs7275281 | G/A |
| | | | 21 | 44666840 | , rs7275298 | G/A |
| | | | 21 | 44666802 | rs7276859 | A/G |
| | | | 21 | 44666490 | rs12481809 | C/T |
| | | | 21 | 44666540 | rs12483730 | G/A |
| | | | 21 | 44666460 | rs2838622 | A/C |
| KRTAP12-1-201 | ENST00000391617.1 | NM_181686.2 | 21 | 44682100 | rs7283084 | C/T |
| | | | 21 | 44682024 | rs9984476 | C/A |
| | | | 21 | 44681773 | rs2838621 | C/A |
| SCRIB-202 | ENST00000356994.7 | NM_182706.5 | 8 | 143810914 | rs6558394 | G/A |
| | | | 8 | 143807002 | rs10866915 | A/C |
| ARRDC4-201 | ENST00000268042.7 | NM_183376.3 | 15 | 97960870 | rs4965046 | G/A |
| | | | 15 | 97961092 | rs112100339 | C/G |
| | | | 15 | 97961096 | rs12101554 | A/C |
| | | | 15 | 97970615 | rs2130882 | T/C |
| | | | 15 | 97971259 | rs3803385 | A/G |
| | | | 15 | 97971819 | rs3169159 | G/A |
| | | | 15 | 97971810 | rs3169160 | C/T |
| | | | 15 | 97971991 | rs9652498 | A/C |
| | | | 15 | 97972903 | rs1043372 | C/A |
| | | | 15 | 97973030 | rs1062702 | C/G |
| | | | 15 | 97973392 | rs1043374 | A/C |
| | | | 15 | 97973522 | rs1043382 | T/A |
| KRTAP10-6-201 | ENST00000400368.1 | NM_198688.3 | 21 | 44592300 | rs201732843 | C/A |
| | | | 21 | 44592301 | rs233309 | G/A |
| | | | 21 | 44592106 | rs13051603 | A/G |
| | | | 21 | 44591806 | rs58757665 | C/G |
| | | | 21 | 44591836 | rs9980630 | T/A |
| | | | 21 | 44591702 | rs233303 | G/A |
| KRTAP26-1-201 | ENST00000360542.5 | NM_203405.2 | 21 | 30320223 | rs78731676 | C/T |
| | | | 21 | 30320059 | rs3804008 | A/C |
| | | | 21 | 30319959 | rs3804007 | G/T |
| | | | 21 | 30319253 | rs2236427 | C/A |

TABLE 2
Primer sequences for multiplex amplification system

| | | | Upstream | Downstream | Amplicon |
|---|---|---|---|---|---|
| | Chromo- | Target | primer | primer | size |
| NM number | some | site | (5'-3') | (5'-3') | (bp) |
| NM_000081.4 | 1 | rs11464 | ATGGCAACCCTTGGA | TCCATACCAGTGGAA | 139 |
| | | | CAGTA | GGAGAGA | |
| | | | (SEQ ID NO: 1) | (SEQ ID NO: 2) | |
| NM_000081.4 | 1 | rs1063128 | CGTCATAGAGCCCTT | TGATGCTGAATGAAT | 137 |
| | | | CTTTTACCT | TCTTGAACC | |
| | | | (SEQ ID NO: 3) | (SEQ ID NO: 4) | |
| NM_000081.4 | 1 | rs3820553 | TTATCCTGAGCGGTG | GGATCCATACAACAG | 138 |
| | | | CTGTT | CATATTCCAA | |
| | | | (SEQ ID NO: 5) | (SEQ ID NO: 6) | |
| NM_000081.4 | 1 | rs6696123 | GCTTCCAAGAAAAAC | AGCTCCGTTGAAGAG | 139 |
| | | | AAGTTTGTCA | AAGCAA | |
| | | | (SEQ ID NO: 7) | (SEQ ID NO: 8) | |
| NM_000081.4 | 1 | rs2273584 | ACTCTCTCAAAGACC | ACAAATATGCCAGTT | 138 |
| | | | CTTCAGC | CCACCAATTT | |
| | | | (SEQ ID NO: 9) | (SEQ ID NO: 10) | |
| NM_001014342.3 | 1 | rs3818831 | ACAAATACACCAAGC | CCAGCATATGCATGA | 138 |
| | | | AAGATGGG | TGACATCC | |
| | | | (SEQ ID NO: 11) | (SEQ ID NO: 12) | |
| NM_001101662.2 | 1 | rs1770791, | ATGATAACTGAGCAG | CGTCCATCAAAGCCT | 136 |
| | | rs8375 | TTGAAGAAGA | GGTAC | |
| | | | (SEQ ID NO: 13) | (SEQ ID NO: 14) | |
| NM_001101662.2 | 1 | rs11205896 | TGAAGTTGAAGCTGT | CTTGAGCGTCTCAGC | 139 |
| | | | TGATAGTGAAT | ATTTCC | |
| | | | (SEQ ID NO: 15) | (SEQ ID NO: 16) | |
| NM_001195571.2 | 1 | rs10788862 | CGGTGTTTCAAAAAT | TGGTTGTTTGGATTC | 138 |
| | | | CAAAAATTTGGT | AATCGTTTACA | |
| | | | (SEQ ID NO: 17) | (SEQ ID NO: 18) | |
| NM_022819.4 | 1 | rs818571 | ACCTGCCCGAAAACC | ATGTCCCTTCTGCCT | 139 |
| | | | ACTG | CCTTC | |
| | | | (SEQ ID NO: 19) | (SEQ ID NO: 20) | |
| NM_022819.4 | 1 | rs3738126 | CCAACTGCAGCATCT | ACCTCGTCTGGTCCC | 140 |
| | | | ATGAACC | CAA | |
| | | | (SEQ ID NO: 21) | (SEQ ID NO: 22) | |
| NM_001395404.1 | 2 | rs6436712 | GTCTGTGGCAGCTGC | CCACATGAGCGGCAG | 140 |
| | | | ACC | GTG | |
| | | | (SEQ ID NO: 23) | (SEQ ID NO: 24) | |
| NM_001395405.1 | 2 | rs112558913, | GTGGAAGTTGTGGTG | GTAGCACCTGCAGGT | 131 |
| | | rs113140754 | GCTGT | GGT | |
| | | | (SEQ ID NO: 25) | (SEQ ID NO: 26) | |
| NM_001395407.1 | 2 | rs113823535 | AGCACGCCCGTGATC | CTAGCAGCAGCATTG | 135 |
| | | | TGT | CTTCTT | |
| | | | (SEQ ID NO: 27) | (SEQ ID NO: 28) | |
| NM_002539.3 | 2 | rs2302613, | CACGCAGAGGGATTT | TGGCAGTCAAACTCT | 138 |
| | | rs2302614 | GGAATT | TCATTACC | |
| | | | (SEQ ID NO: 29) | (SEQ ID NO: 30) | |
| NM_002539.3 | 2 | rs1049500 | GCTGCTGCCTCTACG | GCTGGCATCCTGTTC | 132 |
| | | | TTCAAT | CTCTA | |
| | | | (SEQ ID NO: 31) | (SEQ ID NO: 32) | |
| NM_002709.3 | 2 | rs7475 | GCTACCATAGATGTG | TGCACTGTGCTGAAA | 137 |
| | | | ACATTTTTGACC | ATGAAATATCT | |
| | | | (SEQ ID NO: 33) | (SEQ ID NO: 34) | |
| NM_002709.3 | 2 | rs11687800 | TGATGCTCTATTGTG | AGCATGCCAAAAGGT | 131 |
| | | | CAGTAGC | GTTCC | |
| | | | (SEQ ID NO: 35) | (SEQ ID NO: 36) | |
| NM_002709.3 | 2 | rs1128416 | CTCAGCCAGCCTATT | TCCTAAGAAAAGATA | 135 |
| | | | CTTTTGG | GTTGGCTTCTG | |
| | | | (SEQ ID NO: 37) | (SEQ ID NO: 38) | |
| NM_002709.3 | 2 | rs3190 | AAATTGGGACAACAT | CCAATCACAATTGTA | 137 |
| | | | ACTTTACGC | TCTAAGTTTCACTTT | |
| | | | (SEQ ID NO: 39) | T | |
| | | | | (SEQ ID NO: 40) | |
| NM_014585.6 | 2 | rs2304704 | ACATTTGGCTCCCCA | TCCTCTTCTTTAAGA | 140 |
| | | | GTCATC | CCAGCTTTCA | |
| | | | (SEQ ID NO: 41) | (SEQ ID NO: 42) | |
| NM_015904.4 | 2 | rs11896520 | TCTGATGATGATCGG | ACATGCCCAAGTACG | 137 |
| | | | ACTAAAGAAGA | CAGAT | |
| | | | (SEQ ID NO: 43) | (SEQ ID NO: 44) | |
| NM_015904.4 | 2 | rs2290257 | CTACCTTGTATGCCA | AGGACAAAACAGATC | 136 |
| | | | GCACCT | AAGATTAAAACAG | |
| | | | (SEQ ID NO: 45) | (SEQ ID NO: 46) | |
| NM_015904.4 | 2 | rs1376443 | GGCATGTGGACACAG | GTCTGTTCATTAATA | 133 |
| | | | GGAA | GCTTCAAGAGGAA | |
| | | | (SEQ ID NO: 47) | (SEQ ID NO: 48) | |
| NM_015904.4 | 2 | rs12233086 | ACAAGCCAGGTCCTA | CGCAGTTTCGCTTTT | 136 |
| | | | ACATAGA | TCTTCATC | |
| | | | (SEQ ID NO: 49) | (SEQ ID NO: 50) | |
| NM_015904.4 | 2 | rs9308819 | TCCTGATATTTGGGC | TGGAACTTTTTCCTC | 136 |
| | | | TTAGTGCTT | AAACAAACAC | |
| | | | (SEQ ID NO: 51) | (SEQ ID NO: 52) | |
| NM_015904.4 | 2 | rs7558074 | AGGATGCTGGATTGG | TTCTTCTTCCTCTTC | 134 |
| | | | ATGATTG | TTCCTCCT | |
| | | | (SEQ ID NO: 53) | (SEQ ID NO: 54) | |
| NM_015904.4 | 2 | rs2290258 | TCAAAGTTTCTCAAT | CATGCGGAGAAATCT | 137 |
| | | | GGCCAGT | ACATATTTTGAA | |
| | | | (SEQ ID NO: 55) | (SEQ ID NO: 56) | |
| NM_018000.3 | 2 | rs1864253 | CTGAGGGACTGGCTG | TCATCATCCCTCACC | 140 |
| | | | AGAAC | AGAGTTG | |
| | | | (SEQ ID NO: 57) | (SEQ ID NO: 58) | |
| NM_018000.3 | 2 | rs6435927 | GCGAGATCCGTGTTC | ACATGAGGCAAAGAT | 136 |
| | | | TCCATT | TAAGCACT | |
| | | | (SEQ ID NO: 59) | (SEQ ID NO: 60) | |
| NM_018000.3 | 2 | rs150285041 | TGGCATTTTATCTGG | TGCATATGTGCAAGT | 139 |
| | | | AATAGCATCTG | CTGCATG | |
| | | | (SEQ ID NO: 61) | (SEQ ID NO: 62) | |
| NM_022780.4 | 2 | rs13297 | CAAAAGCCAGAGCTC | CACAGAAAGCTGCAG | 135 |
| | | | AGCTTTA | AGTCC | |
| | | | (SEQ ID NO: 63) | (SEQ ID NO: 64) | |
| NM_022780.4 | 2 | rs10186663 | TGATTCTGACATTAG | GACCAGATTCCTGAC | 140 |
| | | | CAGTGTGG | AGAGCT | |
| | | | (SEQ ID NO: 65) | (SEQ ID NO: 66) | |
| NM_022780.4 | 2 | rs76239019 | GAACGGCTGCGGACA | GCTCGCACAGCTGCC | 131 |
| | | | CCTG | CGT | |
| | | | (SEQ ID NO: 67) | (SEQ ID NO: 68) | |
| NM_022780.4 | 2 | rs1049457 | CCAGACCTTAACCAA | AGAGAGACAAAAAGC | 137 |
| | | | AGATGCT | ATCCTCCT | |
| | | | (SEQ ID NO: 69) | (SEQ ID NO: 70) | |
| NM_022780.4 | 2 | rs1049458 | AGGCAACGTTTATAA | GGTTTAGCATAGTCC | 134 |
| | | | CAGTCACAT | CATGGC | |
| | | | (SEQ ID NO: 71) | (SEQ ID NO: 72) | |
| NM_022780.4 | 2 | rs1193 | CTATGTGGATGGCTA | GGATTCCAACAGTGA | 138 |
| | | | CTCCGT | AGCAGAA | |
| | | | (SEQ ID NO: 73) | (SEQ ID NO: 74) | |
| NM_022780.4 | 2 | rs11547281 | ACATTAGCCCTCTGG | CTAGTGATGAAACTA | 137 |
| | | | TTTTCCA | ACCAAACTATGC | |
| | | | (SEQ ID NO: 75) | (SEQ ID NO: 76) | |
| NM_001354604.2 | 3 | rs576 | CCCATTCTTTCAACC | CCCCCATATAAATAT | 140 |
| | | | CCCTTAAC | GAAGAGAAAAATAGA | |
| | | | (SEQ ID NO: 77) | CA | |
| | | | | (SEQ ID NO: 78) | |
| NM_001354604.2 | 3 | rs9863910 | AATCCTCCCTGCACT | CCTGGGCTATTGATA | 134 |
| | | | GCATT | AAGCATGAAA | |
| | | | (SEQ ID NO: 79) | (SEQ ID NO: 80) | |
| NM_001354604.2 | 3 | rs704246 | GGAGAATGCTGCAAT | AGTGGTGTGGATCAT | 140 |
| | | | CTTGGG | TTTCTTGTTAC | |
| | | | (SEQ ID NO: 81) | (SEQ ID NO: 82) | |
| NM_130830.5 | 3 | rs923936 | TATCTCAGCCTCGCC | GGTTGCTGCACTGGG | 139 |
| | | | AACAA | AGAA | |
| | | | (SEQ ID NO: 83) | (SEQ ID NO: 84) | |
| NM_130830.5 | 3 | rs923935 | GCTGCCCTAGCGAGT | CATTGAGTTCAGTGA | 138 |
| | | | GTAC | TGTGCGT | |
| | | | (SEQ ID NO: 85) | (SEQ ID NO: 86) | |
| NM_001371242.2 | 6 | rs3747790 | GCCTTTCATTTTTGG | GTTCCAGTTCTCTCC | 140 |
| | | | TAGCTGCTT | TGTCACT | |
| | | | (SEQ ID NO: 87) | (SEQ ID NO: 88) | |
| NM_001371242.2 | 6 | rs1135384, | TGGGACTATAGGCAC | CGAGGCGAGTGAATT | 123 |
| | | rs6922741, | ACATCAC | GCTTG | |
| | | rs9486407 | (SEQ ID NO: 89) | (SEQ ID NO: 90) | |
| NM_001371242.2 | 6 | rs9320182 | CCAGCTTTAAATTAT | TGATCCACCATGTCA | 138 |
| | | | CTCTTCATGCC | GTCTCC | |
| | | | (SEQ ID NO: 91) | (SEQ ID NO: 92) | |
| NM_001371242.2 | 6 | rs1057429 | GGAAAGCCTTAGGTA | ACTCAAATCCTACTA | 133 |
| | | | TTCACACATTTAA | ACAAATGTTACCA | |
| | | | (SEQ ID NO: 93) | (SEQ ID NO: 94) | |
| NM_001371242.2 | 6 | rs13319 | TCTTGTCCTCCTATC | AGTGGCTATAGTGCC | 131 |
| | | | CACCTG | CCCAA | |
| | | | (SEQ ID NO: 95) | (SEQ ID NO: 96) | |
| NM_001371242.2 | 6 | rs11152999 | CCTCGGCGGAAAAGA | CTGTTTGCTCCGGTC | 134 |
| | | | AAGTG | CGAC | |
| | | | (SEQ ID NO: 97) | (SEQ ID NO: 98) | |
| NM_001371242.2 | 6 | rs11758586 | AGAGTCAGCTAGCAG | ACTCATCCTCGTTTC | 138 |
| | | | ATTAGAAAC | CACTCAG | |
| | | | (SEQ ID NO: 99) | (SEQ ID NO: 100) | |
| NM_001371242.2 | 6 | rs1340623, | CCCTAACGGAGCCCC | CGCGGAGCTGGCCGT | 139 |
| | | rs4945755, | CAGTG | GTG | |
| | | rs4946759 | (SEQ ID NO: 101) | (SEQ ID NO: 102) | |
| NM_001371242.2 | 6 | rs3747787 | CCTGGGCCTTCTCTT | CACAGGCAAAACACA | 138 |
| | | | TCACT | TCCTTTATG | |
| | | | (SEQ ID NO: 103) | (SEQ ID NO: 104) | |
| NM_001371242.2 | 6 | rs8013 | TCTCTGGAGTCTATG | CACAAAAGGCCTAAA | 138 |
| | | | GTAGGCA | ATCAGAGC | |
| | | | (SEQ ID NO: 105) | (SEQ ID NO: 106) | |
| NM_001371242.2 | 6 | rs4946758 | GGCACTCTATTCACT | TGCTCCTCTCACTCT | 139 |
| | | | GCAGGA | CTCTCT | |
| | | | (SEQ ID NO: 107) | (SEQ ID NO: 108) | |
| NM_001371242.2 | 6 | rs1057433 | CAAGCAATCTTGGAA | AAATGTTTAGCACTT | 136 |
| | | | TCCTCAACT | TTGTTCAAAGC | |
| | | | (SEQ ID NO: 109) | (SEQ ID NO: 110) | |
| NM_001371242.2 | 6 | rs6933677 | TGGTATTTACACCTG | GCTTGGTTTATTCCT | 138 |
| | | | GAAAATCAGA | GTTAGACACTC | |
| | | | (SEQ ID NO: 111) | (SEQ ID NO: 112) | |
| NM_001371242.2 | 6 | rs11537704 | AGACAAAGGCTTAAG | GGCTTTCCCATTGTG | 139 |
| | | | TTTGAAAGGTAG | CTATGC | |
| | | | (SEQ ID NO: 113) | (SEQ ID NO: 114) | |
| NM_001371242.2 | 6 | rs2297970 | AAATCCTGACTTCAC | TCTCCTTGGGCCAGT | 136 |
| | | | AGGAGAAC | GAAAG | |
| | | | (SEQ ID NO: 115) | (SEQ ID NO: 116) | |
| NM_001371242.2 | 6 | rs9320183, | AGCTAGTAAAACAGA | CACACCATTTTGGCC | 139 |
| | | rs9486408, | AGGTGCCA | AGTAATTG | |
| | | rs9486409 | (SEQ ID NO: 117) | (SEQ ID NO: 118) | |
| NM_001371242.2 | 6 | rs1159148 | GCAACACTGCCACCA | AATTCAGCTTTGCCC | 132 |
| | | | AAATC | TCCCA | |
| | | | (SEQ ID NO: 119) | (SEQ ID NO: 120) | |
| NM_003980.6 | 6 | rs1681 | GAGTCGCGCAGGCTG | CTTCACGAACGCGAG | 142 |
| | | | GAAG | CTTCT | |
| | | | (SEQ ID NO: 121) | (SEQ ID NO: 122) | |
| NM_003980.6 | 6 | rs2076190 | GGGTTTTCCCTGTTT | GTCAAACATTTTATT | 125 |
| | | | TCAGTTGT | TGGTGTTTCATGC | |
| | | | (SEQ ID NO: 123) | (SEQ ID NO: 124) | |
| NM_004415.4 | 6 | rs2076300 | GCCATGAATAAGGAA | CATTGAAATAGCCCC | 139 |
| | | | CTCATCGAAAA | TCTTATATGCT | |
| | | | (SEQ ID NO: 125) | (SEQ ID NO: 126) | |
| NM_004415.4 | 6 | rs2076304 | TTCGCAGGCAGATAG | TCAGCAATTGCTTGT | 139 |
| | | | AGCAC | GAATCATTCT | |
| | | | (SEQ ID NO: 127) | (SEQ ID NO: 128) | |
| NM_004415.4 | 6 | rs6929069 | TCACAGAGAACCTGA | TGGCTCCTTAGTTCC | 139 |
| | | | CCAAGG | AAGATGG | |
| | | | (SEQ ID NO: 129) | (SEQ ID NO: 130) | |
| NM_004415.4 | 6 | rs2076299 | TGATCGACAAAGAAA | TCTTGCTCCTGAACC | 136 |
| | | | CAAATGACC | TTCAGT | |
| | | | (SEQ ID NO: 131) | (SEQ ID NO: 132) | |
| NM_004415.4 | 6 | rs12250 | AGTAAAGGCTGTTCT | AGAAGAGAAGAAACA | 134 |
| | | | GGCTTTT | AATCCTAAGTTCG | |
| | | | (SEQ ID NO: 133) | (SEQ ID NO: 134) | |
| NM_004415.4 | 6 | rs2064217 | AAGAACTTGCACAGT | AGCAGAGTTTCCAGT | 137 |
| | | | GAAATATCTG | CCTGAG | |
| | | | (SEQ ID NO: 135) | (SEQ ID NO: 136) | |
| NM_004415.4 | 6 | rs2744380 | GCTTACCCAGCCCTT | AAGAATTCCCTGTGG | 135 |
| | | | ACAAC | CGTCA | |
| | | | (SEQ ID NO: 137) | (SEQ ID NO: 138) | |
| NM_014069.3 | 6 | rs2074478 | TTCTGTTCCCAGGCA | GAGAACACCACCTTA | 139 |
| | | | TCTCC | CGGGTT | |
| | | | (SEQ ID NO: 139) | (SEQ ID NO: 140) | |
| NM_014069.3 | 6 | rs1265099, | TACTCTCCTACCTCG | TGTCTACTGATAGTT | 141 |
| | | rs3132558 | CCTCC | CCTGCCG | |
| | | | (SEQ ID NO: 141) | (SEQ ID NO: 142) | |
| NM_016217.3 | 6 | rs3777668 | AGCCACAGAATCTAA | TCCCTTCCCTCTTAA | 135 |
| | | | CTGGTGAG | GGTCAAA | |
| | | | (SEQ ID NO: 143) | (SEQ ID NO: 144) | |
| NM_016217.3 | 6 | rs3777667 | ATGCTAAAGTAAGGA | TCCCAAATCAGCCAG | 130 |
| | | | TTCTGTGGAT | ATTTTCC | |
| | | | (SEQ ID NO: 145) | (SEQ ID NO: 146) | |
| NM_016217.3 | 6 | rs7745062 | CAGCGTTTCACCATG | AATACTTCATATAGA | 135 |
| | | | TTAGCTAG | ACTGAAACTTGGC | |
| | | | (SEQ ID NO: 147) | (SEQ ID NO: 148) | |
| NM_016217.3 | 6 | rs3173082 | TCTGGCTCCGAGAAG | GGGAGTTCTGCCGGT | 139 |
| | | | AACAC | CCAT | |
| | | | (SEQ ID NO: 149) | (SEQ ID NO: 150) | |
| NM_016217.3 | 6 | rs7745259 | TCTTTCAGTTTGGAT | GTTGCATCAAACACT | 136 |
| | | | GATTTGGTTTG | GTCAGTTC | |
| | | | (SEQ ID NO: 151) | (SEQ ID NO: 152) | |
| NM_016217.3 | 6 | rs8476 | GGGTGAATTATCCCA | TTTTAGATGAGTTAA | 135 |
| | | | TTCTAGTTACAG | TTTTCCCCCAAC | |
| | | | (SEQ ID NO: 153) | (SEQ ID NO: 154) | |
| NM_181336.4 | 6 | rs10947436, | CCAGGGAGTCTGTCA | AGCACAGGCTTTCTC | 132 |
| | | rs2296743 | CCTG | CCTTC | |
| | | | (SEQ ID NO: 155) | (SEQ ID NO: 156) | |
| NM_181336.4 | 6 | rs367875497 | CTCAGGTCAGGAGGA | CTGTGAGAAGCAACA | 133 |
| | | | GTGAG | CTGAGG | |
| | | | (SEQ ID NO: 157) | (SEQ ID NO: 158) | |
| NM_181336.4 | 6 | rs11755593, | TCTGACTCAGAGCGA | AGGACGAGACTGAAA | 139 |
| | | rs2296744 | TAAGCC | GTCA | |
| | | | | AGG | |
| | | | (SEQ ID NO: 159) | (SEQ ID NO: 160) | |
| NM_053043.3 | 7 | rs2178428 | CAGAGTCCTCTACCA | AGGCTGGCTAAAGTG | 138 |
| | | | TTCACTCAG | CAGAA | |
| | | | (SEQ ID NO: 161) | (SEQ ID NO: 162) | |
| NM_053043.3 | 7 | rs11520636 | CCTTTGTGGGTGACT | GGAAGCCAACCTGGG | 134 |
| | | | TCAGG | TCTAAA | |
| | | | (SEQ ID NO: 163) | (SEQ ID NO: 164) | |
| NM_053043.3 | 7 | rs2108192 | CAGGCTGGAGTTGGT | TCGATCATAGAGTCA | 136 |
| | | | GTTAGA | GACCACA | |
| | | | (SEQ ID NO: 165) | (SEQ ID NO: 166) | |
| NM_053043.3 | 7 | rs10269841 | CCTAGTTCCCACCAC | TGGCCACCAAATTCC | 139 |
| | | | ACATGA | CAAAG | |
| | | | (SEQ ID NO: 167) | (SEQ ID NO: 168) | |
| NM_053043.3 | 7 | rs2007922 | CTGGAAGGTCTGTTT | GCCTGCATGACTTTG | 138 |
| | | | TGGTGTTA | CCAATC | |
| | | | (SEQ ID NO: 169) | (SEQ ID NO: 170) | |
| NM_053043.3 | 7 | rs7786826 | GCCATGTTGCGGTAG | CCGGGTACGGGTGAC | 130 |
| | | | TTTGTTG | GGA | |
| | | | (SEQ ID NO: 171) | (SEQ ID NO: 172) | |
| NM_053043.3 | 7 | rs3801904 | GGGCTCTCAGCCTCC | AGACCTGCTTCAGCT | 135 |
| | | | TAGA | CTTCA | |
| | | | (SEQ ID NO: 173) | (SEQ ID NO: 174) | |
| NM_053043.3 | 7 | rs7800753 | TGCTTCCTACACAGC | GGTGCTGCACCGGAA | 126 |
| | | | CTCCT | GCA | |
| | | | (SEQ ID NO: 175) | (SEQ ID NO: 176) | |
| NM_053043.3 | 7 | rs3735576 | GGCCCTTCATTCCTC | CTGAGGTTGCTGAGG | 140 |
| | | | CTAGA | CTGAG | |
| | | | (SEQ ID NO: 177) | (SEQ ID NO: 178) | |
| NM_053043.3 | 7 | rs3807890 | TGTGCTCATAGACCA | CCGCAGGGACTAGAG | 139 |
| | | | TCAGACG | AGTTTC | |
| | | | (SEQ ID NO: 179) | (SEQ ID NO: 180) | |
| NM_002568.4 | 8 | rs201017624 | AAGTGTACGAGCTGT | GGTCTTAGTTGAGCA | 138 |
| | | | TCCCA | ATTTGGCTA | |
| | | | (SEQ ID NO: 181) | (SEQ ID NO: 182) | |
| NM_014781.5 | 8 | rs17337252 | CAGAGAATGTTTGGG | GGGAAATGAGGTTAA | 139 |
| | | | AAGACTGG | CAAAGATTCGT | |
| | | | (SEQ ID NO: 183) | (SEQ ID NO: 184) | |
| NM_014781.5 | 8 | rs2305427 | ACTTTCTGAAGATCG | TTCAGGTGCACAAGC | 139 |
| | | | AGCTCGT | TCCAT | |
| | | | (SEQ ID NO: 185) | (SEQ ID NO: 186) | |
| NM_014781.5 | 8 | rs1129660 | TCTACCTGCCCTGGA | GTCCCCAAAGGAACT | 138 |
| | | | TCTCA | TTAAATCTGT | |
| | | | (SEQ ID NO: 187) | (SEQ ID NO: 188) | |
| NM_182706.5 | 8 | rs10866915 | CGTTTGACCAGGCCA | TCCCCCTTATAGGGT | 139 |
| | | | ATAACC | GTGGA | |
| | | | (SEQ ID NO: 189) | (SEQ ID NO: 190) | |
| NM_182706.5 | 8 | rs6558394 | GAAGGTGCTCACCTG | AGGAACTGGATGACG | 129 |
| | | | CTACT | CTGAC | |
| | | | (SEQ ID NO: 191) | (SEQ ID NO: 192) | |
| NM_003750.4 | 10 | rs7908387 | TTCATGCATCTACAC | CGTACGCTCAGGAGT | 137 |
| | | | TCCATCG | AATAGGG | |
| | | | (SEQ ID NO: 193) | (SEQ ID NO: 194) | |
| NM_005185.4 | 10 | rs4242797 | GCGTCTCTTCTCTGC | GTGCTCGGTCCTAAG | 130 |
| | | | ACTCC | GTCAG | |
| | | | (SEQ ID NO: 195) | (SEQ ID NO: 196) | |
| NM_005185.4 | 10 | rs4589188, | CTGACTCTGCGGCCC | CTTCCAGTCCAGGAG | 144 |
| | | rs4589189 | TCCA | CCTGC | |
| | | | (SEQ ID NO: 197) | (SEQ ID NO: 198) | |
| NM_005185.4 | 10 | rs1142825 | GAGATCCGCGAGGCC | GATCATCTCGTCCAC | 126 |
| | | | TTC | CTCCT | |
| | | | (SEQ ID NO: 199) | (SEQ ID NO: 200) | |
| NM_005185.4 | 10 | rs1131482 | TGGACTGGAAGCTCC | GGAAGGCAGTGCTGT | 124 |
| | | | CTCC | TTTCA | |
| | | | (SEQ ID NO: 201) | (SEQ ID NO: 202) | |
| NM_014974.3 | 10 | rs10904083 | GCATTAGTAGCACAC | TGTCGAAGGCCTTTA | 140 |
| | | | AGAGATCAGA | CTTTGGA | |
| | | | (SEQ ID NO: 203) | (SEQ ID NO: 204) | |
| NM_014974.3 | 10 | rs3740304 | GCGTCATCCCCATCA | TCTCTAGAAAAGTCC | 139 |
| | | | ACTCC | ATGGAAGCC | |
| | | | (SEQ ID NO: 205) | (SEQ ID NO: 206) | |
| NM_014974.3 | 10 | rs2288681 | CGCAAACAGAGTCCC | GTGAAGGCCCAGGTC | 134 |
| | | | TCAAG | CTTAAA | |
| | | | (SEQ ID NO: 207) | (SEQ ID NO: 208) | |
| NM_014974.3 | 10 | rs4881274 | GCTGGGCGTGGTCAC | ACAGCTTGCCGTAAG | 140 |
| | | | GAA | TGAGG | |
| | | | (SEQ ID NO: 209) | (SEQ ID NO: 210) | |
| NM_017580.3 | 10 | rs17152433 | GGAAAGATTGGGAAT | AAATGTGCGTCTGCT | 138 |
| | | | CATGGTATTCT | CCAAG | |
| | | | (SEQ ID NO: 211) | (SEQ ID NO: 212) | |
| NM_017580.3 | 10 | rs3906796 | TCCAGCATGGGCACT | CGTTTCCAGATCTGA | 127 |
| | | | TAGAA | GACTTAGATG | |
| | | | (SEQ ID NO: 213) | (SEQ ID NO: 214) | |
| NM_017580.3 | 10 | rs142478896, | TGTCCTGATAAGCAC | ACAGCCTTTTTCATC | 139 |
| | | rs143258620, | TTTCTAGACTA | ACAGGGA | |
| | | rs145108464, | (SEQ ID NO: 215) | (SEQ ID NO: 216) | |
| | | rs148576335 | | | |
| NM_017580.3 | 10 | rs3845 | AAACGTTTTGTACCG | CATTCTTAGCTTTTC | 121 |
| | | | ATTATCCAC | TTGCAGTTTGT | |
| | | | (SEQ ID NO: 217) | (SEQ ID NO: 218) | |
| NM_017580.3 | 10 | rs1046373 | GCATGTTGGTTAATT | AGATAGGTCTGAAAT | 137 |
| | | | GTGGCCAT | TTTTACCCAGT | |
| | | | (SEQ ID NO: 219) | (SEQ ID NO: 220) | |
| NM_017580.3 | 10 | rs17152448 | ACCCTGACCACATAG | TTTTGGCCTTTAACA | 133 |
| | | | TGTGATAG | GCCAGT | |
| | | | (SEQ ID NO: 221) | (SEQ ID NO: 222) | |
| NM_017580.3 | 10 | rs3740535 | TCCAGGTCATTTTGC | ACACAAGTCCATCAT | 138 |
| | | | TTTGGG | TATCACGTC | |
| | | | (SEQ ID NO: 223) | (SEQ ID NO: 224) | |
| NM_024756.3 | 10 | rs10887673 | CTGAGCAACGACGTC | CTCCAAGCTGCGCTG | 126 |
| | | | AAGAA | AGT | |
| | | | (SEQ ID NO: 225) | (SEQ ID NO: 226) | |
| NM_024756.3 | 10 | rs3750823 | GCTTCCAGTACTAGC | AGTAAGGTGACCAGC | 137 |
| | | | CTCTCT | TTGGAC | |
| | | | (SEQ ID NO: 227) | (SEQ ID NO: 228) | |
| NM_030971.6 | 10 | rs807048 | ACATCCTCAAGGGCA | GTGAGCCAAGATCGC | 139 |
| | | | GCTAG | ACCAT | |
| | | | (SEQ ID NO: 229) | (SEQ ID NO: 230) | |
| NM_030971.6 | 10 | rs3740497 | GGACGTGAGCCTGAG | CATCACGCGGGACGT | 141 |
| | | | TTTGC | CAC | |
| | | | (SEQ ID NO: 231) | (SEQ ID NO: 232) | |
| NM_001098522.2 | 11 | rs10437608, | CCTTCCGATGGGTCT | CGAGGAACAAGGGCT | 140 |
| | | rs1554998431 | GCTG | GTTTATCT | |
| | | | (SEQ ID NO: 233) | (SEQ ID NO: 234) | |
| NM_001098522.2 | 11 | rs3824886 | AAGAATCTCGCCCAG | TGCTTGTCTCTTGGT | 139 |
| | | | GTGAA | CTCACC | |
| | | | (SEQ ID NO: 235) | (SEQ ID NO: 236) | |
| NM_001098522.2 | 11 | rs3781679 | TGGAACTGCTGGAGA | AGACCCTGAAATTAC | 133 |
| | | | ACAAGG | CGATTTGG | |
| | | | (SEQ ID NO: 237) | (SEQ ID NO: 238) | |
| NM_001098522.2 | 11 | rs16906210 | TACTTCCCCTGATGC | ATGTCAACAATTTTA | 133 |
| | | | CACTG | GAGCAGAGC | |
| | | | (SEQ ID NO: 239) | (SEQ ID NO: 240) | |
| NM_001620.3 | 11 | rs486903 | GGCCCAAAGTGGATA | TTGGGCAGCTTCACA | 142 |
| | | | TTAATGCC | TCCC | |
| | | | (SEQ ID NO: 241) | (SEQ ID NO: 242) | |
| NM_005553.4 | 11 | rs2663, | CTGTGTGCTACCAGT | CCTGAGCCAAGGGCT | 134 |
| | | rs2664 | GCAAG | CTTT | |
| | | | (SEQ ID NO: 243) | (SEQ ID NO: 244) | |
| NM_005553.4 | 11 | rs10792769 | CAGCTGTGGAGGCTG | TAGAGCAGGAACAGG | 131 |
| | | | TGAC | CTGGA | |
| | | | (SEQ ID NO: 245) | (SEQ ID NO: 246) | |
| NM_005553.4 | 11 | rs2665 | TGGCTCAGGGCGTCT | AGTGCAGTTTGGCTC | 140 |
| | | | TTTTC | AGACATA | |
| | | | (SEQ ID NO: 247) | (SEQ ID NO: 248) | |
| NM_005734.5 | 11 | rs4755571 | AGAGTGTTTGCTGTA | TACTGCATGGTTTGC | 136 |
| | | | TAATTGGACTT | CCAAG | |
| | | | (SEQ ID NO: 249) | (SEQ ID NO: 250) | |
| NM_005734.5 | 11 | rs10600 | TGCAAGGTGTAGGAA | ATAGGAGTAAAAGCA | 137 |
| | | | GATTTGTTT | AAAACAAATAAACC | |
| | | | (SEQ ID NO: 251) | (SEQ ID NO: 252) | |
| NM_015065.3 | 11 | rs10749920 | CCAAGGTTGTCACCA | GGTCTTGTGGCCAGG | 139 |
| | | | GGAATTAAC | AGTTA | |
| | | | (SEQ ID NO: 253) | (SEQ ID NO: 254) | |
| NM_015065.3 | 11 | rs2640785 | CCTTTTTCTTCCCGG | CACAGGCATAGGAGG | 138 |
| | | | ATGAGC | TCCTG | |
| | | | (SEQ ID NO: 255) | (SEQ ID NO: 256) | |
| NM_015065.3 | 11 | rs4643041 | AGATGGCAGTGAAGA | TGGAAAATTAGATTA | 133 |
| | | | TAATCTGCT | GGTCGCGAAG | |
| | | | (SEQ ID NO: 257) | (SEQ ID NO: 258) | |
| NM_015065.3 | 11 | rs2640779 | CACTGTTCCCTGAGC | ACGTGTGTAGAAGGT | 139 |
| | | | CTACTC | TCTCTGT | |
| | | | (SEQ ID NO: 259) | (SEQ ID NO: 260) | |
| NM_015065.3 | 11 | rs2846412 | CCCACAGTCACTTTG | ACCAAGATATTGGGC | 137 |
| | | | CAGAAAAT | TGGCT | |
| | | | (SEQ ID NO: 261) | (SEQ ID NO: 262) | |
| NM_015065.3 | 11 | rs3741060 | TCTGTAAATGTTCTT | GTGCCTGGGATATTA | 139 |
| | | | AGCCTGCCT | GGGTTGT | |
| | | | (SEQ ID NO: 263) | (SEQ ID NO: 264) | |
| NM_015065.3 | 11 | rs2640738 | CACACACAGTAAAGA | TGCTGATCCTGTCCT | 138 |
| | | | AAATGACGAAAG | TCTCG | |
| | | | (SEQ ID NO: 265) | (SEQ ID NO: 266) | |
| NM_000424.4 | 12 | rs641615, | GCGAGCAGATCAAGA | CCAGGTTCTGCCTCA | 141 |
| | | rs641621 | CCCTC | CAGTC | |
| | | | (SEQ ID NO: 267) | (SEQ ID NO: 268) | |
| NM_000424.4 | 12 | rs1132948 | GCAGCTGGACAGCAT | CATCACAAACTCATT | 139 |
| | | | CGTG | CTCAGCAGT | |
| | | | (SEQ ID NO: 269) | (SEQ ID NO: 270) | |
| NM_000424.4 | 12 | rs4761924 | TGAGGTCAAGGCCCA | CTCATGCTTGGTGTT | 136 |
| | | | GTATG | GCGG | |
| | | | (SEQ ID NO: 271) | (SEQ ID NO: 272) | |
| NM_000424.4 | 12 | rs11549949, | CCTCGGTGGAGGTCT | CTGCTTGCACTGAAG | 138 |
| | | rs11549950 | TGG | CCAGA | |
| | | | (SEQ ID NO: 273) | (SEQ ID NO: 274) | |
| NM_000424.4 | 12 | rs6603 | GCTTCAAATCAGCCT | TCTGGAGGTAGTTAG | 137 |
| | | | TCAGGT | AACCAAAACA | |
| | | | (SEQ ID NO: 275) | (SEQ ID NO: 276) | |
| NM_002281.4 | 12 | rs79897879 | CAAACGTCCAGGAGG | CGGTAGCAGGAGATG | 134 |
| | | | ATCATG | CCA | |
| | | | (SEQ ID NO: 277) | (SEQ ID NO: 278) | |
| NM_002281.4 | 12 | rs2071588 | TGCTACCGCGGCCTC | TGACCGACACGGTGG | 142 |
| | | | ACC | TGATG | |
| | | | (SEQ ID NO: 279) | (SEQ ID NO: 280) | |
| NM_002281.4 | 12 | rs4761784, | GCCAAGTGCCAGAAC | GGCAGGCCATGTCCT | 139 |
| | | rs4761856 | TCCAAG | GCT | |
| | | | (SEQ ID NO: 281) | (SEQ ID NO: 282) | |
| NM_002281.4 | 12 | rs3187034, | CCTGGTACCGCAGCA | GGCACTTGGCATTCT | 141 |
| | | rs7300801 | AGTG | CCACC | |
| | | | (SEQ ID NO: 283) | (SEQ ID NO: 284) | |
| NM_002281.4 | 12 | rs3657 | AGCAGCTGCCGGAAA | GGGTCTTTCAAAGTG | 138 |
| | | | TGTTA | CAGGAG | |
| | | | (SEQ ID NO: 285) | (SEQ ID NO: 286) | |
| NM_002282.3 | 12 | rs2857663 | TCAAGTCCCTCAACA | TCCAGGTTACTCTGG | 127 |
| | | | GCAGA | CAGCA | |
| | | | (SEQ ID NO: 287) | (SEQ ID NO: 288) | |
| NM_002282.3 | 12 | rs2852464, | GAGATCCGCATTCTC | CTACGGGTGGCAATG | 131 |
| | | rs2857669 | CAATCC | TCATC | |
| | | | (SEQ ID NO: 289) | (SEQ ID NO: 290) | |
| NM_002 | 12 | rs2857670 | AGGTGTTGAAGCTGT | CAGTGCTCACCACCA | 140 |
| | | | GAATGT | GGTTC | |
| 282.3 | | | C | | |
| | | | (SEQ ID NO: 291) | (SEQ ID NO: 292) | |
| NM_002282.3 | 12 | rs2257286 | GCCGAGTCCTGGTAT | CATTCTCCACCTCGG | 139 |
| | | | CGCA | CTGT | |
| | | | (SEQ ID NO: 293) | (SEQ ID NO: 294) | |
| NM_002282.3 | 12 | rs2852456, | CTGGCTTTTCCCCTT | GGGCTGAAGGCTGAG | 139 |
| | | rs2857672 | GTGTTC | ACAG | |
| | | | (SEQ ID NO: 295) | (SEQ ID NO: 296) | |
| NM_002282.3 | 12 | rs3741715 | GCTGGCTACATCGAG | TCGAAGTGCTACTTC | 138 |
| | | | ACTCTG | TTCTTCATACT | |
| | | | (SEQ ID NO: 297) | (SEQ ID NO: 298) | |
| NM_002282.3 | 12 | rs2248473, | CCAAGTGCCAGAACT | ATCAGGCAGGCCATG | 142 |
| | | rs2852468 | CCAAG | TCTTG | |
| | | | (SEQ ID NO: 299) | (SEQ ID NO: 300) | |
| NM_002282.3 | 12 | rs2857671 | CCAGCTGAACACCAC | AGCGATGTGCTGCTG | 139 |
| | | | CTGT | TTTTC | |
| | | | (SEQ ID NO: 301) | (SEQ ID NO: 302) | |
| NM_002282.3 | 12 | rs2857667 | CACAGCAGAGAACGA | CGGATCTCCTCCTCG | 138 |
| | | | GTTTGTG | TACAG | |
| | | | (SEQ ID NO: 303) | (SEQ ID NO: 304) | |
| NM_002283.4 | 12 | rs7968466 | TGCACTGAATGGCAT | AGGATGCATCTCCCT | 139 |
| | | | GTGAATG | AGCATG | |
| | | | (SEQ ID NO: 305) | (SEQ ID NO: 306) | |
| NM_002283.4 | 12 | rs15667 | AGAAGACTCCATGGC | CCAGAAGGCAATGCA | 139 |
| | | | TGCTC | CCCATA | |
| | | | (SEQ ID NO: 307) | (SEQ ID NO: 308) | |
| NM_002283.4 | 12 | rs1059837 | TCCTACTTAGCTCCA | TGGCTGGATGGTTAG | 138 |
| | | | AAATACCTGT | GATGG | |
| | | | (SEQ ID NO: 309) | (SEQ ID NO: 310) | |
| NM_003482.4 | 12 | rs2241726 | TCAGCTGATGCCACC | AACGGTGACTCAGGG | 138 |
| | | | AGATC | TCACT | |
| | | | (SEQ ID NO: 311) | (SEQ ID NO: 312) | |
| NM_003482.4 | 12 | rs11168830 | TTGGTGAGCTCCCGA | GGCAAAGAGGCTAAA | 138 |
| | | | AAGAA | ATTGGC | |
| | | | (SEQ ID NO: 313) | (SEQ ID NO: 314) | |
| NM_003482.4 | 12 | rs10747559 | TTGCCCCATTGCATA | CTTGGCATGGAGCTC | 140 |
| | | | AGCCA | ACCT | |
| | | | (SEQ ID NO: 315) | (SEQ ID NO: 316) | |
| NM_003482.4 | 12 | rs3782357 | CACTAATCTCATGGC | CTGACCAGGGAGCTT | 139 |
| | | | AGAATATCGG | GGTG | |
| | | | (SEQ ID NO: 317) | (SEQ ID NO: 318) | |
| NM_003482.4 | 12 | rs3741622 | GGTCCATGACCCAGA | CATGATTCCAACCCC | 137 |
| | | | ACCTTC | AGGCA | |
| | | | (SEQ ID NO: 319) | (SEQ ID NO: 320) | |
| NM_021009.7 | 12 | rs1071862 | CGTGAAGACACTCAC | TTCCAGCTGCTTTCC | 142 |
| | | | TGGCA | GGCA | |
| | | | (SEQ ID NO: 321) | (SEQ ID NO: 322) | |
| NM_173086.5 | 12 | rs410562, | GTACAGTCCACCATC | TGGAACTGAAGCCAC | 124 |
| | | rs570463693 | TCCAGT | CTCCA | |
| | | | (SEQ ID NO: 323) | (SEQ ID NO: 324) | |
| NM_173086.5 | 12 | rs382367, | CAGGCTGCCATTGCT | TCAGCTCCTGGTACT | 139 |
| | | rs3894847 | GATG | CCTTCA | |
| | | | (SEQ ID NO: 325) | (SEQ ID NO: 326) | |
| NM_173086.5 | 12 | rs1053796, | TAAAGTGCTGCCTCC | AAGCATCCATACCCA | 137 |
| | | rs2567, | AGCTC | GCTCT | |
| | | rs2568 | (SEQ ID NO: 327) | (SEQ ID NO: 328) | |
| NM_173086.5 | 12 | rs583678 | ATACCTGCCCCATCT | GCCTCAGAGAGAACA | 137 |
| | | | GAGCA | ATTTTGGAG | |
| | | | (SEQ ID NO: 329) | (SEQ ID NO: 330) | |
| NM_173086.5 | 12 | rs372790, | TGAGGCTGAGCATTA | GGGTTCACAATACTT | 134 |
| | | rs432498 | TAAGAAAATGA | TTAACTTAGGGA | |
| | | | (SEQ ID NO: 331) | (SEQ ID NO: 332) | |
| NM_173086.5 | 12 | rs412533 | GGAGCTGATGAATGT | ACTGTACTACAGAGA | 125 |
| | | | CAAGCTG | CGTTGACTT | |
| | | | (SEQ ID NO: 333) | (SEQ ID NO: 334) | |
| NM_173086.5 | 12 | rs11608915 | TGCAAATTGACCCCG | AGAACCTTGTTCTGC | 124 |
| | | | CCA | TGCTCT | |
| | | | (SEQ ID NO: 335) | (SEQ ID NO: 336) | |
| NM_173086.5 | 12 | rs201833096, | GGTGCATGTGGAGGA | GCCACCAAAGCCATA | 141 |
| | | rs2885799, | GCTG | GCTG | |
| | | rs411107, | (SEQ ID NO: 337) | (SEQ ID NO: 338) | |
| | | rs419374, | | | |
| | | rs556681459 | | | |
| NM_178169.4 | 12 | rs8648 | TGCAATTTGGAAACC | TCTTTGGCCTTAGTG | 136 |
| | | | TACTGTATTGAA | GCCTTT | |
| | | | (SEQ ID NO: 339) | (SEQ ID NO: 340) | |
| NM_178169.4 | 12 | rs1797683 | GGGTTGACAGAGAAA | ACTCGTTTACAAGCA | 134 |
| | | | GCAAAGTG | ACTACCC | |
| | | | (SEQ ID NO: 341) | (SEQ ID NO: 342) | |
| NM_178169.4 | 12 | rs3741748 | CAGCTGCATAAGGAC | CTGCTGTAGCCGCTG | 142 |
| | | | TGCCC | CTCA | |
| | | | (SEQ ID NO: 343) | (SEQ ID NO: 344) | |
| NM_178169.4 | 12 | rs77813527 | GCCGAGGACTTCTTC | TTTCTCTTTGATCTC | 138 |
| | | | TTCACC | CTCTTTGCT | |
| | | | (SEQ ID NO: 345) | (SEQ ID NO: 346) | |
| NM_014572.3 | 13 | rs2770928 | CCGCAGAGCCTGCTC | CCTCCAGGCCCGGCT | 133 |
| | | | ACTC | TCT | |
| | | | (SEQ ID NO: 347) | (SEQ ID NO: 348) | |
| NM_014572.3 | 13 | rs7317471 | AGACTTTTCCTGCCA | TTGGCATCCAGGGAA | 139 |
| | | | CGACTT | GTGTC | |
| | | | (SEQ ID NO: 349) | (SEQ ID NO: 350) | |
| NM_014572.3 | 13 | rs558614 | GCCGGCCTCGCTTTC | GGCTGTCGCTGGCGA | 130 |
| | | | CCA | ACA | |
| | | | (SEQ ID NO: 351) | (SEQ ID NO: 352) | |
| NM_014832.5 | 13 | rs1062087 | GATTGGACAGGAACG | GCTCCTCAGTTCTTT | 137 |
| | | | AGCTG | TGATTTCTTTCT | |
| | | | (SEQ ID NO: 353) | (SEQ ID NO: 354) | |
| NM_014832.5 | 13 | rs557337 | ATCCGGACCCTGGAA | TCTTGGCTTTGTTGT | 139 |
| | | | CAAGA | TAGGGTTG | |
| | | | (SEQ ID NO: 355) | (SEQ ID NO: 356) | |
| NM_014832.5 | 13 | rs7327548 | CCTGGTGAGGCGAGA | ACAGCCGGAACCGCT | 133 |
| | | | GTTATG | TATC | |
| | | | (SEQ ID NO: 357) | (SEQ ID NO: 358) | |
| NM_014832.5 | 13 | rs77685055 | GAGAGGTGATCCTGG | GATATGCTGCGCCTT | 140 |
| | | | TGCTC | GTGC | |
| | | | (SEQ ID NO: 359) | (SEQ ID NO: 360) | |
| NM_014832.5 | 13 | rs2297208 | GCCTAATAAACAACA | TGGCCCAAGCTGTAC | 136 |
| | | | GCCTCCTG | TGAAA | |
| | | | (SEQ ID NO: 361) | (SEQ ID NO: 362) | |
| NM_001969.5 | 14 | rs12588499 | CCTGAGGCATAGGTA | CTGGTAGGTCAAGCT | 140 |
| | | | CTGTTGT | GTAGCA | |
| | | | (SEQ ID NO: 363) | (SEQ ID NO: 364) | |
| NM_001969.5 | 14 | rs8020483 | GGTTGCTTTCTTGCC | CCAAAATAGAGGCTC | 138 |
| | | | AAAATAAGT | TGACTTAGAAG | |
| | | | (SEQ ID NO: 365) | (SEQ ID NO: 366) | |
| NM_001969.5 | 14 | rs4246 | GGTCTAGCAATACAC | TCTGAGATTCAAAGA | 119 |
| | | | TGTTTACAAGAG | GCTACATTTTTG | |
| | | | (SEQ ID NO: 367) | (SEQ ID NO: 368) | |
| NM_001969.5 | 14 | rs12896 | GCGCTGACGGGTGTG | AAGTGAGGCGCGATA | 141 |
| | | | GAC | TAAACCC | |
| | | | (SEQ ID NO: 369) | (SEQ ID NO: 370) | |
| NM_001969.5 | 14 | rs1883 | TGCATTGCACGTTGG | CTTCCTTGTTTCTCC | 138 |
| | | | ATGAG | CAAATAAAATCA | |
| | | | (SEQ ID NO: 371) | (SEQ ID NO: 372) | |
| NM_001969.5 | 14 | rs7143775 | GCTTAATAGGGAACA | GGACCCACACTAAAA | 138 |
| | | | TACATACAGCTATG | ATTTTTAAGTCAAT | |
| | | | (SEQ ID NO: 373) | (SEQ ID NO: 374) | |
| NM_001969.5 | 14 | rs10289 | TCCCAGGCCTTACTG | ATTTCCCCTACTGCC | 139 |
| | | | TAAAACTAG | CCAGC | |
| | | | (SEQ ID NO: 375) | (SEQ ID NO: 376) | |
| NM_004050.5 | 14 | rs2231301 | CTCTGGTGGCAGACT | AAGCGGGTCTCGAAC | 136 |
| | | | TTGTAG | TCATC | |
| | | | (SEQ ID NO: 377) | (SEQ ID NO: 378) | |
| NM_004050.5 | 14 | rs1950252 | TTAACAGTGGGCTGC | TGCCCCTAGTTCTTG | 138 |
| | | | TTGGA | CCATT | |
| | | | (SEQ ID NO: 379) | (SEQ ID NO: 380) | |
| NM_004050.5 | 14 | rs10149339 | GTGCTTCCTTCCCCA | AGCAGGGCATTTTGG | 138 |
| | | | GGTAT | TAACCT | |
| | | | (SEQ ID NO: 381) | (SEQ ID NO: 382) | |
| NM_005348.4 | 14 | rs4947 | CAGAGCCCTTCTATT | CAGATATTCAGGGAT | 136 |
| | | | TGTCCCA | TAGCTCCTCA | |
| | | | (SEQ ID NO: 383) | (SEQ ID NO: 384) | |
| NM_005348.4 | 14 | rs1059623 | CTAGCTGCTCCAAGG | CACTAAGAGAACACA | 138 |
| | | | GTTGA | TGTAACTCATGG | |
| | | | (SEQ ID NO: 385) | (SEQ ID NO: 386) | |
| NM_032866.5 | 15 | rs11856, | TTGGCAACGCATGCG | TGTTCCAGGTAATCC | 137 |
| | | rs8784 | GTATA | ATGGCT | |
| | | | (SEQ ID NO: 387) | (SEQ ID NO: 388) | |
| NM_032866.5 | 15 | rs12439292 | TGGAACAAAGTGCAG | AGCTAAAGAAAATAC | 139 |
| | | | ATTAGTGC | TCTTGATCGGG | |
| | | | (SEQ ID NO: 389) | (SEQ ID NO: 390) | |
| NM_032866.5 | 15 | rs3742966, | GGTAAACAAGTCACT | TCAGTGACTGGGAAC | 138 |
| | | rs937248 | GGCACAG | AGGTC | |
| | | | (SEQ ID NO: 391) | (SEQ ID NO: 392) | |
| NM_032866.5 | 15 | rs1620402 | AGAAGAGAGAAACAA | CCTCTCCAGGGAAAT | 139 |
| | | | CTCAGATTTGC | CTTGTCG | |
| | | | (SEQ ID NO: 393) | (SEQ ID NO: 394) | |
| NM_032866.5 | 15 | rs16977594 | GGCAGCTCAACTCCA | TGGAGAATGTGTAGC | 138 |
| | | | TGAAGA | TCACAGG | |
| | | | (SEQ ID NO: 395) | (SEQ ID NO: 396) | |
| NM_032866.5 | 15 | rs2131730, | GGAGCATCTGTCTGC | TCAGGCATCCCAACA | 139 |
| | | rs2131731 | CACT | TTTTAAACT | |
| | | | (SEQ ID NO: 397) | (SEQ ID NO: 398) | |
| NM_032866.5 | 15 | rs7166305 | TCTCACCTCTGCCTG | GCCTGTTTAATTCAC | 140 |
| | | | CTTATG | TGCTGTATTCT | |
| | | | (SEQ ID NO: 399) | (SEQ ID NO: 400) | |
| NM_032866.5 | 15 | rs2934418 | CTTGTTGCCGAGGCT | GAGTGGTGGTGGTCA | 126 |
| | | | GGA | TCTGTAA | |
| | | | (SEQ ID NO: 401) | (SEQ ID NO: 402) | |
| NM_032866.5 | 15 | rs17820431, | AGTTCTTTCCAGTAG | TGTAGGATAGCGCAA | 140 |
| | | rs17820437 | CCATGGG | TTGTCAAAA | |
| | | | (SEQ ID NO: 403) | (SEQ ID NO: 404) | |
| NM_032866.5 | 15 | rs1280396 | GGTGCACAGAGTAAA | TGAGTATTTGAGGCC | 140 |
| | | | AAGGAGGA | GAAGGAA | |
| | | | (SEQ ID NO: 405) | (SEQ ID NO: 406) | |
| NM_032866.5 | 15 | rs7182648 | CCTTTGCAAAGCTGC | GCGCACGGATCACTG | 126 |
| | | | AGGGA | TACTT | |
| | | | (SEQ ID NO: 407) | (SEQ ID NO: 408) | |
| NM_032866.5 | 15 | rs1666573 | ACTTAATAGCACTGT | GCATGAACTTTACAA | 136 |
| | | | GCAGGGA | ACCAGCCA | |
| | | | (SEQ ID NO: 409) | (SEQ ID NO: 410) | |
| NM_032866.5 | 15 | rs3742963, | TGTCCCCAACCATCC | AGACTCCCACCCTTA | 139 |
| | | rs3742964 | CAATAG | AACTGG | |
| | | | (SEQ ID NO: 411) | (SEQ ID NO: 412) | |
| NM_183376.3 | 15 | rs1062702 | ACAATGCAAAACTGT | AGACAACACTTAAAT | 131 |
| | | | TAGACCAGTA | ACACATTACCTGTT | |
| | | | (SEQ ID NO: 413) | (SEQ ID NO: 414) | |
| NM_183376.3 | 15 | rs112100339, | GCTGGAGGCGTCCGA | GGTACTCCACCTCCG | 143 |
| | | rs12101554 | GCC | AGAAGACAG | |
| | | | (SEQ ID NO: 415) | (SEQ ID NO: 416) | |
| NM_183376.3 | 15 | rs3803385 | TGAACGTATTTCAGA | TCTATTGCCTTCAGT | 133 |
| | | | AATCACTGTGTT | CTTGACG | |
| | | | (SEQ ID NO: 417) | (SEQ ID NO: 418) | |
| NM_183376.3 | 15 | rs3169159, | TGGTCATATGAGGTC | CCTCTTTGAACCGAC | 136 |
| | | rs3169160 | CTAATGAAGT | AAGCTTT | |
| | | | (SEQ ID NO: 419) | (SEQ ID NO: 420) | |
| NM_183 | 15 | rs1043372 | CCATTTGATCAGGGA | TGTGGGAATTTAAAT | 136 |
| | | | ATTTTCT | AGCGG | |
| 376.3 | | | ACAC | CTT | |
| | | | (SEQ ID NO: 421) | (SEQ ID NO: 422) | |
| NM_183376.3 | 15 | rs4965046 | CCTCTGCCGACCTCA | GAATAGCAGCCCTTG | 139 |
| | | | GGG | CGCT | |
| | | | (SEQ ID NO: 423) | (SEQ ID NO: 424) | |
| NM_183376.3 | 15 | rs1043374 | TCATGAAGTGTCCCT | ATCTTCAACCTACTG | 127 |
| | | | GTGAAATTG | CTTACAGTC | |
| | | | (SEQ ID NO: 425) | (SEQ ID NO: 426) | |
| NM_183376.3 | 15 | rs1043382 | TTGAAACAGAAAATA | TGCATCAAAATGAAA | 129 |
| | | | CCTGGGAATG | ATTCCAAGGC | |
| | | | (SEQ ID NO: 427) | (SEQ ID NO: 428) | |
| NM_183376.3 | 15 | rs2130882 | GGATATGAGCTGGTT | GCACACTTCTCCCTC | 139 |
| | | | GACACTGA | ACAGT | |
| | | | (SEQ ID NO: 429) | (SEQ ID NO: 430) | |
| NM_183376.3 | 15 | rs9652498 | CCCCTTTAAGTGCTT | GCCTTTTATTTGGGA | 139 |
| | | | TAACAGGAT | CTGTGTAGTTATC | |
| | | | (SEQ ID NO: 431) | (SEQ ID NO: 432) | |
| NM_002705.5 | 16 | rs2037912 | CGGAGCTGGAATTCC | TCGCTGCCATGTTGA | 138 |
| | | | ATAACTC | TTTCC | |
| | | | (SEQ ID NO: 433) | (SEQ ID NO: 434) | |
| NM_002705.5 | 16 | rs1049206 | GAAAGATATGGGCCT | TACTTTCGCTCCTGC | 139 |
| | | | TGGAGGA | TCCAC | |
| | | | (SEQ ID NO: 435) | (SEQ ID NO: 436) | |
| NM_002705.5 | 16 | rs1049207, | GGAAAGAGGAGGAGC | AGCTCCACATCGATG | 124 |
| | | rs1049208 | TCTCG | CTCTC | |
| | | | (SEQ ID NO: 437) | (SEQ ID NO: 438) | |
| NM_002705.5 | 16 | rs2075639 | CAGGAGTACGTGGTC | CAGGAGGAGCACCTC | 135 |
| | | | AAGGAG | TGC | |
| | | | (SEQ ID NO: 439) | (SEQ ID NO: 440) | |
| NM_002705.5 | 16 | rs1049212 | ACACCAGACAGTGAC | GTCTCATGAGCAGAG | 138 |
| | | | ACTGC | TAACTACTGA | |
| | | | (SEQ ID NO: 441) | (SEQ ID NO: 442) | |
| NM_002705.5 | 16 | rs1049205 | TGCGGATTGAACCTG | TGCAGGAGGTGCTCG | 136 |
| | | | AGAAGA | TATTT | |
| | | | (SEQ ID NO: 443) | (SEQ ID NO: 444) | |
| NM_002705.5 | 16 | rs1876363 | ACAGCTACACCCTGC | CCAGGCTGTCAGCCA | 138 |
| | | | AGAAG | GAG | |
| | | | (SEQ ID NO: 445) | (SEQ ID NO: 446) | |
| NM_002705.5 | 16 | rs7214 | TGATTATTCACCACG | ACCGTACAAGCAGAC | 126 |
| | | | CCAATATCTG | TCCAG | |
| | | | (SEQ ID NO: 447) | (SEQ ID NO: 448) | |
| NM_001123387.1 | 17 | rs6503578, | GCCTCAGACACCACC | CGGCACACGGTGGTC | 140 |
| | | rs9635728 | ATGAC | TGG | |
| | | | (SEQ ID NO: 449) | (SEQ ID NO: 450) | |
| NM_001123387.1 | 17 | rs6503577 | CACACTCCCATTTCT | GAAATTGCTTCTTTT | 137 |
| | | | CTACTTTAACA | ACAGACTTCAGA | |
| | | | (SEQ ID NO: 451) | (SEQ ID NO: 452) | |
| NM_001277332.1 | 17 | rs4890107, | TTGCTGTCAGCCTAC | CAAGTTGGGTGGCAG | 132 |
| | | rs78283750 | CTGCT | CAAG | |
| | | | (SEQ ID NO: 453) | (SEQ ID NO: 454) | |
| NM_001386014.1 | 17 | rs1051602 | GGGTCGAGCCTGTTT | TCCTCCTTGCATTCC | 139 |
| | | | GTTTC | AGAGAAT | |
| | | | (SEQ ID NO: 455) | (SEQ ID NO: 456) | |
| NM_001386014.1 | 17 | rs2239710 | TGAACCAGGTCCTGA | AGGACTGCAGCTGCT | 141 |
| | | | ACGAG | CTGA | |
| | | | (SEQ ID NO: 457) | (SEQ ID NO: 458) | |
| NM_001386014.1 | 17 | rs2071599 | CAACGCCCTGGAGAT | TCTGCCAGCTGAGAC | 144 |
| | | | CGAG | TCCA | |
| | | | (SEQ ID NO: 459) | (SEQ ID NO: 460) | |
| NM_002275.4 | 17 | rs1050784 | AATGCTGACCTGGAG | ATGGTGGTGGCCATG | 128 |
| | | | GTGAA | ATCTT | |
| | | | (SEQ ID NO: 461) | (SEQ ID NO: 462) | |
| NM_002275.4 | 17 | rs2305556 | GACATAAAGACACGG | TGTGGAAATTGCTGC | 133 |
| | | | CTGGAG | TGCTA | |
| | | | (SEQ ID NO: 463) | (SEQ ID NO: 464) | |
| NM_002277.3 | 17 | rs1047449 | ACATCATTTGCATGG | TCTGGGTGAGCATAG | 139 |
| | | | CTGGAG | GAAGGA | |
| | | | (SEQ ID NO: 465) | (SEQ ID NO: 466) | |
| NM_002277.3 | 17 | rs1047448 | GTGGGCCCTGCAATT | TGATGTTTTCAGGCC | 123 |
| | | | CCTT | CCTTTTG | |
| | | | (SEQ ID NO: 467) | (SEQ ID NO: 468) | |
| NM_002278.3 | 17 | rs72830046 | CCTTAACATCGAGGT | AGCTCCTCCATCTGC | 138 |
| | | | GGACG | ATATTGAA | |
| | | | (SEQ ID NO: 469) | (SEQ ID NO: 470) | |
| NM_002278.3 | 17 | rs3744786 | CAGCTGCCTCTCCAA | GTCGTTAAGGAACTG | 139 |
| | | | AACCTA | CATGGTT | |
| | | | (SEQ ID NO: 471) | (SEQ ID NO: 472) | |
| NM_002278.3 | 17 | rs12948056 | AGGAGCTTAACCAAC | AGTCCCTCAGGCTGT | 129 |
| | | | AGGTGG | GCT | |
| | | | (SEQ ID NO: 473) | (SEQ ID NO: 474) | |
| NM_002278.3 | 17 | rs2604953 | CACCACCTGTGTGCC | GCCCTCCAGGATCCA | 128 |
| | | | CTC | CTGG | |
| | | | (SEQ ID NO: 475) | (SEQ ID NO: 476) | |
| NM_002278.3 | 17 | rs2071563, | GTACCAGGTGCTGCT | CAGGTGGTGCAGGAA | 126 |
| | | rs2604954, | GGAC | GGAG | |
| | | rs2604955 | (SEQ ID NO: 477) | (SEQ ID NO: 478) | |
| NM_002278.3 | 17 | rs2071561 | CGCAGGATCCTGGAT | GCATCGAAGGGAACC | 123 |
| | | | GATCTC | GACTT | |
| | | | (SEQ ID NO: 479) | (SEQ ID NO: 480) | |
| NM_002278.3 | 17 | rs11078993, | GGGACTCCCTGGAAA | TTCTGCCGCTCCAGG | 130 |
| | | rs2857257 | ACACG | TCAG | |
| | | | (SEQ ID NO: 481) | (SEQ ID NO: 482) | |
| NM_002279.5 | 17 | rs71373411 | GTATCCAGCTCGGAG | CGGGCCTCGCTCTCT | 143 |
| | | | CAGC | GTC | |
| | | | (SEQ ID NO: 483) | (SEQ ID NO: 484) | |
| NM_002280.6 | 17 | rs743686 | GCTTCTCTTCTGGGT | GCAGGCAGAGAAACT | 131 |
| | | | CTCTCA | TCTGG | |
| | | | (SEQ ID NO: 485) | (SEQ ID NO: 486) | |
| NM_002280.6 | 17 | rs7216017 | CTGAACCAGCAGGTG | CAGGGTGGATTCCAA | 141 |
| | | | GTGTC | AGCATC | |
| | | | (SEQ ID NO: 487) | (SEQ ID NO: 488) | |
| NM_002280.6 | 17 | rs12451652, | TCATGCCTTCCCTGT | AGACAATAGCCATGG | 139 |
| | | rs2071601, | CTTCC | CCATCT | |
| | | rs2239711 | (SEQ ID NO: 489) | (SEQ ID NO: 490) | |
| NM_004138.4 | 17 | rs12937519 | GCAGGTGGTATCCAG | TCTCTGTCAGCGTGT | 140 |
| | | | CTCG | TTTCCA | |
| | | | (SEQ ID NO: 491) | (SEQ ID NO: 492) | |
| NM_004138.4 | 17 | rs16966703 | GCCACAACCAATGCA | TCTTCTCCTCCTGGT | 124 |
| | | | TGTGAC | TGTGG | |
| | | | (SEQ ID NO: 493) | (SEQ ID NO: 494) | |
| NM_016113.5 | 17 | rs1129235, | GAGGGCTCCACAGGT | TGTGCACTGGGCATT | 138 |
| | | rs8121 | AAGAC | TACCA | |
| | | | (SEQ ID NO: 495) | (SEQ ID NO: 496) | |
| NM_016113.5 | 17 | rs14039 | GGAGATGGAGAATGG | TCCCATGAAGCCCAG | 138 |
| | | | CTATTGGT | TTCAC | |
| | | | (SEQ ID NO: 497) | (SEQ ID NO: 498) | |
| NM_016113.5 | 17 | rs77272400 | CCTGTCCTGACAGGG | CGGAACCTCCCTCCC | 130 |
| | | | GAGAG | CAG | |
| | | | (SEQ ID NO: 499) | (SEQ ID NO: 500) | |
| NM_025099.6 | 17 | rs3826543 | TCCGGTCATCCACTT | ACGATATGGCAAGAG | 139 |
| | | | ATGTGC | GCAGT | |
| | | | (SEQ ID NO: 501) | (SEQ ID NO: 502) | |
| NM_025099.6 | 17 | rs74506939 | TGCTGCTGAAACCAG | AGAATACCGGACAAG | 137 |
| | | | AATGTG | ACTTTCTG | |
| | | | (SEQ ID NO: 503) | (SEQ ID NO: 504) | |
| NM_030967.3 | 17 | rs3815561 | GAGCTGCACACCCCC | GCAGAAGGAGGTTTT | 141 |
| | | | ATC | CAACAAGTG | |
| | | | (SEQ ID NO: 505) | (SEQ ID NO: 506) | |
| NM_030967.3 | 17 | rs112542604, | TCCAAGCAACCCAAC | GAGCTGGTCTCACAG | 138 |
| | | rs141856614 | CTTCA | CAGCT | |
| | | | (SEQ ID NO: 507) | (SEQ ID NO: 508) | |
| NM_031959.3 | 17 | rs3829598, | AGCTAACCAGACGCC | CTGTGTGTGGGCAGG | 140 |
| | | rs9890989, | CATTG | TGCT | |
| | | rs9897046 | (SEQ ID NO: 509) | (SEQ ID NO: 510) | |
| NM_031959.3 | 17 | rs3813048, | CACACACAGTTTGGT | TGGTGAGGTTGAGGG | 141 |
| | | rs3813050 | TACTGGA | TCTCC | |
| | | | (SEQ ID NO: 511) | (SEQ ID NO: 512) | |
| NM_031959.3 | 17 | rs3813047 | TACTTTGCTCAGTGC | TCCCCCATTAAAACC | 136 |
| | | | CCGAC | AAAGGTAAG | |
| | | | (SEQ ID NO: 513) | (SEQ ID NO: 514) | |
| NM_031960.3 | 17 | rs375280428 | CTGCTGCAGACCCCA | GCAGCAGCTAGGGTG | 142 |
| | | | GTG | GCA | |
| | | | (SEQ ID NO: 515) | (SEQ ID NO: 516) | |
| NM_031960.3 | 17 | rs72625995, | CCCTGCTGCTGCCTG | GCCAGGGCAGTGGGC | 135 |
| | | rs77163010 | CGT | TCA | |
| | | | (SEQ ID NO: 517) | (SEQ ID NO: 518) | |
| NM_031960.3 | 17 | rs7212517, | CCTGGCTCATCTCTC | GGGATGGTGATGGGC | 137 |
| | | rs72625994 | CCTTC | TCATT | |
| | | | (SEQ ID NO: 519) | (SEQ ID NO: 520) | |
| NM_031960.3 | 17 | rs147906218, | GCCGCCCCAGCTGCT | GGGCGGCAGCAGCTG | 142 |
| | | rs76327324, | GTG | GATT | |
| | | rs80168553 | (SEQ ID NO: 521) | (SEQ ID NO: 522) | |
| NM_031960.3 | 17 | rs6503593, | ACTCCCTCCCTTGCT | GGAAGGATATTCTGT | 139 |
| | | rs8067655 | TTCTTTT | AGGCTCAAAATG | |
| | | | (SEQ ID NO: 523) | (SEQ ID NO: 524) | |
| NM_033185.3 | 17 | rs8072775 | GCTGTTCGATTCCTT | GCTATGCCAGAGATG | 140 |
| | | | CATATTAAAGTGT | TAAAATTTATTAGGA | |
| | | | (SEQ ID NO: 525) | TA | |
| | | | | (SEQ ID NO: 526) | |
| NM_033185.3 | 17 | rs1989365, | CCTTATCCTTCTTTT | CTCCACAGCGGCAGG | 139 |
| | | rs2010201 | CAATCAAACCC | ATT | |
| | | | (SEQ ID NO: 527) | (SEQ ID NO: 528) | |
| NM_033185.3 | 17 | rs12452295 | CACACACAGTTTGGT | TGGTGAGGTTGAGGG | 141 |
| | | | TACTGGA | TCTCC | |
| | | | (SEQ ID NO: 529) | (SEQ ID NO: 530) | |
| NM_004823.3 | 19 | rs4386247 | AATACTTGCCCAAGG | AGGTAAGGAACCGAG | 140 |
| | | | TCACAC | AAGGTTG | |
| | | | (SEQ ID NO: 531) | (SEQ ID NO: 532) | |
| NM_004823.3 | 19 | rs61160177 | AGTTTGAGGCTGCAG | CTTCCATCTTTCCCT | 139 |
| | | | TGAGTT | TTCTTTCTTTC | |
| | | | (SEQ ID NO: 533) | (SEQ ID NO: 534) | |
| NM_004823.3 | 19 | rs931750 | GACTTTAATGGAGGA | CTTAGTTCGATGTCC | 137 |
| | | | GGTGAGGT | CTTCCTCT | |
| | | | (SEQ ID NO: 535) | (SEQ ID NO: 536) | |
| NM_004823.3 | 19 | rs3745952 | TCCACTGGGCACCAA | GAACATGAAAGAGAA | 138 |
| | | | CATAA | TGAGAAACAGAG | |
| | | | (SEQ ID NO: 537) | (SEQ ID NO: 538) | |
| NM_004823.3 | 19 | rs931749 | GCAGGAAGAAGTTGT | GTCCTATGTCTGTTT | 134 |
| | | | ACCTGACT | CCTGCCT | |
| | | | (SEQ ID NO: 539) | (SEQ ID NO: 540) | |
| NM_004823.3 | 19 | rs8106900 | GGGCGAAACAGAGAC | CTGGGCTCAGGCAAT | 130 |
| | | | TCTGTC | CCTC | |
| | | | (SEQ ID NO: 541) | (SEQ ID NO: 542) | |
| NM_004823.3 | 19 | rs6508786 | GCAGTGAGCTGTGAT | GAATCGTGCCTGGCA | 139 |
| | | | TGCATC | CATAG | |
| | | | (SEQ ID NO: 543) | (SEQ ID NO: 544) | |
| NM_004823.3 | 19 | rs34989303 | CTGGTCACAGTCTAC | CATCCGCATTGAAAC | 139 |
| | | | CTCTTCC | TGGCA | |
| | | | (SEQ ID NO: 545) | (SEQ ID NO: 546) | |
| NM_004823.3 | 19 | rs73041302 | CCATGACACCATTGT | GGAAACAAAATCAGA | 135 |
| | | | AAGAGCTG | GAGGTAGTGT | |
| | | | (SEQ ID NO: 547) | (SEQ ID NO: 548) | |
| NM_004823.3 | 19 | rs77862444 | ATGAAGAGGGAGACG | CACTCTGTGGGACAT | 137 |
| | | | ATGGG | TCCCTA | |
| | | | (SEQ ID NO: 549) | (SEQ ID NO: 550) | |
| NM_004823.3 | 19 | rs3745951 | CACTTGGTGGCCCTG | CCGATGGTGGACAGA | 134 |
| | | | TTG | GAGATA | |
| | | | (SEQ ID NO: 551) | (SEQ ID NO: 552) | |
| NM_004823.3 | 19 | rs118185142, | GAAGAAAAGCAAGTC | CATGTCCAGGCTGGT | 137 |
| | | rs12609247 | CTTTTTGGT | CTCC | |
| | | | (SEQ ID NO: 553) | (SEQ ID NO: 554) | |
| NM_014330.5 | 19 | rs500079 | GAGCAGCTTGCTCGG | GGCTAAAGGTGGGTT | 129 |
| | | | GATC | CCTGA | |
| | | | (SEQ ID NO: 555) | (SEQ ID NO: 556) | |
| NM_014330.5 | 19 | rs611251 | AGAGGAGGGAGTTAA | TGGATCCTGGAGACA | 137 |
| | | | CAAGTTCTC | AGGCA | |
| | | | (SEQ ID NO: 557) | (SEQ ID NO: 558) | |
| NM_014330.5 | 19 | rs557806, | CCAGGAAGACCTCCG | GCAGCTTCTCCTTTC | 127 |
| | | rs610308 | TGTC | CCAGT | |
| | | | (SEQ ID NO: 559) | (SEQ ID NO: 560) | |
| NM_014330.5 | 19 | rs564196 | TCTTCCTCCTGTCCC | CCTCCAGGCCAGCTT | 129 |
| | | | CAGTG | CTAC | |
| | | | (SEQ ID NO: 561) | (SEQ ID NO: 562) | |
| NM_014330.5 | 19 | rs11541192, | CAAGTCCTGGTGGTG | CCAATACACCCAGGC | 139 |
| | | rs556052 | CCAA | CTTCAG | |
| | | | (SEQ ID NO: 563) | (SEQ ID NO: 564) | |
| NM_014330.5 | 19 | rs626974 | CCTGTGATCGCTTCT | AGGAAGAAAGGGTGG | 138 |
| | | | GGCA | GCATC | |
| | | | (SEQ ID NO: 565) | (SEQ ID NO: 566) | |
| NM_014330.5 | 19 | rs524, | CCTTCCTCCTCTGTC | GCAAACCAGTTGGTC | 138 |
| | | rs527 | CCTTC | TCAGC | |
| | | | (SEQ ID NO: 567) | (SEQ ID NO: 568) | |
| NM_144691.4 | 19 | rs73038948 | AAAGGAGGATGGCGA | CCTTGGAAGGTGTGG | 138 |
| | | | GTTCT | ACGTG | |
| | | | (SEQ ID NO: 569) | (SEQ ID NO: 570) | |
| NM_144691.4 | 19 | rs936524 | CTGGGTAACTGCTGG | AACTGCCAGAGCTGG | 134 |
| | | | TTCCTT | AAGTG | |
| | | | (SEQ ID NO: 571) | (SEQ ID NO: 572) | |
| NM_144691.4 | 19 | rs1060186 | CTGCGTACTCCTCAC | TCTCCTGTGTCTGTC | 138 |
| | | | TCAGA | CTCCA | |
| | | | (SEQ ID NO: 573) | (SEQ ID NO: 574) | |
| NM_144691.4 | 19 | rs76443569 | TTAACGAGGGAAGGC | TGGATGCCATCTGGA | 132 |
| | | | TCCTC | CACTG | |
| | | | (SEQ ID NO: 575) | (SEQ ID NO: 576) | |
| NM_144691.4 | 19 | rs12983010 | TGTTCCTGGGCTTCA | TTTTCACCAGCAGGG | 141 |
| | | | CCAAG | CATCG | |
| | | | (SEQ ID NO: 577) | (SEQ ID NO: 578) | |
| NM_144691.4 | 19 | rs4801861 | CAGCTGCTGCAGTGT | AGCTCGTAGGAGTTC | 140 |
| | | | TTCG | ATGGTTC | |
| | | | (SEQ ID NO: 579) | (SEQ ID NO: 580) | |
| NM_005560.6 | 20 | rs7262393 | CCTCTGTGACGAGCT | CTGAGCAGTTACACT | 128 |
| | | | CACG | CCTCACA | |
| | | | (SEQ ID NO: 581) | (SEQ ID NO: 582) | |
| NM_005560.6 | 20 | rs6142734 | GCTATGCCCTAGTGG | GCACAGGGTGCTGTA | 131 |
| | | | TGGAG | CAGG | |
| | | | (SEQ ID NO: 583) | (SEQ ID NO: 584) | |
| NM_005560.6 | 20 | rs483876, | CTACCAGCCCTGGCA | GGGCACGATGCGTGA | 130 |
| | | rs624313 | GTTC | GTA | |
| | | | (SEQ ID NO: 585) | (SEQ ID NO: 586) | |
| NM_005560.6 | 20 | rs6089732 | GCTGCAGCACAGAGA | TTCAAAGCCTCTCGC | 140 |
| | | | TTGC | AGGTC | |
| | | | (SEQ ID NO: 587) | (SEQ ID NO: 588) | |
| NM_005560.6 | 20 | rs8124907 | CGGCTACTTTCACTT | CGGCACCGGTCACAA | 132 |
| | | | CCCTCTC | TGA | |
| | | | (SEQ ID NO: 589) | (SEQ ID NO: 590) | |
| NM_005560.6 | 20 | rs2427289 | CACTGCTCTGCTGAA | GAGCCAGCTTCGCAG | 140 |
| | | | GGCT | TAGG | |
| | | | (SEQ ID NO: 591) | (SEQ ID NO: 592) | |
| NM_005560.6 | 20 | rs6062223 | CTGCGAGGGACCACT | CCCTCCAGCTGAAGT | 129 |
| | | | ACCT | TCTCG | |
| | | | (SEQ ID NO: 593) | (SEQ ID NO: 594) | |
| NM_005560.6 | 20 | rs2427284 | CCTGGAGCAGCTGCA | CACAGGCACAGCTCC | 132 |
| | | | GATC | ACATT | |
| | | | (SEQ ID NO: 595) | (SEQ ID NO: 596) | |
| NM_005560.6 | 20 | rs2297587 | GCATCAATGCCAGCT | TCTGCTGCTCCAGCA | 138 |
| | | | CCATG | CCT | |
| | | | (SEQ ID NO: 597) | (SEQ ID NO: 598) | |
| NM_005560.6 | 20 | rs2151513 | GGGATACCCAGGACC | TCCGGGCTGAACTCC | 127 |
| | | | ACCTG | TCAAT | |
| | | | (SEQ ID NO: 599) | (SEQ ID NO: 600) | |
| NM_005560.6 | 20 | rs944895 | GGAGCAGGACAATGA | CCAGGGCCTTGATGC | 140 |
| | | | TCTGGAG | CTTT | |
| | | | (SEQ ID NO: 601) | (SEQ ID NO: 602) | |
| NM_005560.6 | 20 | rs11204472 | TCGTCCTCCAATGAC | GAAGTTGGGTTTGCA | 138 |
| | | | ACCAG | CAGACA | |
| | | | (SEQ ID NO: 603) | (SEQ ID NO: 604) | |
| NM_005560.6 | 20 | rs3737137, | CAACTGCACCCGGTG | CGCAGCCATCGAAAC | 140 |
| | | rs6143021 | CGA | CAAAATG | |
| | | | (SEQ ID NO: 605) | (SEQ ID NO: 606) | |
| NM_005560.6 | 20 | rs3810548 | ATCATCCTCAGGGAC | GCTCTGCATCAGGGT | 136 |
| | | | TGCCA | CCAC | |
| | | | (SEQ ID NO: 607) | (SEQ ID NO: 608) | |
| NM_005560.6 | 20 | rs11698080, | CTGGCGTCTGTGTGG | TGTTGGAAGGCACTG | 138 |
| | | rs2427283 | ACTG | AGAGG | |
| | | | (SEQ ID NO: 609) | (SEQ ID NO: 610) | |
| NM_005560.6 | 20 | rs2427285 | CATTCCTGGAGCCGG | TGGCCAGCACCATCA | 132 |
| | | | CATAC | TGAG | |
| | | | (SEQ ID NO: 611) | (SEQ ID NO: 612) | |
| NM_005560.6 | 20 | rs4925229 | AGAGCCTGGATGGCA | CGAGTCGAGAGGGTG | 125 |
| | | | CCTAT | GTTG | |
| | | | (SEQ ID NO: 613) | (SEQ ID NO: 614) | |
| NM_005560.6 | 20 | rs13042941, | GGGTCTCTGTGCGAG | GCACAAAGGGCTCTC | 138 |
| | | rs13044266 | AGG | CGAAG | |
| | | | (SEQ ID NO: 615) | (SEQ ID NO: 616) | |
| NM_005560.6 | 20 | rs477859 | GGACCTTTACTGCAA | CGTGCCATCGATGGC | 139 |
| | | | GCTGGTA | ATTG | |
| | | | (SEQ ID NO: 617) | (SEQ ID NO: 618) | |
| NM_005560.6 | 20 | rs13038331 | GGGCTACGCGCAGAT | GACCCGCCCGCTCAC | 127 |
| | | | GGC | ACTC | |
| | | | (SEQ ID NO: 619) | (SEQ ID NO: 620) | |
| NM_001085455.3 | 21 | rs2832753 | CACCTCTGAGGTATT | AGATTCAGCAGAAAA | 137 |
| | | | TGTGCAG | TTAGACGTTCT | |
| | | | (SEQ ID NO: 621) | (SEQ ID NO: 622) | |
| NM_001085455.3 | 21 | rs2832752 | AATACAACCATCACC | CCATCCGAATGAATT | 138 |
| | | | GGCTTT | TGCATGCT | |
| | | | (SEQ ID NO: 623) | (SEQ ID NO: 624) | |
| NM_181606.3 | 21 | rs9982775 | CCACCATGACTCGTT | TCCACAGCCATAGTT | 140 |
| | | | ACTTCTG | CAGGG | |
| | | | (SEQ ID NO: 625) | (SEQ ID NO: 626) | |
| NM_181606.3 | 21 | rs9982675, | GGCTGTGGATGCAAT | TCAGTAGGTGCTGTA | 138 |
| | | rs9982755, | GGCT | GCCAA | |
| | | rs9982910 | (SEQ ID NO: 627) | (SEQ ID NO: 628) | |
| NM_181684.3 | 21 | rs13046903, | CAGCTGCTCCTCGGG | CTTGAAGCTCACGGG | 142 |
| | | rs7275281, | CTG | CACA | |
| | | rs7275298, | (SEQ ID NO: 629) | (SEQ ID NO: 630) | |
| | | rs7276859 | | | |
| NM_181684.3 | 21 | rs12481809, | TGTGAGCTGCAGGCC | AGCGGGTCTGGAGAT | 142 |
| | | rs12483730, | TGT | TCATG | |
| | | rs2838622 | (SEQ ID NO: 631) | (SEQ ID NO: 632) | |
| NM_181686.2 | 21 | rs7283084, | ACACGCCACCATGTG | CACGGCTGGCTTGAA | 142 |
| | | rs9984476 | CCA | GCT | |
| | | | (SEQ ID NO: 633) | (SEQ ID NO: 634) | |
| NM_181686.2 | 21 | rs2838621 | GCTGCTCCCAGTGCA | CCAGATCATTCTATT | 140 |
| | | | GATG | ATATTCTCGATCCAG | |
| | | | (SEQ ID NO: 635) | (SEQ ID NO: 636) | |
| NM_198688.3 | 21 | rs233303 | GGATTCCTCTTCATG | CAAGCCGGCTGGCAG | 141 |
| | | | CTGCCA | CTA | |
| | | | (SEQ ID NO: 637) | (SEQ ID NO: 638) | |
| NM_198688.3 | 21 | rs58757665, | GCTCCTGCACACCCT | GAGGAATCCTCAGAG | 139 |
| | | rs9980630 | CATG | CAGGTG | |
| | | | (SEQ ID NO: 639) | (SEQ ID NO: 640) | |
| NM_198688.3 | 21 | rs13051603 | TGCCAGCTGGCTTGC | ACTGCTGGCAGCATG | 133 |
| | | | TGT | AAGAA | |
| | | | (SEQ ID NO: 641) | (SEQ ID NO: 642) | |
| NM_198688.3 | 21 | rs201732843, | TGACTCTTGCTCCGA | CAGGTCACTGGGCAG | 141 |
| | | rs233309 | CTCCT | CAG | |
| | | | (SEQ ID NO: 643) | (SEQ ID NO: 644) | |
| NM_203405.2 | 21 | rs2236427 | GCTAGTAGCAACTGA | CCAGAGCAACATTGA | 137 |
| | | | TGTTTCTTAGT | ATAGCATTG | |
| | | | (SEQ ID NO: 645) | (SEQ ID NO: 646) | |
| NM_203405.2 | 21 | rs3804008 | AGCACCACCTTCTAG | AGTTTCCCGAGCAGT | 138 |
| | | | GGATAAATAAA | AGTTGG | |
| | | | (SEQ ID NO: 647) | (SEQ ID NO: 648) | |
| NM_203405.2 | 21 | rs3804007 | GGGAAACTCCAACTC | GTCTGTGACCCAGGT | 139 |
| | | | AGGATCT | ATGGT | |
| | | | (SEQ ID NO: 649) | (SEQ ID NO: 650) | |
| NM_203405.2 | 21 | rs78731676 | CTATTAAAACTCAGT | TGTATGAAGATGCAT | 135 |
| | | | CGCATTGGTTT | CAGAACTCTT | |
| | | | (SEQ ID NO: 651) | (SEQ ID NO: 652) | |

### Example 2

A high-throughput method for detecting RNA polymorphisms for individual identification of hair shaft samples included the following steps:

### 1. Extraction and Reverse Transcription of Hair Shaft RNA

(1) Pre-treatment: approximately 5 centimetre (cm)-long hair shaft samples were cut, and the 1 cm segment near the follicle end was removed. A total of about 20 mg hair shaft samples was collected and placed in a 1.5 mL nuclease-free centrifuge tube. The hair shafts were cut into approximately 3 mm fragments using scissors and sequentially washed with anhydrous ethanol, nuclease-free water, and anhydrous ethanol, after which they were air-dried.

(2) Grinding: the hair shafts obtained in step (1) were ground using a cryogenic grinder under the following conditions: 60 Hz, 60 s, repeated 2 times.

(3) Digestion: the ground hair shafts from step (2) were digested with 230 μL of digestion buffer (50 μL of 1% SDS, 50 μL of 1 M Tris-HCl, 50 μL of 0.5 M EDTA, 50 μL of 3% NaCl, 20 μL of 1 M DTT, and 10 μL of proteinase K (PK)). The constant-temperature metal bath was set to the following conditions: 12,000 rpm, 58° C. for 5-10 min.

(4) RNA Extraction: total RNA from the sample obtained in step (3) was extracted using RNAiso Plus reagent. The specific procedures were as follows:
1) 1 mL of RNAiso Plus reagent was added to the digested nuclease-free centrifuge tube, mixed manually, and allowed to stand for 5 min;
2) 200 μL of chloroform was added, thoroughly mixed, and allowed to stand for 10 min;
3) the mixture was centrifuged at 4° C. and 12,000 rpm for 15 min, a resulting supernatant was aspirated after centrifugation, and transferred to a new nuclease-free centrifuge tube;
4) an equal volume of isopropanol was added, mixed by inversion, and precipitated at −20° C. for 2 h;
5) the centrifuge tube was centrifuged at 4° C. and 12,000 rpm for 20 min, a resulting supernatant was discarded, and 1 mL of 75% ethanol was added; after several inversions, the centrifuge tube was centrifuged again at 4° C. and 12,000 rpm for 20 min, and a resulting supernatant was discarded; and
6) the lid of the nuclease-free centrifuge tube was opened, and the RNA was air-dried at room temperature and dissolved in 20 μL of nuclease-free water.

(5) RNA Reverse Transcription: a HiFiScript gDNA Removal RT MasterMix reverse transcription kit was used for the operation.

### 2. High-Throughput Detection System Design

In this example, 78 highly-expressed mRNA markers in hair shafts were screened from existing whole transcriptome sequencing data, containing 404 coding SNP (cSNP) sites. The site information was shown in Table 1.

The high-throughput multiplex detection system was designed by Shanghai Dynegene Technologies Co., Ltd., including a total of 326 amplicons. Among these, 61 amplicons contained 2 or more cSNP sites, referred to as microhaplotypes (MHs) in the present disclosure. The primer sequences were listed in Table 2.

### 3. High-Throughput Sequencing Library Construction

Library construction was completed using the QuarMultiple PCR Capture Kit according to the manufacturer's instructions.

### 4. Sequencing

Sequencing was conducted using the DNBSEQ™ platform with PE150 mode.

### 5. Result Analysis

Due to the micro-degradation nature of hair shaft specimen, the “reads” count threshold of amplicons was set to 30 during result analysis. Following data analysis procedures referenced in literature (Liu Z, Wang J, Li L, Yang H, Yu H, Fan J, Zhang M, Zhang Y, Liu J, Li Z, Zhang G. Identification of the body fluid donor in mixtures through target mRNA cSNP sequencing. Forensic Sci Int Genet. 2024 July; 71:103066. doi: 10.1016/j.fsigen 2024.103066. Epub 2024 May 31. PMID: 38833776), the sequencing results were analyzed using the transcript sequences of all genes (sequences corresponding to the NM numbers in Table 1) as reference sequences to obtain genotyping results for the cSNP/MH sites.

### Example 3

### 1. Polymorphism Detection of the Hair Shaft RNA Polymorphism High-Throughput Detection Kit in a Shanxi Population

40 hair shaft samples were collected from the Shanxi region. RNA extraction, reverse transcription, library construction, sequencing, and analysis were conducted according to the methods described in Example 2 to obtain population data for each site in this multiplex detection kit. After analysis, 97 amplicons in this kit did not meet the “reads” count threshold of >30 in any sample. The final dataset included 286 cSNPs located on 71 genes, contained in 229 amplicons, including 45 MHs. Among the sites that met the analysis criteria, 18 amplicons showed no polymorphism in the population, while the remaining amplicons exhibited 2 to 5 alleles. Allele frequencies and genotype frequencies were shown in Tables 3 to 4.

TABLE 3
Allele frequencies of cSNP/MH sites

| | Observed | Allele | | Observed | Allele |
|---|---|---|---|---|---|
| cSNP/MH markers | allele | frequency | cSNP/MH markers | allele | frequency |
| rs11464 | A | 1.00 | rs4643041 | A | 0.80 |
| rs1063128 | A | 0.96 | | C | 0.20 |
| | G | 0.04 | rs12233086 | A | 0.86 |
| rs3820553 | A | 0.95 | | G | 0.14 |
| | G | 0.05 | rs7558074 | A | 0.57 |
| rs6696123 | A | 0.96 | | C | 0.43 |
| | G | 0.04 | rs11896520 | C | 1.00 |
| rs2273584 | A | 0.06 | rs1376443 | C | 0.38 |
| | G | 0.94 | | T | 0.62 |
| rs11549949, rs11549950 | AG | 1.00 | rs9308819 | A | 0.03 |
| rs486903 | C | 0.64 | | G | 0.97 |
| | T | 0.36 | rs1129235, rs8121 | AA | 0.78 |
| rs12896 | C | 0.80 | | GC | 0.22 |
| | T | 0.20 | rs3173082 | C | 0.19 |
| rs1883 | A | 0.75 | | T | 0.81 |
| | G | 0.25 | rs7745062 | C | 0.21 |
| rs8020483 | C | 0.73 | | T | 0.79 |
| | T | 0.27 | rs7745259 | C | 0.06 |
| rs10289 | A | 0.26 | | T | 0.94 |
| | G | 0.74 | rs3777667 | A | 0.11 |
| rs4246 | A | 0.83 | | G | 0.89 |
| | C | 0.17 | rs17152433 | A | 0.24 |
| rs1047448 | A | 0.95 | | G | 0.76 |
| | G | 0.05 | rs3906796 | C | 1.00 |
| rs1047449 | A | 0.04 | rs3845 | C | 0.75 |
| | C | 0.96 | | T | 0.25 |
| rs3744786 | A | 0.45 | rs142478896, rs143258620, | AGGA | 1.00 |
| | | | rs145108464, rs148576335 | | |
| | G | 0.55 | rs1864253 | C | 0.68 |
| rs2071561 | A | 0.80 | | G | 0.32 |
| | C | 0.20 | rs150285041 | C | 1.00 |
| rs72830046 | A | 0.07 | rs6435927 | G | 1.00 |
| | G | 0.93 | rs1071862 | C | 0.50 |
| rs12948056 | A | 0.08 | | G | 0.50 |
| | G | 0.92 | rs76239019 | C | 0.50 |
| rs11078993, rs2857257 | CA | 0.17 | | T | 0.50 |
| | CG | 0.31 | rs10186663 | A | 0.86 |
| | TA | 0.10 | | G | 0.14 |
| | TG | 0.43 | rs1193 | C | 0.14 |
| rs2071563, rs2604954, | CAC | 0.50 | | T | 0.86 |
| rs2604955 | CGT | 0.20 | rs13297 | A | 0.85 |
| | TGT | 0.30 | | G | 0.15 |
| rs2604953 | A | 0.70 | rs1049457 | C | 0.13 |
| | C | 0.30 | | T | 0.87 |
| rs71373411 | G | 0.04 | rs11547281 | A | 0.13 |
| | T | 0.96 | | C | 0.88 |
| rs743686 | C | 0.56 | rs818571 | C | 0.49 |
| | T | 0.44 | | T | 0.51 |
| rs7216017 | C | 0.45 | rs74506939 | C | 1.00 |
| | T | 0.55 | rs112542604, rs141856614 | TC | 1.00 |
| rs12451652, rs2071601, | ACC | 0.28 | rs3815561 | C | 0.73 |
| rs2239711 | GCT | 0.22 | | T | 0.28 |
| | GGC | 0.50 | rs3829598, rs9890989, | ATC | 0.56 |
| rs79897879 | A | 0.96 | rs9897046 | ATT | 0.23 |
| | C | 0.04 | | GCC | 0.21 |
| rs2071588 | C | 0.20 | rs3813048, rs3813050 | CA | 0.53 |
| | G | 0.80 | | TA | 0.01 |
| rs3187034, rs7300801 | CG | 0.68 | | TG | 0.46 |
| | TA | 0.33 | rs3813047 | C | 0.79 |
| rs4761784, rs4761856 | CA | 0.03 | | T | 0.21 |
| | CG | 0.64 | rs375280428 | C | 0.53 |
| | TA | 0.34 | | T | 0.48 |
| rs3657 | A | 0.99 | rs147906218, rs76327324, | AGC | 0.08 |
| | G | 0.01 | rs80168553 | CAC | 0.41 |
| rs2857663 | C | 0.95 | | CGC | 0.51 |
| | T | 0.05 | rs72625995, rs77163010 | AC | 0.04 |
| rs3741715 | C | 0.49 | | AT | 0.53 |
| | T | 0.51 | | GC | 0.44 |
| rs2857667 | C | 0.99 | rs7212517, rs72625994 | AG | 0.21 |
| | T | 0.01 | | AT | 0.44 |
| rs2852464, rs2857669 | CC | 0.86 | | GG | 0.35 |
| | CG | 0.01 | rs7182648 | C | 0.73 |
| | GT | 0.13 | | T | 0.27 |
| rs2257286 | C | 0.96 | rs1280396 | A | 0.14 |
| | T | 0.04 | | G | 0.86 |
| rs2248473, rs2852468 | AT | 0.93 | rs1620402 | G | 0.12 |
| | GT | 0.07 | | T | 0.88 |
| rs2857670 | C | 0.68 | rs16977594 | A | 0.97 |
| | T | 0.32 | | G | 0.03 |
| rs2857671 | C | 0.36 | rs2131730, rs2131731 | AA | 0.93 |
| | T | 0.64 | | TG | 0.07 |
| rs7968466 | A | 0.35 | rs1666573 | C | 0.07 |
| | G | 0.65 | | T | 0.93 |
| rs1059837 | C | 0.53 | rs2934418 | A | 0.89 |
| | T | 0.48 | | C | 0.11 |
| rs15667 | A | 0.59 | rs3742966, rs937248 | CA | 0.03 |
| | G | 0.41 | | CG | 0.53 |
| rs2302613, rs2302614 | GA | 0.69 | | TG | 0.44 |
| | TA | 0.06 | rs3742963, rs3742964 | AT | 0.92 |
| | TG | 0.25 | | TC | 0.08 |
| rs1049500 | C | 0.37 | rs7166305 | A | 0.11 |
| | T | 0.63 | | C | 0.89 |
| rs201017624 | C | 1.00 | rs11856, rs8784 | AC | 0.12 |
| rs1876363 | C | 1.00 | | GC | 0.51 |
| rs1049205 | A | 0.70 | | GT | 0.37 |
| | G | 0.30 | rs12439292 | G | 0.08 |
| rs2075639 | C | 0.77 | | T | 0.92 |
| | T | 0.23 | rs12452295 | C | 0.75 |
| rs1049206 | A | 0.31 | | T | 0.25 |
| | G | 0.69 | rs8072775 | A | 0.44 |
| rs1049207, rs1049208 | CC | 0.01 | | G | 0.56 |
| | CT | 0.67 | rs2178428 | C | 0.03 |
| | GC | 0.32 | | T | 0.97 |
| rs2037912 | C | 0.37 | rs2007922 | A | 0.90 |
| | G | 0.63 | | G | 0.10 |
| rs1049212 | C | 0.68 | rs923935 | A | 0.98 |
| | T | 0.32 | | G | 0.03 |
| rs7214 | A | 0.65 | rs923936 | C | 0.97 |
| | C | 0.35 | | T | 0.03 |
| rs1128416 | A | 0.85 | rs936524 | C | 0.58 |
| | G | 0.15 | | T | 0.42 |
| rs11687800 | C | 0.12 | rs12983010 | C | 0.05 |
| | T | 0.88 | | T | 0.95 |
| rs7475 | A | 0.97 | rs73038948 | A | 1.00 |
| | G | 0.03 | rs4801861 | C | 0.60 |
| rs7908387 | C | 0.94 | | T | 0.40 |
| | T | 0.06 | rs1060186 | C | 0.59 |
| rs1681 | C | 0.25 | | T | 0.41 |
| | T | 0.75 | rs382367, rs3894847 | AG | 0.03 |
| rs2076190 | G | 0.58 | | CG | 0.97 |
| | T | 0.42 | rs3741748 | C | 1.00 |
| rs2231301 | A | 0.74 | rs77813527 | A | 1.00 |
| | G | 0.26 | rs1797683 | C | 0.61 |
| rs1950252 | A | 0.03 | | T | 0.39 |
| | G | 0.97 | rs11755593, rs2296744 | CG | 0.10 |
| rs10149339 | A | 0.99 | | TC | 0.90 |
| | G | 0.01 | rs10947436, rs2296743 | AC | 0.75 |
| rs12937519 | C | 0.96 | | GC | 0.25 |
| | T | 0.04 | rs9982775 | C | 0.41 |
| rs16966703 | C | 0.03 | | T | 0.59 |
| | T | 0.98 | rs9982675, rs9982755, | CGA | 0.40 |
| rs2076304 | A | 0.15 | rs9982910 | TTC | 0.60 |
| | G | 0.85 | rs13046903, rs7275281, | ATCG | 0.61 |
| rs2064217 | C | 0.83 | rs7275298, rs7276859 | ATTC | 0.05 |
| | T | 0.17 | | GCTC | 0.34 |
| rs2076299 | A | 0.82 | rs12481809, rs12483730, | CGC | 0.64 |
| | G | 0.18 | rs2838622 | CGT | 0.31 |
| rs6929069 | A | 0.18 | | TAT | 0.05 |
| | G | 0.83 | rs7283084, rs9984476 | AA | 0.03 |
| rs2076300 | C | 0.93 | | GA | 0.46 |
| | T | 0.07 | | GG | 0.51 |
| rs2744380 | C | 0.76 | rs2838621 | A | 0.56 |
| | G | 0.24 | | G | 0.44 |
| rs12250 | C | 0.83 | rs4965046 | C | 0.91 |
| | T | 0.17 | | G | 0.09 |
| rs3745951 | C | 0.13 | rs112100339, rs12101554 | CA | 0.25 |
| | T | 0.88 | | CG | 0.75 |
| rs34989303 | A | 0.12 | rs2130882 | C | 0.49 |
| | G | 0.88 | | T | 0.51 |
| rs3745952 | C | 0.88 | rs3803385 | A | 0.26 |
| | T | 0.13 | | G | 0.74 |
| rs931750 | C | 0.13 | rs3169159, rs3169160 | CA | 0.83 |
| | T | 0.87 | | CG | 0.11 |
| rs931749 | C | 0.89 | | TG | 0.06 |
| | T | 0.11 | rs9652498 | A | 0.11 |
| rs73041302 | A | 0.88 | | C | 0.89 |
| | G | 0.13 | rs1043372 | T | 1.00 |
| rs61160177 | C | 0.59 | rs1043374 | C | 1.00 |
| | G | 0.41 | rs201732843, rs233309 | CG | 0.88 |
| rs77862444 | C | 0.97 | | TA | 0.12 |
| | T | 0.03 | rs13051603 | C | 0.96 |
| rs8106900 | G | 0.88 | | T | 0.04 |
| | T | 0.13 | rs233303 | C | 0.92 |
| rs4386247 | C | 0.17 | | T | 0.08 |
| | T | 0.83 | rs3804008 | G | 0.01 |
| rs118185142, rs12609247 | CG | 0.41 | | T | 0.99 |
| | TA | 0.13 | rs3804007 | A | 0.01 |
| | TG | 0.46 | | C | 0.99 |
| rs1142825 | A | 0.60 | rs2236427 | G | 0.41 |
| | G | 0.40 | | T | 0.59 |
| rs4242797 | A | 1.00 | rs3818831 | C | 0.42 |
| rs4589188, rs4589189 | CA | 0.14 | | T | 0.58 |
| | GG | 0.86 | rs2832753 | A | 0.76 |
| rs1131482 | G | 0.37 | | G | 0.24 |
| | T | 0.63 | rs2832752 | C | 0.77 |
| rs4947 | C | 0.35 | | G | 0.23 |
| | T | 0.65 | rs3781679 | C | 0.38 |
| rs1059623 | C | 0.16 | | T | 0.63 |
| | T | 0.84 | rs11205896 | A | 0.20 |
| rs10792769 | A | 0.89 | | C | 0.80 |
| | G | 0.11 | rs1770791, rs8375 | CC | 0.17 |
| rs2663, rs2664 | CA | 0.48 | | CT | 0.04 |
| | CG | 0.09 | | TC | 0.13 |
| | TG | 0.43 | | TT | 0.65 |
| rs2665 | A | 0.16 | rs6503578, rs9635728 | CC | 0.50 |
| | T | 0.84 | | GC | 0.50 |
| rs4755571 | A | 0.70 | rs6503577 | A | 0.56 |
| | G | 0.30 | | T | 0.44 |
| rs2074478 | A | 0.79 | rs10788862 | A | 0.14 |
| | G | 0.21 | | T | 0.86 |
| rs1265099, rs3132558 | CC | 0.43 | rs9863910 | C | 0.06 |
| | CT | 0.33 | | T | 0.94 |
| | GC | 0.09 | rs576 | C | 0.79 |
| | GT | 0.16 | | T | 0.21 |
| rs626974 | C | 0.15 | rs704246 | C | 0.82 |
| | T | 0.85 | | T | 0.18 |
| rs564196 | A | 0.14 | rs4946758 | A | 0.43 |
| | G | 0.86 | | G | 0.58 |
| rs611251 | C | 0.09 | rs1340623, rs4945755, | ACG | 0.15 |
| | T | 0.91 | rs4946759 | AGG | 0.22 |
| rs557806, rs610308 | GA | 1.00 | | GGA | 0.61 |
| rs11541192, rs556052 | AC | 0.04 | | GGG | 0.02 |
| | GC | 0.09 | rs11152999 | A | 0.55 |
| | GG | 0.87 | | G | 0.45 |
| rs500079 | A | 0.87 | rs1159148 | A | 0.93 |
| | G | 0.13 | | C | 0.07 |
| rs524, rs527 | CG | 0.87 | rs3747787 | C | 0.05 |
| | TC | 0.13 | | T | 0.95 |
| rs558614 | C | 0.48 | rs2297970 | A | 0.04 |
| | T | 0.52 | | G | 0.96 |
| rs2304704 | C | 0.85 | rs3747790 | C | 0.19 |
| | T | 0.15 | | T | 0.81 |
| rs17337252 | C | 0.86 | rs11537704 | C | 0.87 |
| | T | 0.14 | | T | 0.13 |
| rs2305427 | C | 0.80 | rs1135384, rs6922741, | GTA | 0.06 |
| | T | 0.20 | rs9486407 | TCG | 0.94 |
| rs1129660 | C | 0.81 | rs13319 | A | 0.08 |
| | T | 0.19 | | G | 0.92 |
| rs77685055 | C | 0.91 | rs1057433 | C | 0.92 |
| | T | 0.09 | | T | 0.08 |
| rs3740304 | C | 0.08 | rs8013 | A | 0.90 |
| | T | 0.92 | | C | 0.10 |
| rs2640738 | A | 0.73 | rs2239710 | C | 0.63 |
| | G | 0.27 | | T | 0.37 |
| rs2640785 | A | 0.58 | rs2071599 | A | 0.86 |
| | T | 0.42 | | G | 0.14 |
| rs2846412 | A | 0.88 | rs1051602 | A | 0.98 |
| | G | 0.12 | | G | 0.03 |
| rs10749920 | C | 0.23 | rs6436712 | A | 0.54 |
| | T | 0.77 | | G | 0.46 |
| rs2640779 | A | 0.61 | rs112558913, rs113140754 | GC | 0.01 |
| | G | 0.39 | | GT | 0.97 |
| rs3741060 | C | 0.20 | | TT | 0.01 |
| | T | 0.80 | rs113823535 | A | 0.05 |
| — | — | — | | G | 0.95 |

TABLE 4
Genotype frequencies of RNA cSNP/MH markers

| | Hapelotype | | | Hapelotype | |
|---|---|---|---|---|---|
| cSNP/MH markers | frequency | Frequency | cSNP/MH markers | frequency | Frequency |
| rs11464 | A/A | 1.00 | rs2640779 | A/A | 0.38 |
| rs1063128 | A/A | 0.92 | | G/G | 0.15 |
| | G/G | 0.00 | | A/G | 0.47 |
| | A/G | 0.08 | rs3741060 | C/C | 0.04 |
| rs3820553 | A/A | 0.91 | | T/T | 0.64 |
| | G/G | 0.00 | | C/T | 0.32 |
| | A/G | 0.09 | rs4643041 | A/A | 0.64 |
| rs6696123 | A/A | 0.93 | | C/C | 0.04 |
| | G/G | 0.00 | | A/C | 0.32 |
| | A/G | 0.07 | rs12233086 | A/A | 0.73 |
| rs2273584 | A/A | 0.00 | | G/G | 0.02 |
| | G/G | 0.89 | | A/G | 0.24 |
| | A/G | 0.10 | rs7558074 | A/A | 0.32 |
| rs11549949, rs11549950 | AG/AG | 1.00 | | C/C | 0.19 |
| rs486903 | C/C | 0.41 | | A/C | 0.49 |
| | T/T | 0.13 | rs11896520 | C/C | 1.00 |
| | C/T | 0.46 | rs1376443 | C/C | 0.15 |
| rs12896 | C/C | 0.64 | | T/T | 0.38 |
| | T/T | 0.04 | | C/T | 0.47 |
| | C/T | 0.32 | rs9308819 | A/A | 0.00 |
| rs1883 | A/A | 0.56 | | G/G | 0.94 |
| | G/G | 0.06 | | A/G | 0.06 |
| | A/G | 0.38 | rs1129235, rs8121 | AA/AA | 0.67 |
| rs8020483 | C/C | 0.53 | | GC/GC | 0.11 |
| | T/T | 0.07 | | AA/GC | 0.22 |
| | C/T | 0.39 | rs3173082 | C/C | 0.04 |
| rs10289 | A/A | 0.07 | | T/T | 0.66 |
| | G/G | 0.55 | | C/T | 0.30 |
| | A/G | 0.38 | rs7745062 | C/C | 0.04 |
| rs4246 | A/A | 0.69 | | T/T | 0.63 |
| | C/C | 0.03 | | C/T | 0.33 |
| | A/C | 0.28 | rs7745259 | C/C | 0.00 |
| rs1047448 | A/A | 0.91 | | T/T | 0.89 |
| | G/G | 0.00 | | C/T | 0.10 |
| | A/G | 0.09 | rs3777667 | A/A | 0.01 |
| rs1047449 | A/A | 0.00 | | G/G | 0.79 |
| | C/C | 0.93 | | A/G | 0.20 |
| | A/C | 0.07 | rs17152433 | A/A | 0.06 |
| rs3744786 | A/A | 0.20 | | G/G | 0.57 |
| | G/G | 0.30 | | A/G | 0.37 |
| | A/G | 0.50 | rs3906796 | C/C | 1.00 |
| rs2071561 | A/A | 0.64 | rs3845 | C/C | 0.56 |
| | C/C | 0.04 | | T/T | 0.06 |
| | A/C | 0.32 | | C/T | 0.38 |
| rs72830046 | A/A | 0.00 | rs142478896, rs143258620, | AGGA/AGGA | 1.00 |
| | | | rs145108464, rs148576335 | | |
| | G/G | 0.87 | rs1864253 | C/C | 0.47 |
| | A/G | 0.13 | | G/G | 0.10 |
| rs12948056 | A/A | 0.01 | | C/G | 0.43 |
| | G/G | 0.84 | rs150285041 | C/C | 1.00 |
| | A/G | 0.15 | rs6435927 | G/G | 1.00 |
| rs11078993, rs2857257 | TG/TG | 0.19 | rs1071862 | C/C | 0.25 |
| | TG/CG | 0.33 | | G/G | 0.25 |
| | CG/TA | 0.19 | | C/G | 0.50 |
| | CG/CG | 0.05 | rs76239019 | C/C | 0.25 |
| | TG/CA | 0.14 | | T/T | 0.25 |
| | CA/CA | 0.10 | | C/T | 0.50 |
| rs2071563, rs2604954, | TGT/CAC | 0.22 | rs10186663 | A/A | 0.74 |
| rs2604955 | CGT/TGT | 0.19 | | G/G | 0.02 |
| | TGT/TGT | 0.09 | | A/G | 0.24 |
| | CAC/CAC | 0.31 | rs1193 | C/C | 0.02 |
| | CGT/CAC | 0.16 | | T/T | 0.74 |
| | CGT/CGT | 0.03 | | C/T | 0.24 |
| rs2604953 | A/A | 0.50 | rs13297 | A/A | 0.72 |
| | C/C | 0.09 | | G/G | 0.02 |
| | A/C | 0.42 | | A/G | 0.26 |
| rs71373411 | G/G | 0.00 | rs1049457 | C/C | 0.02 |
| | T/T | 0.93 | | T/T | 0.75 |
| | G/T | 0.07 | | C/T | 0.23 |
| rs743686 | C/C | 0.32 | rs11547281 | A/A | 0.02 |
| | T/T | 0.19 | | C/C | 0.77 |
| | C/T | 0.49 | | A/C | 0.22 |
| rs7216017 | C/C | 0.20 | rs818571 | C/C | 0.24 |
| | T/T | 0.30 | | T/T | 0.26 |
| | C/T | 0.50 | | C/T | 0.50 |
| rs12451652, rs2071601, | GCT/GGC | 0.33 | rs74506939 | C/C | 1.00 |
| rs2239711 | ACC/GGC | 0.22 | rs112542604, rs141856614 | TC/TC | 1.00 |
| | GGC/GGC | 0.22 | rs3815561 | C/C | 0.53 |
| | ACC/ACC | 0.11 | | T/T | 0.08 |
| | GCT/ACC | 0.11 | | C/T | 0.40 |
| rs79897879 | A/A | 0.93 | rs3829598, rs9890989, | ATC/ATC | 0.28 |
| | C/C | 0.00 | rs9897046 | GCC/ATT | 0.13 |
| | A/C | 0.07 | | ATT/ATC | 0.33 |
| rs2071588 | C/C | 0.04 | | GCC/GCC | 0.03 |
| | G/G | 0.64 | | ATC/GCC | 0.25 |
| | C/G | 0.32 | rs3813048, rs3813050 | CA/TG | 0.83 |
| rs3187034, rs7300801 | CG/TA | 0.65 | | TG/TG | 0.05 |
| | CG/CG | 0.35 | | CA/TA | 0.03 |
| rs4761784, rs4761856 | CG/TA | 0.51 | | CA/CA | 0.10 |
| | CG/CG | 0.35 | rs3813047 | C/C | 0.62 |
| | TA/TA | 0.05 | | T/T | 0.05 |
| | TA/TG | 0.03 | | C/T | 0.33 |
| | CG/CA | 0.05 | rs375280428 | C/C | 0.28 |
| rs3657 | A/A | 0.97 | | T/T | 0.23 |
| | G/G | 0.00 | | C/T | 0.50 |
| | A/G | 0.03 | rs147906218, rs76327324, | CGC/CAC | 0.75 |
| rs2857663 | C/C | 0.90 | rs80168553 | CGC/CGC | 0.10 |
| | T/T | 0.00 | | CGC/AGC | 0.08 |
| | C/T | 0.10 | | AGC/CAC | 0.08 |
| rs3741715 | C/C | 0.24 | rs72625995, rs77163010 | AT/AC | 0.08 |
| | T/T | 0.26 | | AT/GC | 0.88 |
| | C/T | 0.50 | | AT/AT | 0.05 |
| rs2857667 | C/C | 0.98 | rs7212517, rs72625994 | GG/GG | 0.05 |
| | T/T | 0.00 | | AG/AT | 0.33 |
| | C/T | 0.02 | | GG/AT | 0.50 |
| rs2852464, rs2857669 | CC/CC | 0.78 | | GG/AG | 0.10 |
| | CC/GT | 0.18 | | AT/AT | 0.03 |
| | GT/GT | 0.03 | rs7182648 | C/C | 0.53 |
| | CG/GT | 0.03 | | T/T | 0.08 |
| rs2257286 | C/C | 0.93 | | C/T | 0.40 |
| | T/T | 0.00 | rs1280396 | A/A | 0.02 |
| | C/T | 0.07 | | G/G | 0.73 |
| rs2248473, rs2852468 | AT/AT | 0.93 | | A/G | 0.25 |
| | AT/GT | 0.08 | rs1620402 | G/G | 0.01 |
| rs2857670 | C/C | 0.46 | | T/T | 0.78 |
| | T/T | 0.11 | | G/T | 0.20 |
| | C/T | 0.44 | rs16977594 | A/A | 0.95 |
| rs2857671 | C/C | 0.13 | | G/G | 0.00 |
| | T/T | 0.41 | | A/G | 0.05 |
| | C/T | 0.46 | rs2131730, rs2131731 | AA/AA | 0.87 |
| rs7968466 | A/A | 0.12 | | AA/TG | 0.13 |
| | G/G | 0.43 | rs1666573 | C/C | 0.01 |
| | A/G | 0.45 | | T/T | 0.86 |
| rs1059837 | C/C | 0.28 | | C/T | 0.13 |
| | T/T | 0.23 | rs2934418 | A/A | 0.79 |
| | C/T | 0.50 | | C/C | 0.01 |
| rs15667 | A/A | 0.35 | | A/C | 0.20 |
| | G/G | 0.17 | rs3742966, rs937248 | CG/TG | 0.18 |
| | A/G | 0.48 | | CG/CG | 0.41 |
| rs2302613, rs2302614 | GA/TG | 0.13 | | CG/CA | 0.06 |
| | GA/GA | 0.63 | | TG/TG | 0.35 |
| | TG/TG | 0.13 | rs3742963, rs3742964 | AT/AT | 0.84 |
| | TG/TA | 0.13 | | AT/TC | 0.16 |
| rs1049500 | C/C | 0.14 | rs7166305 | A/A | 0.01 |
| | T/T | 0.39 | | C/C | 0.79 |
| | C/T | 0.47 | | A/C | 0.20 |
| rs201017624 | C/C | 1.00 | rs11856, rs8784 | GT/GC | 0.41 |
| rs1876363 | C/C | 1.00 | | GT/GT | 0.10 |
| rs1049205 | A/A | 0.50 | | GC/GC | 0.28 |
| | G/G | 0.09 | | GT/AC | 0.13 |
| | A/G | 0.42 | | AC/AC | 0.03 |
| rs2075639 | C/C | 0.60 | | AC/GC | 0.05 |
| | T/T | 0.05 | rs12439292 | G/G | 0.01 |
| | C/T | 0.35 | | T/T | 0.84 |
| rs1049206 | A/A | 0.09 | | G/T | 0.15 |
| | G/G | 0.48 | rs12452295 | C/C | 0.56 |
| | A/G | 0.43 | | T/T | 0.06 |
| rs1049207, rs1049208 | CT/CT | 0.53 | | C/T | 0.38 |
| | CT/GC | 0.28 | rs8072775 | A/A | 0.19 |
| | GC/CC | 0.03 | | G/G | 0.32 |
| | GC/GC | 0.17 | | A/G | 0.49 |
| rs2037912 | C/C | 0.13 | rs2178428 | C/C | 0.00 |
| | G/G | 0.40 | | T/T | 0.93 |
| | C/G | 0.46 | | C/T | 0.06 |
| rs1049212 | C/C | 0.46 | rs2007922 | A/A | 0.81 |
| | T/T | 0.10 | | G/G | 0.01 |
| | C/T | 0.44 | | A/G | 0.18 |
| rs7214 | A/A | 0.42 | rs923935 | A/A | 0.95 |
| | C/C | 0.12 | | G/G | 0.00 |
| | A/C | 0.46 | | A/G | 0.05 |
| rs1128416 | A/A | 0.73 | rs923936 | C/C | 0.95 |
| | G/G | 0.02 | | T/T | 0.00 |
| | A/G | 0.25 | | C/T | 0.05 |
| rs11687800 | C/C | 0.01 | rs936524 | C/C | 0.34 |
| | T/T | 0.78 | | T/T | 0.17 |
| | C/T | 0.21 | | C/T | 0.49 |
| rs7475 | A/A | 0.94 | rs12983010 | C/C | 0.00 |
| | G/G | 0.00 | | T/T | 0.89 |
| | A/G | 0.06 | | C/T | 0.10 |
| rs7908387 | C/C | 0.88 | rs73038948 | A/A | 1.00 |
| | T/T | 0.00 | rs4801861 | C/C | 0.36 |
| | C/T | 0.12 | | T/T | 0.16 |
| rs1681 | C/C | 0.06 | | C/T | 0.48 |
| | T/T | 0.56 | rs1060186 | C/C | 0.35 |
| | C/T | 0.38 | | T/T | 0.17 |
| rs2076190 | G/G | 0.34 | | C/T | 0.48 |
| | T/T | 0.18 | rs382367, rs3894847 | CG/CG | 0.93 |
| | G/T | 0.49 | | AG/CG | 0.07 |
| rs2231301 | A/A | 0.55 | rs3741748 | C/C | 1.00 |
| | G/G | 0.07 | rs77813527 | A/A | 1.00 |
| | A/G | 0.38 | rs1797683 | C/C | 0.37 |
| rs1950252 | A/A | 0.00 | | T/T | 0.16 |
| | G/G | 0.94 | | C/T | 0.48 |
| | A/G | 0.06 | rs11755593, rs2296744 | TC/TC | 0.80 |
| rs10149339 | A/A | 0.98 | | TC/CG | 0.20 |
| | G/G | 0.00 | rs10947436, rs2296743 | AC/AC | 0.63 |
| | A/G | 0.02 | | AC/GC | 0.25 |
| rs12937519 | C/C | 0.93 | | GC/GC | 0.13 |
| | T/T | 0.00 | rs9982775 | C/C | 0.17 |
| | C/T | 0.07 | | T/T | 0.35 |
| rs16966703 | C/C | 0.00 | | C/T | 0.48 |
| | T/T | 0.95 | rs9982675, rs9982755, | CGA/CGA | 0.13 |
| | C/T | 0.05 | rs9982910 | CGA/TTC | 0.54 |
| rs2076304 | A/A | 0.02 | | TTC/TTC | 0.33 |
| | G/G | 0.72 | rs13046903, rs7275281, | ATCG/ATCG | 0.48 |
| | A/G | 0.25 | rs7275298, rs7276859 | ATCG/GCTC | 0.28 |
| rs2064217 | C/C | 0.69 | | ATTC/GCTC | 0.10 |
| | T/T | 0.03 | | GCTC/GCTC | 0.15 |
| | C/T | 0.28 | rs12481809, rs12483730, | CGC/CGC | 0.45 |
| rs2076299 | A/A | 0.67 | rs2838622 | CGC/CGT | 0.33 |
| | G/G | 0.03 | | TAT/CGT | 0.03 |
| | A/G | 0.30 | | CGT/CGT | 0.13 |
| rs6929069 | A/A | 0.03 | | CGC/TAT | 0.08 |
| | G/G | 0.68 | rs7283084, rs9984476 | GG/GA | 0.87 |
| | A/G | 0.29 | | AA/GG | 0.05 |
| rs2076300 | C/C | 0.86 | | GG/GG | 0.05 |
| | T/T | 0.01 | | GA/GA | 0.03 |
| | C/T | 0.13 | rs2838621 | A/A | 0.32 |
| rs2744380 | C/C | 0.58 | | G/G | 0.19 |
| | G/G | 0.06 | | A/G | 0.49 |
| | C/G | 0.36 | rs4965046 | C/C | 0.82 |
| rs12250 | C/C | 0.69 | | G/G | 0.01 |
| | T/T | 0.03 | | C/G | 0.17 |
| | C/T | 0.28 | rs112100339, rs12101554 | CG/CG | 0.75 |
| rs3745951 | C/C | 0.02 | | CA/CA | 0.25 |
| | T/T | 0.77 | rs2130882 | C/C | 0.24 |
| | C/T | 0.22 | | T/T | 0.26 |
| rs34989303 | A/A | 0.01 | | C/T | 0.50 |
| | G/G | 0.77 | rs3803385 | A/A | 0.07 |
| | A/G | 0.21 | | G/G | 0.54 |
| rs3745952 | C/C | 0.77 | | A/G | 0.39 |
| | T/T | 0.02 | rs3169159, rs3169160 | CA/TG | 0.11 |
| | C/T | 0.22 | | CA/CG | 0.22 |
| rs931750 | C/C | 0.02 | | CA/CA | 0.67 |
| | T/T | 0.76 | rs9652498 | A/A | 0.01 |
| | C/T | 0.22 | | C/C | 0.79 |
| rs931749 | C/C | 0.79 | | A/C | 0.20 |
| | T/T | 0.01 | rs1043372 | T/T | 1.00 |
| | C/T | 0.20 | rs 1043374 | C/C | 1.00 |
| rs73041302 | A/A | 0.77 | rs201732843, rs233309 | CG/CG | 0.77 |
| | G/G | 0.02 | | CG/TA | 0.23 |
| | A/G | 0.22 | rs13051603 | C/C | 0.92 |
| rs61160177 | C/C | 0.35 | | T/T | 0.00 |
| | G/G | 0.17 | | C/T | 0.08 |
| | C/G | 0.48 | rs233303 | C/C | 0.84 |
| rs77862444 | C/C | 0.93 | | T/T | 0.01 |
| | T/T | 0.00 | | C/T | 0.15 |
| | C/T | 0.07 | rs3804008 | G/G | 0.00 |
| rs8106900 | G/G | 0.77 | | T/T | 0.97 |
| | T/T | 0.02 | | G/T | 0.03 |
| | G/T | 0.22 | rs3804007 | A/A | 0.00 |
| rs4386247 | C/C | 0.03 | | C/C | 0.98 |
| | T/T | 0.68 | | A/C | 0.02 |
| | C/T | 0.29 | rs2236427 | G/G | 0.17 |
| rs118185142, rs12609247 | CG/CG | 0.18 | | T/T | 0.35 |
| | TG/TA | 0.08 | | G/T | 0.48 |
| | TG/CG | 0.26 | rs3818831 | C/C | 0.17 |
| | TG/TG | 0.29 | | T/T | 0.34 |
| | TA/CG | 0.18 | | C/T | 0.49 |
| rs1142825 | A/A | 0.36 | rs2832753 | A/A | 0.57 |
| | G/G | 0.16 | | G/G | 0.06 |
| | A/G | 0.48 | | A/G | 0.37 |
| rs4242797 | A/A | 1.00 | rs2832752 | C/C | 0.60 |
| rs4589188, rs4589189 | GG/GG | 0.73 | | G/G | 0.05 |
| | GG/CA | 0.27 | | C/G | 0.35 |
| rs1131482 | G/G | 0.14 | rs3781679 | C/C | 0.14 |
| | T/T | 0.39 | | T/T | 0.39 |
| | G/T | 0.47 | | C/T | 0.47 |
| rs4947 | C/C | 0.12 | rs11205896 | A/A | 0.04 |
| | T/T | 0.42 | | C/C | 0.64 |
| | C/T | 0.46 | | A/C | 0.32 |
| rs1059623 | C/C | 0.03 | rs1770791, rs8375 | CT/TT | 0.08 |
| | T/T | 0.70 | | TT/TC | 0.19 |
| | C/T | 0.27 | | CC/TT | 0.19 |
| rs10792769 | A/A | 0.78 | | TT/TT | 0.42 |
| | G/G | 0.01 | | CC/CC | 0.08 |
| | A/G | 0.20 | | TC/TC | 0.04 |
| rs2663, rs2664 | TG/TG | 0.22 | rs6503578, rs9635728 | CC/GC | 1.00 |
| | CA/TG | 0.43 | rs6503577 | A/A | 0.32 |
| | CA/CA | 0.17 | | T/T | 0.19 |
| | CA/CG | 0.17 | | A/T | 0.49 |
| rs2665 | A/A | 0.02 | rs10788862 | A/A | 0.02 |
| | T/T | 0.71 | | T/T | 0.74 |
| | A/T | 0.27 | | A/T | 0.24 |
| rs4755571 | A/A | 0.49 | rs9863910 | C/C | 0.00 |
| | G/G | 0.09 | | T/T | 0.89 |
| | A/G | 0.42 | | C/T | 0.10 |
| rs2074478 | A/A | 0.62 | rs576 | C/C | 0.62 |
| | G/G | 0.05 | | T/T | 0.05 |
| | A/G | 0.33 | | C/T | 0.34 |
| rs1265099, rs3132558 | CC/CC | 0.23 | rs704246 | C/C | 0.67 |
| | CC/CT | 0.23 | | T/T | 0.03 |
| | CT/CT | 0.15 | | C/T | 0.30 |
| | CC/GC | 0.13 | rs4946758 | A/A | 0.18 |
| | GC/CT | 0.03 | | G/G | 0.33 |
| | GT/GC | 0.03 | | A/G | 0.49 |
| | GT/CT | 0.10 | rs1340623, rs4945755, | GGA/GGA | 0.39 |
| | GT/GT | 0.08 | rs4946759 | ACG/ACG | 0.09 |
| | CC/GT | 0.05 | | GGA/AGG | 0.35 |
| rs626974 | C/C | 0.02 | | ACG/GGA | 0.13 |
| | T/T | 0.73 | | AGG/AGG | 0.04 |
| | C/T | 0.25 | rs11152999 | A/A | 0.31 |
| rs564196 | A/A | 0.02 | | G/G | 0.20 |
| | G/G | 0.75 | | A/G | 0.49 |
| | A/G | 0.24 | rs1159148 | A/A | 0.87 |
| rs611251 | C/C | 0.01 | | C/C | 0.00 |
| | T/T | 0.83 | | A/C | 0.13 |
| | C/T | 0.16 | rs3747787 | C/C | 0.00 |
| rs557806, rs610308 | GA/GA | 1.00 | | T/T | 0.91 |
| rs11541192, rs556052 | GG/GG | 0.74 | | C/T | 0.09 |
| | GG/GC | 0.18 | rs2297970 | A/A | 0.00 |
| | GG/AC | 0.08 | | G/G | 0.93 |
| rs500079 | A/A | 0.76 | | A/G | 0.07 |
| | G/G | 0.02 | rs3747790 | C/C | 0.04 |
| | A/G | 0.22 | | T/T | 0.66 |
| rs524, rs527 | CG/CG | 0.74 | | C/T | 0.30 |
| | CG/TC | 0.26 | rs11537704 | C/C | 0.76 |
| rs558614 | C/C | 0.23 | | T/T | 0.02 |
| | T/T | 0.27 | | C/T | 0.23 |
| | C/T | 0.50 | rs1135384, rs6922741, | TCG/TCG | 0.87 |
| rs2304704 | C/C | 0.72 | rs9486407 | GTA/TCG | 0.13 |
| | T/T | 0.02 | rs13319 | A/A | 0.01 |
| | C/T | 0.26 | | G/G | 0.85 |
| rs17337252 | C/C | 0.74 | | A/G | 0.14 |
| | T/T | 0.02 | rs 1057433 | C/C | 0.85 |
| | C/T | 0.24 | | T/T | 0.01 |
| rs2305427 | C/C | 0.64 | | C/T | 0.15 |
| | T/T | 0.04 | rs8013 | A/A | 0.80 |
| | C/T | 0.32 | | C/C | 0.01 |
| rs1129660 | C/C | 0.66 | | A/C | 0.18 |
| | T/T | 0.04 | rs2239710 | C/C | 0.40 |
| | C/T | 0.30 | | T/T | 0.14 |
| rs77685055 | C/C | 0.82 | | C/T | 0.47 |
| | T/T | 0.01 | rs2071599 | A/A | 0.74 |
| | C/T | 0.17 | | G/G | 0.02 |
| rs3740304 | C/C | 0.01 | | A/G | 0.24 |
| | T/T | 0.84 | rs1051602 | A/A | 0.95 |
| | C/T | 0.15 | | G/G | 0.00 |
| rs2640738 | A/A | 0.53 | | A/G | 0.05 |
| | G/G | 0.07 | rs6436712 | A/A | 0.29 |
| | A/G | 0.39 | | G/G | 0.21 |
| rs2640785 | A/A | 0.34 | | A/G | 0.50 |
| | T/T | 0.17 | rs112558913, rs113140754 | GT/GT | 0.94 |
| | A/T | 0.49 | | GC/GT | 0.03 |
| rs2846412 | A/A | 0.78 | | GT/TT | 0.03 |
| | G/G | 0.01 | rs113823535 | A/A | 0.00 |
| | A/G | 0.20 | | G/G | 0.90 |
| rs10749920 | C/C | 0.05 | | A/G | 0.10 |
| | T/T | 0.59 | — | — | — |
| | C/T | 0.36 | — | — | — |

Due to significant variations among different hair shaft samples, among the 40 samples tested, the maximum number of amplicons detected was 218, and the minimum was 84, with an average of 167±27. The random match probability (RMP) of the system ranged from 4.71×10⁻³⁰ to 2.67×10⁻¹¹⁰, meeting the requirements for individual identification.

2. Consistency Testing of Genotyping Results from Hair Shafts of Different Body Parts

A total of 12 samples were collected, including hair shafts from the scalp, armpit, leg, and pubic region from 3 individuals. The genotyping results of armpit, leg, and pubic hair shafts were compared with those of scalp hair shafts using the system established in Example 2. As shown in Table 5, scalp and pubic hair shafts yielded more marker genotyping results than armpit and leg hair shafts, but the genotyping results of the co-detected markers were consistent.

TABLE 5
Detection results of hair shafts from different body parts

| | | | | | Co-detected |
|---|---|---|---|---|---|
| | | Pubic | | | and |
| | Scalp | region | Armpit | Leg | identically- |
| | hair | hair | hair | hair | genotyped |
| Samples | shaft | shaft | shaft | shaft | cSNPs/MHs |
| Sample 1 | 175 | 168 | 143 | 146 | 132 |
| Sample 2 | 189 | 183 | 155 | 172 | 142 |
| Sample 3 | 203 | 216 | 179 | 181 | 161 |
| Mean ± | 189 ± 14 | 189 ± 24.56 | 159 ± 18.33 | 166 ± 18.18 | 145 ± 14.73 |
| standard | | | | | |
| deviation | | | | | |

3. Detection of Typing Results from Hair Shafts with Different Colors

Black and white hair shafts from 2 individuals were collected and detected using the system established in Example 2. As shown in Table 6, black hair shafts yielded more than 141 amplicons, while white hair shafts yielded more than 161 amplicons. The number of co-detected amplicons ranged from 135 to 153, and all genotyping results for these co-detected amplicons were consistent.

TABLE 6
Detection results of hair shafts of different colors

| | Black hair | White hair | Co-detected | Identically-genotyped |
|---|---|---|---|---|
| Samples | shaft | shaft | cSNPs/MHs | cSNPs/MHs |
| Sample 1 | 160 | 199 | 153 | 153 |
| Sample 2 | 141 | 161 | 135 | 135 |

### 4. Species Specificity Testing

Hair shaft samples from four common animals, including rabbit, rat, cat, and dog, were collected and detected using the system established in Example 2. As shown in Table 7, the quantity of extracted RNA was 2.34±0.38 μg, indicating that the RNA extraction method for human hair shafts was also applicable to animal hair shafts. Some amplicons were detected in animal hair shafts, with dog hair shafts yielding the highest number (82 amplicons). However, the “reads” count of these amplicons was significantly lower than that in human hair shafts, and the cSNP/MH genotyping results differed from the reference sequences.

TABLE 7
Detection results of hair shafts from species

| Species | cSNP |
|---|---|
| Rabbit | 49 |
| Rat | 44 |
| Cat | 51 |
| Dog | 82 |

### 5. Detection Results of Aged Hair Shaft Samples

Hair shafts from 3 individuals were stored at room temperature for 6 months, 1 year, and 2 years, respectively, and detected using the system established in Example 2. As shown in Table 8, the detection results for hair shafts stored for 6 months showed little difference from those of fresh hair shafts. However, for hair shafts stored for 1-2 years, the number of detected amplicons decreased.

TABLE 8
Detection results of aged hair shaft samples

| | | Number of | |
|---|---|---|---|
| Storage time | Samples | cSNPs/MHs | Mean ± standard deviation |
| Fresh | S1 | 200 | 196 ± 5.13 |
| | S2 | 197 | |
| | S3 | 190 | |
| 6 months | S1 | 196 | 201 ± 5.13 |
| | S2 | 203 | |
| | S3 | 206 | |
| 1 year | SI | 159 | 172 ± 12.34 |
| | S2 | 176 | |
| | S3 | 183 | |
| 2 years | S1 | 189 | 186 ± 18.15 |
| | S2 | 203 | |
| | S3 | 167 | |

From the above examples, it can be concluded that the detection kit described in the present disclosure has significant application potential. It can be used for testing hair shaft samples of various conditions, and the genotyping results of co-detected sites are consistent across hair shafts from different body parts, different colors, or different storage durations. This further demonstrates the high accuracy of the detection kit described in the present disclosure for individual identification using hair shaft samples.

Although the above examples have described the present disclosure in detail, they are only a part of, not all of, the examples of the present disclosure. Other examples may also be obtained by persons based on the examples without creative efforts, and all of these examples shall fall within the protection scope of the present disclosure.

1. A method for individual identification, comprising detecting human gene-derived coding single nucleotide polymorphism (cSNP) sites; wherein the human gene-derived cSNP sites comprise 404 cSNPs shown in a following table:

| cSNP | cSNP | cSNP | cSNP | cSNP | cSNP | cSNP | cSNP |
|---|---|---|---|---|---|---|---|
| rs11464 | rs9320182 | rs2257286 | rs931749 | rs611251 | rs3845 | rs2131730 | rs583678 |
| rs1063128 | rs9320183 | rs2248473 | rs73041302 | rs557806 | rs1046373 | rs2131731 | rs372790 |
| rs3820553 | rs9486408 | rs2852468 | rs61160177 | rs610308 | rs142478896 | rs1666573 | rs432498 |
| rs6696123 | rs9486409 | rs2857670 | rs77862444 | rs11541192 | rs143258620 | rs2934418 | rs3741748 |
| rs2273584 | rs2239710 | rs2857671 | rs8106900 | rs556052 | rs145108464 | rs3742966 | rs77813527 |
| rs641615 | rs2071599 | rs2852456 | rs6508786 | rs500079 | rs148576335 | rs937248 | rs1797683 |
| rs641621 | rs1051602 | rs2857672 | rs4386247 | rs524 | rs17152448 | rs3742963 | rs8648 |
| rs1132948 | rs6436712 | rs7968466 | rs118185142 | rs527 | rs3740535 | rs3742964 | rs11755593 |
| rs4761924 | rs112558913 | rs1059837 | rs12609247 | rs7317471 | rs1864253 | rs7166305 | rs2296744 |
| rs11549949 | rs113140754 | rs15667 | rs1142825 | rs558614 | rs150285041 | rs17820431 | rs10947436 |
| rs11549950 | rs113823535 | rs2302613 | rs4242797 | rs2770928 | rs6435927 | rs17820437 | rs2296743 |
| rs6603 | rs486903 | rs2302614 | rs4589188 | rs2304704 | rs1071862 | rs11856 | rs367875497 |
| rs3818831 | rs12896 | rs1049500 | rs4589189 | rs17337252 | rs76239019 | rs8784 | rs9982775 |
| rs2832753 | rs1883 | rs201017624 | rs1131482 | rs2305427 | rs10186663 | rs12439292 | rs9982675 |
| rs2832752 | rs8020483 | rs1876363 | rs4947 | rs1129660 | rs1193 | rs1989365 | rs9982755 |
| rs10437608 | rs7143775 | rs1049205 | rs1059623 | rs7327548 | rs13297 | rs2010201 | rs9982910 |
| rs1554998431 | rs12588499 | rs2075639 | rs10792769 | rs77685055 | rs1049457 | rs12452295 | rs13046903 |
| rs3824886 | rs10289 | rs1049206 | rs2663 | rs1062087 | rs1049458 | rs8072775 | rs7275281 |
| rs3781679 | rs4246 | rs1049207 | rs2664 | rs2297208 | rs11547281 | rs7786826 | , rs7275298 |
| rs16906210 | rs1050784 | rs1049208 | rs2665 | rs557337 | rs3738126 | rs7800753 | rs7276859 |
| rs11205896 | rs2305556 | rs2037912 | rs477859 | rs4881274 | rs818571 | rs2178428 | rs12481809 |
| rs1770791 | rs1047448 | rs1049212 | rs483876 | rs10904083 | rs3750823 | rs3735576 | rs12483730 |
| rs8375 | rs1047449 | rs7214 | rs624313 | rs2288681 | rs10887673 | rs2108192 | rs2838622 |
| rs6503578 | rs3744786 | rs1128416 | rs4925229 | rs3740304 | rs74506939 | rs10269841 | rs7283084 |
| rs9635728 | rs2071561 | rs11687800 | rs11204472 | rs2640738 | rs3826543 | rs3801904 | rs9984476 |
| rs6503577 | rs72830046 | rs7475 | rs8124907 | rs2640785 | rs112542604 | rs2007922 | rs2838621 |
| rs10788862 | rs12948056 | rs3190 | rs2427289 | rs2846412 | rs141856614 | rs3807890 | rs6558394 |
| rs4890107 | rs11078993 | rs2241726 | rs6062223 | rs10749920 | rs3815561 | rs11520636 | rs10866915 |
| rs78283750 | rs2857257 | rs10747559 | rs13038331 | rs2640779 | rs3740497 | rs923935 | rs4965046 |
| rs9863910 | rs2071563 | rs3782357 | rs13042941 | rs3741060 | rs807048 | rs923936 | rs112100339 |
| rs576 | rs2604954 | rs3741622 | rs13044266 | rs4643041 | rs3829598 | rs76443569 | rs12101554 |
| rs704246 | rs2604955 | rs11168830 | rs6142734 | rs12233086 | rs9890989 | rs936524 | rs2130882 |
| rs4946758 | rs2604953 | rs7908387 | rs2151513 | rs7558074 | rs9897046 | rs12983010 | rs3803385 |
| rs1340623 | rs71373411 | rs1681 | rs3810548 | rs11896520 | rs3813048 | rs73038948 | rs3169159 |
| rs4945755 | rs743686 | rs2076190 | rs7262393 | rs1376443 | rs3813050 | rs4801861 | rs3169160 |
| rs4946759 | rs7216017 | rs2231301 | rs2427285 | rs2290257 | rs3813047 | rs1060186 | rs9652498 |
| rs11152999 | rs12451652 | rs1950252 | rs2427284 | rs9308819 | rs375280428 | rs201833096 | rs1043372 |
| rs1159148 | rs2071601 | rs10149339 | rs11698080 | rs2290258 | rs147906218 | rs2885799 | rs1062702 |
| rs3747787 | rs2239711 | rs12937519 | rs2427283 | rs77272400 | rs76327324 | rs411107 | rs1043374 |
| rs2297970 | rs79897879 | rs16966703 | rs3737137 | rs1129235 | rs80168553 | rs419374 | rs1043382 |
| rs3747790 | rs2071588 | rs2076304 | rs6143021 | rs8121 | rs72625995 | rs556681459 | rs201732843 |
| rs11537704 | rs3187034 | rs2064217 | rs2297587 | rs14039 | rs77163010 | rs11608915 | rs233309 |
| rs1057429 | rs7300801 | rs2076299 | rs6089732 | rs3173082 | rs7212517 | rs382367 | rs13051603 |
| rs1135384 | rs4761784 | rs6929069 | rs944895 | rs3777668 | rs72625994 | rs3894847 | rs58757665 |
| rs6922741 | rs4761856 | rs2076300 | rs4755571 | rs7745062 | rs6503593 | rs412533 | rs9980630 |
| rs9486407 | rs3657 | rs2744380 | rs10600 | rs7745259 | rs8067655 | rs410562 | rs233303 |
| rs13319 | rs2857663 | rs12250 | rs2074478 | rs3777667 | rs7182648 | rs570463693 | rs78731676 |
| rs1057433 | rs3741715 | rs3745951 | rs1265099 | rs8476 | rs1280396 | rs1053796 | rs3804008 |
| rs8013 | rs2857667 | rs34989303 | rs3132558 | rs17152433 | rs1620402 | rs2567 | rs3804007 |
| rs11758586 | rs2852464 | rs3745952 | rs626974 | rs3906796 | rs16977594 | rs2568 | rs2236427 |
| rs6933677 | rs2857669 | rs931750 | rs564196 | — | — | — | —; |

wherein the reagent comprises a primer set for amplifying the 404 cSNPs;
wherein the primer set comprises 326 primer pairs, and the 326 primer pairs have nucleotide sequences set forth in SEQ ID NO: 1 to SEQ ID NO: 652;
wherein the detecting human gene-derived coding single nucleotide polymorphism (cSNP) sites comprises:
extracting RNA from a hair shaft sample to be tested, and subjecting the RNA from the hair shaft sample to be tested to reverse transcription, thereby obtaining a hair shaft sample-derived CDNA:
subjecting the hair shaft sample-derived cDNA to multiplex PCR amplification using the primer pairs have nucleotide sequences set forth in SEQ ID NO: 1 to SEQ ID NO: 652 to obtain a hair shaft sample-derived multiplex PCR amplification product;
subjecting the hair shaft sample-derived multiplex PCR amplification product to library construction, thereby obtaining a hair shaft sample-derived sequencing library:
subjecting the hair shaft sample-derived sequencing library to high-throughput sequencing, thereby obtaining a high-throughput sequencing result; and
subjecting the high-throughput sequencing result to genotyping, thereby obtaining a cSNP genotyping result and/or a microhaplotype (MH) genotyping result.

2. (canceled)

3. (canceled)

4. (canceled)

5. The method according to claim 1, wherein a “reads” count threshold is set to 30 for amplicons during the genotyping.

6. A primer set for individual identification of a hair shaft, comprising 326 primer pairs, wherein the 326 primer pairs have nucleotide sequences set forth in SEQ ID NO: 1 to SEQ ID NO: 652.

7. A kit for individual identification of a hair shaft, comprising the primer set according to claim 6.

8. The kit according to claim 7, further comprising an RNA extraction-related reagent and/or an RNA reverse transcription-related reagent.

9. The kit according to claim 8, further comprising a high-throughput library construction-related reagent and/or a high-throughput sequencing-related reagent.
